(19) United States
(12) Patent Application Publication
(10) Pub. No.: US 2025/0243473 A1
(43) Pub. Date: Jul. 31, 2025

(54) FUSION OF SITE-SPECIFIC RECOMBINASES FOR EFFICIENT AND SPECIFIC GENOME EDITING

(71) Applicant: TECHNISCHE UNIVERSITÄT DRESDEN, Dresden (DE)

(72) Inventors: Frank BUCHHOLZ, Dresden (DE); Felix LANSING, Dresden (DE); Janet KARPINSKI, Dresden (DE); Teresa ROJO ROMANOS, Dresden (DE); Jan SONNTAG, Dresden (DE); Liliya MUKHAMET-ZYANOVA, Dresden (DE)

(21) Appl. No.: 17/780,136

(22) PCT Filed: Dec. 3, 2020
(86) PCT No.: PCT/EP2020/084489

§ 371 Date: May 26, 2022

(30) Foreign Application Priority Data
Dec. 6, 2019 (EP) 19214152.1

(51) Int. Cl. C12N 9/22

(52) U.S. Cl. CPC C12N 9/22; C07K 2319/00

(57) ABSTRACT

The invention relates generally to the field of genome editing and provides DNA recombinases, which efficiently and specifically recombine genomic target sequences via the fusion of recombinase monomers. More specifically, the invention provides a method to generate a fusion protein for efficient and specific genome editing, comprising a complex of recombinases comprising at least a first recombinase enzyme, a second recombinase enzyme and at least one linker, wherein said first recombinase enzyme and said second recombinase enzyme specifically recognize a first half-site and a second half-site of an upstream target site and/or a downstream target site of a recombinase; wherein said first recombinase enzyme and said second recombinase enzyme are interconnected via a linker; and wherein said linker comprises or consists of an oligopeptide. The invention further relates to fusion proteins generated with this method. The invention also discloses designer-recombinases, which catalyze the inversion of a DNA sequence present in the int1h regions on the human X chromosome. The invention further relates to nucleic acid molecules encoding said DNA recombinases and fusion proteins, as well as to the use of said fusion proteins, DNA recombinases and nucleic acid molecules in a pharmaceutical composition.

### RELATED APPLICATION DATA

This application is a 35 U.S.C. § 371 filing of International Patent Application No. PCT/EP2020/084489, filed Dec. 3, 2020, which claims priority to European Patent Application No. 19214152.1, filed Dec. 6, 2019, the contents of which are incorporated herein by reference in their entirety.

### SEQUENCE LISTING

The instant application contains a Sequence Listing which has been submitted electronically in ASCII format and is hereby incorporated by reference in its entirety. Said ASCII file, created on Oct. 23, 2023, is named 728870_TUD9-002US_ST25.txt and is 110,677 bytes in size.

### FIELD OF THE INVENTION

The invention relates generally to the field of genome editing and provides DNA recombinases, which efficiently and specifically recombine genomic target sequences via the fusion of recombinase monomers. More specifically, the invention provides a method to generate a fusion protein for efficient and specific genome editing, comprising a complex of recombinases comprising at least a first recombinase enzyme, a second recombinase enzyme and at least one linker, wherein said first recombinase enzyme and said second recombinase enzyme specifically recognize a first half-site and a second half-site of an upstream target site and/or a downstream target site of a recombinase; wherein said first recombinase enzyme and said second recombinase enzyme are interconnected via a linker; and wherein said linker comprises or consists of an oligopeptide. The invention further relates to fusion proteins generated with this method. The invention also discloses designer-recombinases, which catalyze the inversion of a DNA sequence present in the int1h regions on the human X chromosome. The invention further relates to nucleic acid molecules encoding said DNA recombinases and fusion proteins, as well as to the use of said fusion proteins, DNA recombinases and nucleic acid molecules in a pharmaceutical composition.

### BACKGROUND OF THE INVENTION

Genome engineering is becoming an increasingly important technology in biomedical research. The main approach in the field of gene editing nowadays is the nuclease-mediated introduction of double-strand breaks (DSB) at the locus of interest that are subsequently corrected by the cellular repair pathways. There are four types of programmable nucleases that can be divided in two groups based on their mode of target DNA sequence recognition. Meganucleases, zinc finger nucleases (ZFNs) and transcription activator-like nucleases (TALENs) guide the nuclease to the specific locus using protein-DNA interactions, while clustered, regularly interspaced, short-palindromic repeat-associated (CRISPR) endonucleases direct it using RNA-DNA interactions (Caroll, 2014; Wang et al., 2017). Programmable nucleases are candidates for therapeutic application, and several of them are already in clinical trials (Tebas et al., 2014; Qasim et al., 2015; Cyranoski, 2016).

Yet, one of the main challenges of programmable nucleases is the risk of unpredictable sequence rearrangements. The introduced DSBs are repaired by the cells primarily using non-homologous end-joining (NHEJ) or homology-directed repair (HDR). The repair by HDR is precise and maintains genomic stability, as the sequence is copied from the second allele or a donor sequence that matches the target. However, HDR is mainly active during DNA replication, and in most cells NHEJ events outnumber HDR. NHEJ is an error-prone repair mechanism, which leads to insertions and deletions (indels) in the repaired DNA fragment. This may result in adverse events due to alteration of gene sequence. (Caroll, 2014; Cox et al., 2015; Kosicki et al., 2018)

Alternative tools that are widely used for genome engineering include site-specific recombinases (SSRs) from the tyrosine recombinase family. Tyrosine SSRs have considerable advantages over programmable nucleases, as they are not dependent on the cellular DNA repair pathways because they perform the full recombination reaction without any accessory factors (Meinke et al., 2016). This leads to highly specific, predictable and precise genome editing events, which makes them attractive for therapeutic applications.

One of the most commonly used SSRs is Cre—a tyrosine recombinase from the E. coli bacteriophage P1 that recognizes 34 bp symmetric loxP sites, consisting of two 13 bp palindromic sequences flanking an 8 bp spacer (FIG. 1).

The recombination reaction carried out by SSRs is a multistep process. First, four single recombinase molecules bind to each palindromic half-site of loxP, forming an active recombination synapsis (FIG. 2A). At this stage, one of the recombinases of each of the dimers is activated, and performs the cleavage of the DNA. The cleavage occurs due to nucleophilic attack of tyrosine at position 324 of Cre to the scissile phosphate in the spacer of the loxP site. This leads to the formation of a 3′-phospho-tyrosine intermediate and release of a 5′-hydroxyl group. This step is followed by the strand exchange caused by the attack of the other 3′-phospho-tyrosine linkage by the released 5′-hydroxyl group, and the formation of a Holiday Junction intermediate. Next, the Holiday Junction is isomerized, and the structure of the molecules change, in that the active pair of Cre molecules become inactive and the previously inactive Cre molecules become activated. The same process of cleavage and exchange is then repeated with the second set of strands (Meinke et al., 2016).

The result of recombination depends on the orientation of the spacer. If the spacer sequences of two loxP sites are oriented in the same direction, the recombination leads to excision or integration of the DNA fragment (FIG. 2B). If the spacers are in the inverted orientation, the recombination results in inversion of the flanked DNA sequence (Meinke et al., 2016).

In order to use the Cre recombinase system, the loxP sites are typically introduced via genome engineering at the desired genomic locus. While this approach has been successfully used for conditional mutagenesis in animal models for investigation of gene function and modeling of genetic events underlying human diseases (Albanese et al., 2002; Justice et al., 2011), it greatly limits its application in humans. To overcome this restriction, techniques to alter the recombinase enzyme to make it recombine an artificial DNA sequence have been developed, for example, to excise the HIV provirus from infected cells (Buchholz and Stewart, 2001; Santoro and Schultz, 2002; Sarkar et al., 2007; Karpinski et al., 2016). The palindromic nature of the target site considerably confines the number of possible target sites in genomes that can be recombined. Nevertheless, previous work has shown that single SSRs can be generated that specifically recombine asymmetric target sites (Sarkar et al., 2007; Karpinski et al., 2016). Another possibility to recombine asymmetric target sites is to build heterospecific recombinase systems, where two different recombinase monomers bind to the respective half-site and both enzymes cooperatively recombine the final asymmetric target site (FIG. 3) (Saraf-Levy et al., 2006). That such systems can indeed be developed for advanced genome editing in human cells has recently been demonstrated (Lansing et al., 2020). Therefore, these systems could significantly expand the utility of SSR systems.

However, the single recombinases of the heterodimer could also form homodimers with activity on symmetric sites. Therefore, using two recombinases instead of one bears the potential risk of having more off-target events since each recombinase of a heterodimer has its own symmetric off-target sites in addition to the putative asymmetric heterodimer off-targets. In order to use a heterodimer for therapeutic application, high efficacy and specificity of the system should be ensured. One of the options would be modification of the heterodimer that prevents the individual recombinases from performing recombination as homodimers. In this scenario, the activity on the symmetric sites could potentially be eliminated or restricted. Therefore, it is of high interest to generate systems that make recombinase heterodimers obligate.

Consequently, several approaches have been developed to increase Cre specificity, including the generation of obligate heterodimers with “hydrophobic size switch” (Gelato et al., 2008), an obligate heterotetrameric complex with selective destabilization (Zhang et al., 2015) or destabilizing binding cooperativity (Eroshenko and Church, 2013). However, these alterations all lead to marked reduction in recombination activity that are likely to be incompatible with efficient genome alterations for therapeutic applications.

One of the human diseases that can possibly be corrected by SSRs is Hemophilia A (HA). HA is an X-linked monogenetic disease caused by defects in the F8 gene that encodes the blood clotting factor VIII (FVIII). The F8 gene is located on the long arm of the X chromosome (Xq28). The gene spans 186 kb, and consists of 27 exons.

FVIII is mainly synthesized in Liver Sinusoidal Endothelial Cells (LSECs) and other human endothelial cells (Shahani et al., 2014; Turner and Moake, 2015). FVIII is an important component of the blood coagulation cascade. FVIII circulates in the blood bound to von Willebrand factor, and dissociates from it after the activation by thrombin. Activated FVIII (FVIIIa) binds FIXa and this complex subsequently activates FX, leading to a chain of reactions forming a blood clot (Dahlback, 2000).

HA affects 1 in about 5,000 males (Graw et al., 2005). Clinical severity of HA depends on the residual activity level of the factor VIII in the blood and is classified into three groups: mild (5-40% of normal level), moderate (1-5%), severe (<1%) (White et al., 2001). Patients with mild HA experience bleeding episodes only in cases of significant traumas or surgeries. Moderately affected individuals have spontaneous bleedings after trivial trauma. Severe HA is characterized by frequent spontaneous bleedings into internal organs, muscles and joints. Repeated hemarthroses often lead to the disabling condition called hemophilic arthropathy. This complication is characterized by chronic pain, joint impairment and dramatic decrease of patients' lives quality (Pandey and Mittal, 2001; Fischer et al., 2005; Melchiorre et al., 2017).

More than half of the HA patients have the severe form of the disease, and in most of the cases it is caused by a genomic inversion due to intrachromosomal recombination with homologous regions outside of the F8 gene (Graw et al., 2005).

The second most common genetic alteration in patients with severe HA (˜1-5%) is the inversion of exon 1. It happens due to homologous recombination between two 1 kb sequences (int1h), in intron 1 and in the region positioned telomeric to the F8 gene (FIG. 4). The sequences are located in opposite orientation and in a distance of approximately 140 kb. Homologous recombination leads to the inversion and translocation of exon 1, thus, resulting in the disruption and dysfunction of the F8 gene (Castaldo et al., 2007; Tizzano et al., 2003).

The standard treatment for severe HA is a substitution therapy—administration of plasma-derived or recombinant concentrate of blood coagulation factor VIII. However, due to their short half-lives (8-12 hours), very frequent administration (two to three times weekly or even every other day) is necessary. This procedure has a great impact on patients' quality of life (Peyvandi et al., 2016). Several modifications on the protein have been developed to increase the half-lives of the recombinant FVIII, such as PEGylation and fusion with the fragment of an IgG, that allowed to obtain the therapy with comparable efficacy but with the half-life 1.5-fold longer than that of the standard FVIII infusion (Mahlangu et al., 2014; Konkle et al., 2015; Peyvandi et al., 2016). The extension of the half-lives leads to a small reduction of administration frequency resulting from two injections per week to one injection every 5 days (Carcao, 2014).

In addition to the frequent intravenous injections, a great challenge of the current treatment is that some of the patients (around 30%) develop inhibitory antibodies against the administrated FVIII, thereby compromising the treatment (Gouw et al., 2013). Moreover, the economic burden of HA should be taken into consideration. The average annual direct cost of the standard treatment is around EUR 200,000 in Europe, while in Germany it is more than EUR 300,000 per patient (O'Hara et al., 2017). Thus, only the prophylaxis of HA patients without complications is extraordinary expensive. Therefore, it is important to develop other treatment options or even a curative therapy.

Alternative therapies that inhibit the anticoagulant pathways in order to achieve hemostasis were developed and some of them, such as siRNA degrading antithrombin III mRNA and monoclonal antibodies inhibiting Tissue Factor Pathway Inhibitor or Protein C, are currently in stage I-III of clinical trials. The advantage of these approaches is the possibility of being used by all the patients, with and without FVIII inhibitors, as well as the lower treatment frequency (once per week) and subcutaneous mode of administration. However, clinical studies show that the dose of the treatment has to be carefully adjusted, since overdosing increases the risk of thrombotic effects. (Peters and Harris, 2018)

Another approach is to use the FVIIIa mimetic bispecific antibody Emicizumab. This antibody bridges FIXa to FX. Therefore, it basically mimics the function of FVIII. However, this approach lacks the regulation by the thrombin cleavage. Thus, it cannot be inactivated and relies on other steps of the coagulation cascade, also having a high risk of thrombotic effects (Lenting et al., 2017).

The monogenic nature of the disease makes HA an attractive target for gene therapy. Four vectors based on the Adeno-Associated Virus (AAV) serotype that transduce hepatocytes are currently in clinical trials. This treatment has shown improvements for some patients. However, the safety of the approach still has to be investigated, since one of the main side effects is elevation of liver enzymes (Pasi et al., 2017; George et al., 2017; Peters and Harris, 2018). Furthermore, the non-integrative nature of AAV limits the application of the therapy in children, due to dilution and loss of the vector expression as a result of cell proliferation during organ growth (Vandamme et al., 2017).

The therapies described above compensate the defective FVIII gene function, but do not correct the causing mutation. Having the gene inverted back to the normal orientation would allow for stable expression of FVIII under physiological conditions. Importantly, increase of the FVIII levels to 1-5% of normal would significantly improve lives of patients with severe HA, because it would greatly lower the risk of internal spontaneous bleedings and the administration of the FVIII would be needed only in case of traumas and surgeries.

The group of Jin-Soo Kim has used programmable nucleases to correct two inversions causing severe HA. They succeeded in inverting exon 1 for 0.2-0.4% using ZFNs in HEK 293 cells (Lee et al., 2012) and for 1.4% using TALENs (Park et al., 2014) in human iPSCs. The most efficient correction (reversion) of 6.7% for exon 1 and 3.7% for the exons from 1 to 22 in the F8 gene in HA patient-derived iPSCs was achieved using CRISPR-Cas9 technology (Park et al., 2015). While impressive, the obtained results with nucleases have limited therapeutic utility. Programmable nucleases perform the correction by the introduction of two double strand breaks in the homologous regions, resulting in either inversion or deletion of the flanked DNA fragment made by the cellular repair pathways. In addition, indels at the cutting sites are also likely to occur. Therefore, the inversion performed by ZFN, TALEN or CRISPR-Cas9 is more a stochastic than a controlled event.

To solve this shortcoming, SSRs could be used to correct genetic inversions causing diseases. SSRs are highly specific, and can carry out inversions of DNA sequences in a genomic context, without dependence on any accessory factors (Yu and Bradley, 2002) when target sites for the recombinase are present in the appropriate orientation. The objective therefore was to develop an SSR system that can correct the int1h inversion in human cells at high efficacy and specificity.

### SUMMARY OF THE INVENTION

The problem of the present invention is therefore to provide DNA recombinases, which efficiently and specifically recombine genomic target sequences. In particular, the problem has to be solved that each individual recombinase monomer has to find and recognize its binding site efficiently and specifically.

The problem of the invention is solved by the provision of method of generating a fusion protein or DNA recombinase for efficient and specific genome editing, in particular for recombination, most preferably for inversion of DNA sequences on genomic level in a cell, comprising a complex of recombinases comprising at least a first recombinase enzyme, a second recombinase enzyme and at least one linker, wherein said first recombinase enzyme and said second recombinase enzyme specifically recognize a first half-site and a second half-site of an upstream target site and/or a downstream target site of a recombinase; wherein said first recombinase enzyme and said second recombinase enzyme are interconnected via a linker; and wherein said linker comprises or consists of an oligopeptide.

The invention relates further to the non-fused heterodimer DNA recombinases without the linker peptide, especially for use in the recombination, most preferably the inversion of DNA sequences on genomic level in a cell.

With this invention it is shown, e.g. in FIGS. 19, 20 and 23, that recombinase monomers can be tethered together into a single polypeptide without losing the ability to recombine the respective target site. This is unexpected, given the known molecular details of the recombination reaction (stepwise cleavage, Holiday structure formation and isomerization). The current dogma was that the 4 recombinase molecules necessary to carry out the recombination reaction have to be flexible monomers to be able to function as an enzymatic complex. It is shown herein that fusion of SSRs via specific linkers can be applied to designer-recombinases, evolved to recombine a sequence present in inverted repeats, causally involved in a genetic alteration of the FVIII gene causing HA in humans. Unexpectedly, fusion of two heterospecific recombinases with specific linkers prevent the molecule from recombining the symmetric target sites, making the system obligate. Furthermore, the fusion protein has high activity and shows greatly improved specificity in comparison to the non-linked enzymes. Directed molecular evolution lead to an improved linker sequence with desirable features.

The invention also discloses a target sequence in the int1h sequences (loxF8) implicated in the FVIII inversion, needed for the generation of designer-recombinases.

The problem of the invention is in particular solved by providing a fusion protein for efficient and specific genome editing, comprising a complex of recombinases comprising at least a first recombinase enzyme, a second recombinase enzyme and at least one linker, wherein said first recombinase enzyme and said second recombinase enzyme specifically recognize a first half-site and a second half-site of an upstream target site and/or a downstream target site of a recombinase; wherein said first recombinase enzyme and said second recombinase enzyme are interconnected via a linker; and wherein said linker comprises or consists of an oligopeptide comprising 4 to 50 amino acids.

The invention further relates to DNA recombinases, which specifically recognize upstream and downstream target sequences of the loxF8 recombinase target sites, and which catalyze the inversion of a gene sequence between these upstream and downstream target sequences of the loxF8 recombinase target sites.

The invention relates further to nucleic acid molecules encoding a fusion protein or a DNA recombinase according to the invention.

In a further embodiment, the invention provides a mammalian, insect, plant or bacterial host cell comprising said nucleic acid molecule encoding a fusion protein or DNA recombinase according to the invention.

A fusion protein or a DNA recombinase or a nucleic acid molecule according to the invention can be used as a medicament and can therefore be comprised in a pharmaceutical composition, optionally in combination with one or more therapeutically acceptable diluents or carriers.

The fusion protein or DNA recombinase or the pharmaceutical composition according to the invention are suitable for the treatment of a disease that can be cured by genomic editing, in particular the treatment of hemophilia A.

In a further embodiment, a method for determining recombination on genomic level in a host cell culture or patient, comprising a fusion protein or DNA recombinase for efficient and specific genome editing according to the invention, is provided.

### BRIEF DESCRIPTION OF THE DRAWINGS

FIG. 1 is a scheme of Cre molecules binding its target loxP site. The loxP site consists of two inverted repeats (13 bp) flanking a non-palindromic spacer (8 bp). Two Cre molecules bind to the loxP site, each to one of the half-sites.

FIG. 2 represents a scheme of the tyrosine SSR recombination reaction. (A) Schematic of the stepwise recombination mechanism. Four recombinase molecules bind two DNA substrates, forming a tetrameric complex (1). Recombinases in the “cleaving competent” conformation are shown in light grey, the once in the “noncleaving” conformation are shown in dark grey. The nucleophilic tyrosine is indicated as a Y in a light grey circle and shown only for the active monomers. The activated nucleophilic tyrosine attacks the scissile phosphate (indicated as a P), forms a 3′-phosphotyrosine linkage and leads to release of free 5′OH (2). The released 5′OH attacks the neighboring phosphotyrosine, forming a Holiday Junction Intermediate and leading to strand exchange (3). The complex is isomerized and the active monomers change the conformation to inactive and vice versa (4). The cleavage and strand exchange steps are repeated (5,6). (B) Possible outcomes of the recombination reaction. Recombination reactions can lead to excision/integration or inversion of the DNA fragment, depending on the orientation of target sites (spacer sequence). The target sites are indicated as black triangles, and their directionality indicates the orientation of the target sites. (Meinke et al., 2016).

FIG. 3: shows a schematic model of recombination by a heterotetrameric Cre complex. Black-shaded ellipses represent wild type Cre monomers, grey-shaped ellipses represent mutated Cre monomers, each bound to different half-sites of loxP sites (black-shaded strands represent the original loxP site and the grey-shaded strand represent the half-site recognized by the mutated Cre) (Saraf-Levy et al., 2006).

FIG. 4: shows a schematic of a F8 gene inversion causing severe forms of HA. F8 gene or its part is depicted, exons are shown in as arrows, introns are shown in white boxes, the line represents DNA outside the F8 gene. A. Inversion of exon 1 is caused by recombination between the homologous regions in intron 1 (int1h1) and on the telomeric side outside of the F8 gene (int1h2). The crossover is represented by splitting the int1h1 and int1h2 boxes. The inverted situation is shown on the bottom with the two chosen target sequences (loxF8, SEQ ID NO: 17) bound by designer-recombinases (S1-specific, black and S2-specific, white). Note the inverted orientation of the spacer sequence.

FIG. 5: shows a scheme of the F8 heterodimer (A) and the single monomer (B) recognizing the loxF8 target site. The single monomer recombinase (H7, SEQ ID NO: 66, grey) recognizes both half-sites, whereas the S1-specific recombinase recognizes the left half-site 1 (SEQ ID NO: 32, black) and the S2-specific recombinase recognizes the right half-site 2 (SEQ ID NO: 33, white).

FIG. 6: shows the recombination activity comparison of the single F8-recombinase (H7, SEQ ID NO: 66) with the dual F8 recombinase (D7, SEQ ID NOs: 32 and 33) in E. coli. Digested plasmid DNA isolated from cells harboring the pEVO-loxF8 plasmid expressing the indicated recombinases at indicated concentrations of Arabinose (Ara) are shown. The line with two arrows marks the non-recombined bands. The line with one arrow marks the recombined bands. Note the favorable recombination activity of the D7 heterodimer recombinase at all tested conditions. M, marker.

FIG. 7: shows the recombination activity comparison of the single F8-recombinase (H7, SEQ ID NO: 66) with the dual F8 recombinase (D7, SEQ ID NO: 32 and 33) for transient transfections in mammalian cells. (A) Illustration of the employed plasmids. (B) Results determined by FACS analyses. The overall recombination efficiencies are indicated below the bars in % where the left column shows D7 heterodimer and the right column H7 monomer.

FIG. 8: shows that the F8 recombinases invert the genomic F8 locus in human tissue culture cells. A PCR assay detecting the genomic inversion in genomic DNA isolated from HEK293 cells transfected with indicated recombinase expression constructs is shown. The arrow marks the specific inversion band. Note the prominent band obtained for cells transfected with the D7 heterodimer construct, indicating efficient inversion. In contrast, the H7 monomer transfected cells only display a weak band, signifying that the inversion was less efficient. C, control (non-transfected cells); M, marker.

FIG. 9: shows the off-target analyses of F8 recombinases. Digested plasmid DNA isolated from cells harboring the pEVO-loxHS1-9 plasmids expressing the indicated recombinases (H7 monomer, D7 heterodimer). The lines with two arrows mark the non-recombined bands. The lines with one arrow mark the recombined bands. Note that the H7 monomer shows the recombination specific bands in several lanes. M, marker.

FIG. 10: shows the symmetric target sites deduced from the two loxF8 half-sites. (A) The sequence of the symmetric Sym1 site is shown bound by two monomers of an S1-specific recombinase (black). (B) The sequence of the symmetric Sym2 site is shown bound by two monomers of an S2-specific recombinase (white).

FIG. 11: shows possible binding scenarios of fused heterospecific recombinases to symmetric and asymmetric loxF8 target sites.

FIG. 12: shows the recombination efficacy of the recombinase monomers, non-linked heterodimer and S2-(G₂S)₈-S1 on the loxF8, Sym1 and Sym2 target sites. (A) Recombination activity of the S1-specific (S1) and S2-specific (S2) recombinase monomers. Note the absence on recombination of the individual enzymes on the asymmetric loxF8 site. In contrast, the S1 monomer shows activity only on the symmetric Sym1 target site, while S2 shows activity only on the symmetric Sym2 target site. (B) Recombination activity of the non-fused recombinase heterodimer. Note that all target sites are recombined when both recombinases are expressed together in the same cell. (C) Recombination activity of the fused recombinase heterodimer S2-(G₂S)₈-S1. Note that only the asymmetric loxF8 site is recombined, while both, Sym1 and Sym2, are not recombined by the fusion recombinase. 200 μg/ml L-arabinose was used for induction for all experiments. Recombination of the plasmid DNA was assayed by restriction enzyme digestion (SacI-HF+SbfI-HF), resulting in a smaller fragment for recombined (one triangle) and a bigger fragment for unrecombined plasmids (two triangles).

FIG. 13: shows a scheme of the linker libraries design. Three oligonucleotides to be used for PCR. Each of them contained two adapters from each side for primer annealing, XhoI and BsrGI-HF restriction sites, and linker sequence. Part or the whole amino acid sequence of the (G₂S)₈ sequence was changed to degenerate codons RVM. Nucleotide and amino acid sequences are indicated where appropriate.

FIG. 14: shows the linker selection on the asymmetric loxF8 site. (A) Initial recombination activity of the heterodimer fused with three different linker libraries (lib1-3) on the loxF8 site. 200 μg/ml L-arabinose was used for induction. Recombination of the plasmid DNA was assayed by restriction enzyme digestion (SacI-HF+SbfI-HF), resulting in a smaller fragment for recombined (one triangle) and a bigger fragment for unrecombined plasmids (two triangles). (B) Scheme of the linker selection on the loxF8 site. Digestion with NdeI and AvrII restriction enzymes takes place only on the non-recombined plasmids, therefore the PCR product with indicated primers (F and R) cannot be obtained from these plasmids. Only the linked heterodimers that have recombined the loxF8 site are amplified by PCR, allowing to select only active fusion proteins.

FIG. 15: shows the linker counter-selection on the symmetric target sites. (A) Recombination activity of the heterodimer fused with indicated linker libraries (lib1-3) on the Sym1 and Sym2 sites. 200 μg/ml L-arabinose was used for induction. Recombination of the plasmid DNA was assayed by restriction enzyme digestion (SacI-HF+SbfI-HF), resulting in a smaller fragment for recombined (one triangle) and a bigger fragment for unrecombined plasmids (two triangles). (B) Scheme of the linker counter-selection on the symmetric target sites, shown on the example of Sym1. The reverse primer (R) anneals between two symmetric sites, therefore the PCR product cannot be obtained from the recombined plasmids, as this DNA fragment is excised during the recombination. Only the linked heterodimers that have not recombined the symmetric site are amplified by PCR, that allows to select only fusion proteins that are not active on the symmetric sites.

FIG. 16: shows the final recombination activity of the evolved libraries on the loxF8, Sym1 and Sym2 sites. 1 μg/ml and 200 μg/ml L-arabinose were used for induction on the asymmetric loxF8 and symmetric Sym1 and Sym2 sites, respectively. Recombination of the plasmid DNA was assayed by restriction enzyme digestion (SacI-HF+SbfI-HF), resulting in a smaller fragment for recombined (one triangle) and a bigger fragment for unrecombined plasmids (two triangles).

FIG. 17 shows clonal analyses of fused D7 heterodimers on the loxF8, Sym1, and Sym2 sites. Recombination activity of 4 clones from each library (L1, L2, L3, L4 from S2-link lib1-S1, L5, L6, L7, L8 from S2-link lib2-S1, L9, L10, L11, L12 from S2-link lib3-S1) was analyzed on the loxF8 (A), Sym1 (B), and Sym2 (C) target sites. 10 μg/ml and 200 μg/ml L-arabinose were used for induction on the asymmetric loxF8 and symmetric Sym1 and Sym2 sites, respectively. Recombination of the plasmid DNA was assayed by restriction enzyme digestion (SacI-HF+SbfI-HF), resulting in a smaller fragment for recombined (one triangle) and a bigger fragment for unrecombined plasmids (two triangles). Clone L8 was chosen for further analyses based on its high recombination activity on loxF8 and its lack of recombination on Sym1 and Sym2.

FIG. 18: shows the comparison of the recombination efficacy of the D7 heterodimer fused with (G₂S)₈ and L8 linkers on the loxF8 target site. Recombination activity of the recombinase heterodimer fused with (G₂S)₈ or the L8 linker on the loxF8 site. L-arabinose concentrations from 0 to 250 μg/ml were used for recombinase induction as indicated. Recombination of the plasmid DNA was assayed by restriction enzyme digestion (SacI-HF+SbfI-HF), resulting in a smaller fragment for recombined (one triangle) and a bigger fragment for unrecombined plasmids (two triangles). Note the increased activity of the L8 linked recombinases.

FIG. 19: shows the recombination activity of the non-linked D7 heterodimer and the S2-linker L8-S1 recombinases on the asymmetric loxF8 target site. Recombinases were assayed in the pEVO-SDO vector. L-arabinose concentrations from 0 to 50 μg/ml were used for recombinase induction as indicated. Recombination of the plasmid DNA was assayed by restriction enzyme digestion (SacI-HF+SbfI-HF), resulting in a smaller fragment for recombined (one triangle) and a bigger fragment for unrecombined plasmids (two triangles). Note the increased activity of the L8 linked recombinases. SDO; shine dalgano optimized.

FIG. 20: shows the recombination activity of two recombinase heterodimers (D7 and A4) as non-fused and fused versions on predicted human genome off-target sites. The fusion of recombinases was performed in a S2-linker L8-S1 fashion (FIG. 20A). Recombination activity of the non-linked D7 heterodimer and the D7 heterodimer fused with the L8 linker (comprising the recombinase monomers of SEQ ID NOs. 32 and 33) on asymmetric (1LR, 2LR, 3LR, 4LR, 5LR) and symmetric (1L, 2L, 1R, 2R) off-target sites are shown. 50 μg/ml and 250 μg/ml L-arabinose were used for induction of the non-linked heterodimer and S2-linker L8-S1, respectively. Recombination of the plasmid DNA was assayed by restriction enzyme digestion (SacI-HF+SbfI-HF), resulting in a smaller fragment for recombined (one triangle) and a bigger fragment for unrecombined plasmids (two triangles). Note that the L8-fused D7 heterodimer does not show marked off-target recombination, whereas off-target recombination can be observed for the non-fused version on target sites 2LR and 2L. FIG. 20B shows the recombination activity of the non-linked A4 heterodimer and the S2-linker L8-S1 (fused A4) recombinases on predicted human genome off-target sites. Recombination activity of the non-linked A4 heterodimer and the A4 heterodimer fused with the L8 linker (comprising the recombinase monomers of SEQ ID NOs. 93 and 94) on asymmetric (1LR, 2LR, 3LR, 4LR, 5LR) and symmetric (1L, 2L, 1R, 2R) off-target sites are shown. 100 μg/ml was used for induction of the non-linked heterodimer and S2-linker L8-S1, respectively. Recombination of the plasmid DNA was assayed by restriction enzyme digestion (SacI-HF+SbfI-HF), resulting in a smaller fragment for recombined (one triangle) and a bigger fragment for unrecombined plasmids (two triangles). In contrast to the D7 clone (non-fused), the L8-fused A4 heterodimer as well the non-fused version do not show marked off-target recombination at any target site tested.

FIG. 21: shows a scheme of the recombinase expression plasmids used in human cells. (A) Illustration of the plasmids for expression of single recombinases (S1- or S2-specific). (B) Illustration of the expression plasmid for the fused D7 heterodimer (S2-linker L8-S1). For all expression plasmids EGFP expression is coupled to recombinase expression through a self-cleaving 2A peptide from porcine teschovirus-1 polyprotein (P2A). The arrows mark transcription start sites from the EF-1a promoter.

FIG. 22: shows a scheme of the mCherry gene-based reporter assay. The reporter plasmid contains stop codons between the two loxF8 target sites that prevents the expression of mCherry. Upon recombination, the stop codon is removed, resulting in constitutive expression of mCherry conferring red color to cells. The arrows mark transcription start sites from the chicken beta-actin (CAG) promoter.

FIG. 23: shows flow cytometry analyses of the reporter assay in human cells. Plots of the flow cytometry analyses of HEK 293T cells transfected with the non-fused and fused D7 heterodimer (L8) (comprising the recombinase monomers of SEQ ID NOs. 32 and 33) expressing plasmids and mCherry reporter plasmid are shown. Cells expressing the D7 heterodimers are identified by expression of the green fluorescent protein (GFP+). Cells that have recombined the reporter plasmid are identified by the expression of red fluorescent protein (mCh+).

FIG. 24: shows the inversion efficacy performed by the heterodimers in mammalian cells. The line chart represents the results of qPCR performed on genomic DNA extracted 48 hours after transfection with D7 recombinase heterodimer expression vectors. The line presents the linear regression model of the standard curve with R²=0, 9918. Relative inversion of the non-fused and fused (L8) recombinase heterodimers is shown. Note the increased inversion frequency of the fused L8 heterodimer.

FIG. 25: shows the activity of recombinases on an integrated loxF8 genomic reporter in mammalian cells.

A) Depiction of the genomic reporter construct stably integrated in the genome of HEK293T cells. Step 1: A constitutive active promoter drives the expression of the puromycin (PURO) gene flanked by the loxF8 target sites (small black triangles). The subsequent fluorescent mCherry gene is not expressed. Step 2: After successful recombination of the loxF8 target sites the puromycin gene is excised and the fluorescent mCherry gene is expressed.

B) Overview of the synthetic mRNA encoding for the recombinase monomers, the recombinase fusion protein or the transfection reporter (tagBFP). The mRNAs are used to transfect the loxF8 reporter cell line.

C) Flow cytometry analyses of the loxF8 genomic reporter cell line 48 h after delivery of recombinase mRNA (non-fused D7, fused D7, non-fused A4 and fused A4) and transfection control mRNA (tagBFP). Transfected cells are identified by the expression of the blue fluorescent protein (tagBFP+). Cells that have recombined the loxF8 genomic reporter are identified by the expression of the red fluorescent protein (mCherry+).

FIG. 26: shows the analysis of the recombination event (inversion) of the native loxF8 locus after recombinase expression in mammalian cells.

A) Graphic depiction of the Factor VIII gene and the surrounding inverted homologous regions (int1h-1 and int1h-2) in normal orientation and inverted orientation (causing Hemophilia A). The loxF8 target sites are indicated with vertical arrows at the int1h-1 and int1h-2 regions. The promoter of the Factor VIII gene is depicted in front of exon 1. Primer binding sites to detect the orientation of the 140 kb fragment between the loxF8 target sites are shown with horizontal arrows (P1, P2 and P3).

B) PCR on genomic DNA of human cells 48 h after transfection with recombinases encoding mRNA (non-fused D7, fused D7, non-fused A4, fused A4 and D11 non-fused). A PCR product generated with primer pair P2 and P3 will detect the normal orientation of the loxF8 gene inversion locus (normal orientation). A PCR product generated with primer pair P1 and P3 will detect the inverted orientation of the loxF8 gene inversion locus (inverted orientation). Only cells transfected with non-fused D7, fused D7, non-fused A4, fused A4 and D11 non-fused show the inverted orientation PCR product. As inversion control, genomic DNA from a hemophilic donor carrying the Factor VIII gene inversion was used. As wild-type (WT) control genomic DNA from a healthy donor was used. No template control is shown (water control).

FIG. 27: shows a sequence alignment of the recombinases discussed wherein:
F8_D7-L is the polypeptide of SEQ ID NO: 32;
F8_D11-L is the polypeptide of SEQ ID NO: 99;
F8_A4-L is the polypeptide of SEQ ID NO: 93;
F8_28-L is the polypeptide of SEQ ID NO: 30;
Brec is the polypeptide of SEQ ID NO: 70;
Hex D12-R is the polypeptide of SEQ ID NO: 106;
Hex D12-L is the polypeptide of SEQ ID NO: 105;
Tre is the polypeptide of SEQ ID NO: 69;
Cre is the polypeptide of SEQ ID NO: 68;
F8_H7 is the polypeptide of SEQ ID NO: 66;
F8_28-R is the polypeptide of SEQ ID NO: 31;
F8_D7-R is the polypeptide of SEQ ID NO: 33;
F8_D11-R is the polypeptide of SEQ ID NO: 100; and
F8_A4-R is the polypeptide of SEQ ID NO: 94.

Black background/white bold letters: Amino acid changes that have not been reported in published Cre-derived recombinases.

Grey: Amino acid changes that have been reported in published Cre-derived recombinases.

#: Predicted critical residues for target site recognition based on deep sequencing.

Relevant PMIDs: 17600219, 26900663, 31745551

### General Definitions

As used herein, the expressions “cell”, “cell line,” and “cell culture” are used interchangeably and all such designations include progeny. Thus, the words “transformants” and “transformed cells” include the primary subject cell and culture derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, this will be clear from the context.

The terms “polypeptide”, “peptide”, and “protein”, as used herein, are interchangeable and are defined to mean a biomolecule composed of amino acids linked by a peptide bond.

If peptide or amino acid sequences are mentioned herein, each amino acid residue is represented by a one-letter or a three-letter designation, corresponding to the trivial name of the amino acid, in accordance with the following conventional list:

| Amino Acid | One-Letter Symbol | Three-Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

The terms “a”, “an” and “the” as used herein are defined to mean “one or more” and include the plural unless the context is inappropriate.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human being sought by a researcher, veterinarian, medical doctor or other clinician, which includes alleviation of the symptoms of the disease or disorder being treated.

As used herein, the term “pharmaceutically acceptable” embraces both human and veterinary use: For example, the term “pharmaceutically acceptable” embraces a veterinarily acceptable compound or a compound acceptable in human medicine and health care.

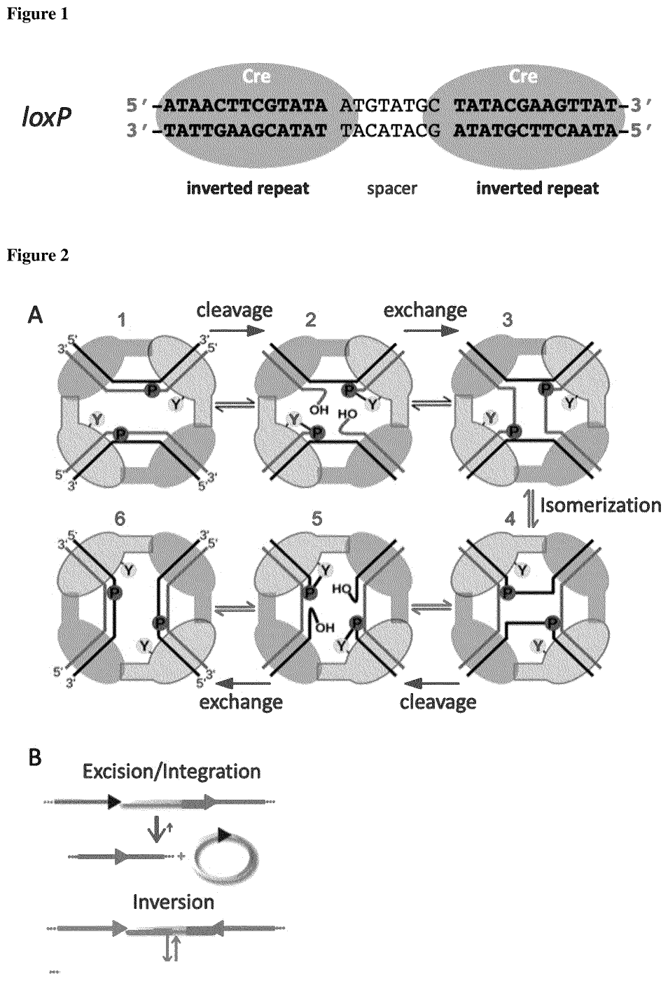

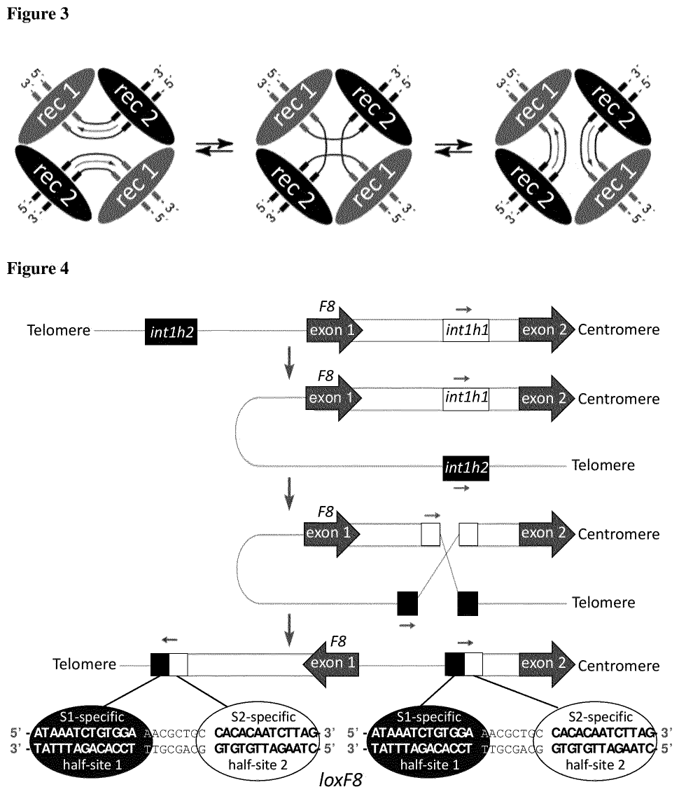

Naturally occurring DNA recombining enzymes, in particular site-specific recombinase (SSR) systems (such as tyrosine-type SSRs), generally consist of four identical monomers. In general, they recognize two identical and symmetric, palindromic target sites, which consist each of two about 13 nucleotide long half-sites separated by an asymmetric frequently 8 nucleotide long spacer (FIG. 1). Depending on the number and relative orientation of the target sites and their spacers, the DNA recombining enzyme either performs an excision, an integration, an inversion or a replacement of genetic content (FIG. 2; reviewed in Meinke et al., 2016).

As used herein, “upstream” means the 5′ target site of a recombinase in a DNA, comprising a first half-site, such as a left half-site, and a second half-site, such as a right half-site, wherein said first half-site and said second half-site are separated by a spacer sequence.

As used herein, “downstream” means the 3′ target site of a recombinase in DNA, a first half-site, such as a left half-site, and a second half-site, such as a right half-site, wherein said first half-site and said second half-site are separated by a spacer sequence.

### DETAILED DESCRIPTION OF THE INVENTION

The invention provides a method for generating a DNA recombinase for efficient and specific genome editing, in particular for recombination, most preferably for inversion of DNA sequences on genomic level in a cell, wherein said method comprises the steps of:
i. identifying a nucleic acid sequence that is a potential target site for DNA-recombining enzymes that are capable to induce a site-specific DNA recombination of a sequence of interest in a genome, wherein said potential target site comprises two asymmetric recombinase target sites;
ii. providing a nucleic acid molecule encoding a first recombinase enzyme, wherein said first recombinase enzyme has been evolved by directed evolution or rational design to specifically recognize a first half-site of a recombinase target site;
iii. providing a nucleic acid molecule encoding a second recombinase enzyme, wherein said second recombinase enzyme has been evolved by directed evolution or rational design to specifically recognize a second half-site of a recombinase target site;
iv. creating an expression vector by cloning the nucleic acid molecule encoding a first recombinase enzyme and the nucleic acid molecule encoding a second recombinase enzyme into an expression vector;
v. transfecting a cell, which comprises a DNA sequence, which is to be inverted, with the expression vector of step iv);
vi. expressing the DNA recombinase comprising a first recombinase enzyme and a second recombinase enzyme;
vii. analyzing, whether the DNA recombinase expressed in step vi. is capable of inverting the DNA sequence on a human chromosome in said cell; and
viii. selecting a DNA recombinase which is capable of inverting the DNA sequence on a human chromosome in said cell according to step vii.

It has surprisingly been found that the specificity of a DNA recombinase that has been obtained by this method, can be dramatically increased by linking the recombinase monomers with an oligopeptide, thereby producing a fusion protein.

Accordingly, in a preferred embodiment, the invention provides a method for generating a fusion protein for efficient and specific genome editing, in particular for recombination, most preferably for inversion of DNA sequences on genomic level in a cell, wherein said method comprises the steps of:
i. identifying a nucleic acid sequence that is a potential target site for DNA-recombining enzymes that are capable to induce a site-specific DNA recombination of a sequence of interest in a genome, wherein said potential target site comprises two asymmetric recombinase target sites;
ii. providing a nucleic acid molecule encoding a first recombinase enzyme, wherein said first recombinase enzyme has been evolved by directed evolution or rational design to specifically recognize a first half-site of a recombinase target site;
iii. providing a nucleic acid molecule encoding a second recombinase enzyme, wherein said second recombinase enzyme has been evolved by directed evolution or rational design to specifically recognize a second half-site of a recombinase target site;
iv. providing a nucleic acid molecule encoding a linker peptide, wherein said nucleic acid encodes a linker oligopeptide comprising 6 to 30 amino acids;
v. creating an expression vector by cloning the nucleic acid molecule encoding a first recombinase enzyme, the nucleic acid molecule encoding a second recombinase enzyme and the nucleic acid molecule encoding a linker peptide into an expression vector;
vi. transfecting a cell, which comprises a DNA sequence, which is to be recombined, preferably inverted, with the expression vector of step v);
vii. expressing the fusion protein comprising a first recombinase enzyme, a second recombinase enzyme and a linker peptide;
viii. analyzing, whether the fusion protein expressed in step vii. is capable of inverting the DNA sequence on a human chromosome in said cell; and
ix. selecting a fusion protein which is capable of inverting the DNA sequence on a human chromosome in said cell according to step viii.

A nucleic acid sequence according to step i. that is a potential target site for DNA-recombining enzymes that are capable to induce a site-specific DNA recombination of a sequence of interest in a genome can be identified according to method described in WO 2018/229226 A1, which comprises for example the sub-steps of:
a) Screening the genome or a part thereof comprising the sequence of interest for two sequences that are potential spacer sequences, with a length of at least 5 and up to 12 bp, wherein one of the potential spacer sequence lies upstream of the sequence of interest and the other potential spacer sequence lies downstream of the sequence of interest and wherein the two sequences have a maximum distance of 2 megabases or less, preferably of 1.5 megabases or less or 1 megabases or less, more preferably of 900 kb, 800 kb, 700 kb, 600 kb or 500 kb, most preferably of 400 kb or 300 kb and a minimum distance of 150 bp,
b) Identifying potential target sites by determining for each potential spacer sequence the neighboring nucleotides, preferably 10 to 20 nucleotides, more preferably 12 to 15 nucleotides, most preferably 13 nucleotides on one side thereof form the potential first half site and the neighboring nucleotides, preferably 10 to 20 nucleotides, more preferably 12 to 15 nucleotides, most preferably 13 nucleotides, on the other side form the potential second half site, whereas both potential half sites and the spacer sequence in between form a potential target site,
c) The potential target sites identified in step b) are further screened to select for potential target sequences that do not occur (elsewhere) in the genome of the host to ensure a sequence specific recombination, preferably inversion.

Preferably, the sequences of the identified potential target sites for DNA-recombining enzymes are naturally occurring in the genome.

The first recombinase enzyme and the second recombinase enzyme according to steps ii. and iii. can be evolved by directed evolution or rational design, preferably e.g. by substrate linked directed evolution (SLiDE) as described in WO 2018/229226 A1, wherein said directed evolution comprises the steps of:
a) Selecting a nucleotide sequence upstream of the nucleotide sequence to be altered as first target site and a nucleotide sequence downstream of the nucleotide sequence to be altered as second target site, whereas the sequences of the target sites are preferably not identical, wherein each target site comprises a first half site and a second half site with each 10 to 20 nucleotides separated by a spacer sequence with 5 to 12 nucleotides,
b) Applying molecular directed evolution on at least one library of DNA-recombining enzymes using a vector comprising the first target site and the second target site as selected in a) as substrate,
until at least one first designer DNA-recombining enzyme is obtained that is active on the first target site and at least one second designer DNA-recombining enzyme is obtained that is active on the second target site as selected in a).

Selecting an asymmetric target site provides the opportunity to compare two different evolution strategies. A single recombinase can be evolved to recognize both 10 to 20 bp, more preferably 12 to 15 bp, most preferably 13 bp half-sites or two recombinases can be evolved in parallel for each half-site. Combining the two recombinases allows to form a functional heterodimer capable of recombining the asymmetric site.

In one embodiment of the invention, it is preferred to evolve a single recombinase to recognize both 10 to 20 bp, more preferably 12 to 15 bp, most preferably 13 bp half-sites.

In a further embodiment of the invention, it is preferred to evolve two recombinases in parallel for each half-site resulting in a heterodimer. Because the heterodimer consists of two recombinases, which can form either a heterodimer or two different homodimers, the amount of potential recognition sequences is increased. This approach may disadvantageously result in the increased chance of unintended recombination at off-target sites. To reduce the chance of recombination at off-target sites it was a further goal of the invention to constrain the monomers from homodimerization. To achieve this goal, the recombinase monomers were physically fused to enforce the desired heterodimer assembly, using a linker peptide. The linker peptide is generated by providing a nucleic acid molecule encoding a linker peptide, wherein said nucleic acid encodes a linker oligopeptide comprising 6 to 30 amino acids according to step iv. of the method of the invention. In order to enhance the activity of the fused recombinase monomers, there is preferably designed a linker library. Said linker library is suitably evolved for several, e.g. 10 cycles of SLiDE to find the most active linker variants. As a result, a final library is generated and fusion proteins are selected according to steps viii. and ix. of the method of the invention, which shows robust activity on the desired recombinase target site, indicating that variants with improved activity had been generated.

In order to select fusion proteins that are highly specific for a desired recombinase target site, i.e. fusion proteins that show a reduced off-target recombination, the method of the invention may comprise in a further embodiment the steps of
x. Identifying potential off-target sites of the desired fusion protein;
xi. Analyzing the recombinase activity of the desired fusion protein on the off-target sites identified in step x.; and
xii. Selecting fusion proteins that do not show recombinase activity on at least one off-target site.

In a preferred embodiment of the invention the C-terminus of the said second recombinase enzyme is connected to the N-terminus of said first recombinase enzyme via the linker peptide.

In a most preferred embodiment of the invention, the fusion protein expressed in step vii., the C-terminus of the said first recombinase enzyme is connected to the N-terminus of said second recombinase enzyme via the linker peptide.

Recombinase off-target sites can for example be identified using bioinformatics approaches known to the person skilled in the art. Other approaches include ChIP-Seq-based assays to identify putative off-targets in the human followed by validation and DNA enrichment by qPCR. These methods are also known to the person skilled in the art.

Recombinase activity of a fusion protein on these potential off-target sites can e.g. experimentally be tested by cloning the genomic sequences as excision substrates into a bacterial reporter vector, such as described herein below. Recombination at the off-target sites can then be detected by monitoring the expression of a reporter gene, e.g. using a PCR-based assay. Such an assay can also be performed in a human tissue culture to investigate whether an off-target site is altered by the fusion protein in vivo.

The method of the invention has the particular advantage that any recombinase target site can be used to evolve a recombinase fusion protein that shows a specific activity for this recombinase target site. Since the method of the invention includes the provision of a target site specific recombinase complex, wherein also the linker for interconnecting the monomers of recombinase heterodimers is specifically adapted to the evolved recombinases, the method of the invention has further the advantage that undesired off-target-activity of the recombinase complex, i.e. the recombinase fusion proteins, can be drastically reduced, preferably completely eliminated. This makes the recombinase complex, i.e. the recombinase fusion proteins especially suitable for use in gene therapies.

In another aspect, the invention provides a fusion protein for efficient and specific genome editing, comprising a complex of recombinases, wherein said complex of recombinases comprises at least a first recombinase enzyme, a second recombinase enzyme and at least one linker, wherein said first recombinase enzyme and said second recombinase enzyme specifically recognize a first half-site and a second half-site of a recombinase target site; wherein said first recombinase enzyme and said second recombinase enzyme are interconnected via a linker; and wherein said linker comprises or consists of an oligopeptide comprising 4 to 50 amino acids.

The recombinases comprised in the complex are preferably DNA recombinases and may be naturally occurring recombinases (i.e. recombinases isolated from any type of biologicals samples) or designer recombinases, such as recombinases evolved by directed molecular evolution or rational design, or any combinations thereof. Methods to create designer recombinases are known in the art. WO 2018/229226 A1 for example teaches vectors and methods to generate designer DNA recombining enzymes by directed molecular evolution. WO2008083931A1 discloses the directed molecular evolution of tailored recombinases (Tre 1.0) that uses sequences in the long terminal repeat (LTR) of HIV as recognition sites (loxLTR Tre 1.0). Further developments of this approach using asymmetric target sites were described in WO2011147590 A2 (Tre 3.0) and WO2016034553 A1 (Tre 3.1 and Tre/Brec1) as well as the publication Karpinski J et al 2016 (Brec1). Methods to engineer naturally occurring or designer-recombinases by rational design are also known to the art (e.g Abi-Ghanem et al., 2013; Karimova et al., 2016).

In a most preferred embodiment, the recombinases comprised in the complex were generated with the above described method of the invention. Accordingly, the features and embodiments relating to the recombinase target sites, fusion proteins, recombinases and linker peptides, which are subsequently described herein, also apply to the above described method of the invention.

Within the scope of the invention are compositions of the recombinase complex, wherein e.g.
all four recombinase monomers are different;
three recombinase monomers are identical and one monomer is different;
two recombinase monomers are identical and the other two monomers are different;
the complex comprises two different homodimers;
the complex comprises two different heterodimers;
the complex comprises two identical heterodimers;
all four recombinase monomers are identical;
the complex comprises two different monomers; or
the complex comprises two identical monomers.

“Different” in this context means that the monomers are not identical in their primary structure, i.e. show differences in their amino acid sequences; and/or show a high specificity towards one of the four half-sites of an upstream target site and a downstream target site of a recombinase, which leads advantageously to a surprisingly increased specificity of the fusion proteins of the invention.

In a preferred embodiment, the recombinase complex is a dimer, more preferably a heterodimer for recognition of a first target sequence and a second target sequence of an upstream or downstream recombinase target site in a DNA, wherein the monomers of said heterodimer are fused via a linker.

In a further preferred embodiment, the recombinase complex of the invention is a tetramer, more preferably a heterotetramer for the recognition of an upstream target site and a downstream target site of a recombinase in a DNA, wherein at least two monomers of said heterotetramer are fused via a linker.

In a more preferred embodiment, the complex consists of two heterodimers, most preferably two identical heterodimers, wherein the monomers of each heterodimer are interconnected via a linker.

Further preferably, the monomers of said heterodimer have been evolved by directed evolution or rational design to specifically recognize a first half-site or second half-site of a recombinase target site. Accordingly, a first heterodimer in said complex of two heterodimers, specifically recognizes the first half-site and the second half-site of an upstream recombinase target site in a DNA, wherein the second heterodimer specifically recognizes the first half-site and the second half-site of a downstream recombinase target site.

More preferably, the monomers of said heterodimer are tyrosine site-specific recombinases.

Most preferably, a first recombinase monomer of said heterodimer has an amino acid sequence characterized by the following specific amino acids:
Q in position 5, A in position 40, T in position 44, V in position 80, R in position 90, L in position 94, and R in position 219; or
Q in position 5, A in position 40, T in position 44, V in position 80, R in position 90, L in position 94, R in position 219, G in position 232, S in position 323 and L in position 325; or
Q in position 5, A in position 40, T in position 44, V in position 80, R in position 90, L in position 94, R at position 132, G at position 150, R in position 219, V in position 272, S in position 323 and L in position 325.

Further most preferably, a second recombinase monomer of said heterodimer has an amino acid sequence characterized by the following specific amino acids:
H in position 90, S in position 94, K in position 249, G in position 266, V in position 272 and K in position 282; or
Q in position 5, T in position 80, H in position 90, S in position 94, K in position 249, G in position 266, V in position 272 and K in position 282.

These amino acid positions refer to the numbering of the sequence of wild type recombinase Cre of SEQ ID NO: 68.

For the avoidance of doubt, when the recombinase complex is represented by a dimer, particularly a homodimer or preferably a heterodimer, one linker for the interconnection of the two monomers of the dimer is present in the fusion protein of the invention. When the recombinase complex is represented by a tetramer, one, two three or four linkers for the interconnection of the four monomers of the tetramer are present in the fusion protein of the invention.

Each of the linkers comprised in the fusion protein of the invention is suitably a peptide, preferably an oligopeptide. In a preferred embodiment, the linker consists of 4-50 amino acids, more preferably 5-40 amino acids, most preferably 6-30 amino acids.

Based on the general knowledge of the mechanism of action of recombinases and the steric conditions of the recombinase mechanism of action, it would have been surprising for the person skilled in the art that the fusion of recombinases would not inhibit recombination. Surprisingly, it was experimentally shown herein that a recombination event could be observed on a recombinase target site that was induced by binding of a fusion protein consisting of a heterodimer and a linker, which indicates an obligate fashion of the recombination event.

Accordingly, each linker comprised in a fusion protein of the invention is in one embodiment an oligopeptide comprising repeats of (G₂S) selected from the group consisting of

(SEQ ID NO: 1)
GGSGGS;
(SEQ ID NO: 2)
GGSGGSGGSGGS;
(SEQ ID NO: 3)
GGSGGSGGSGGSGGSGGS;
(SEQ ID NO: 4)
GGSGGSGGSGGSGGSGGSGGSGGS;
(SEQ ID NO: 5)
GGSGGSGGSGGSGGSGGSGGSGGSGGSGGS;
(SEQ ID NO: 6)
GGSGGSGGSGGSGGSGGSGGSGGSGGSGGSGGSGGS;
and
(SEQ ID NO: 7)
GGSGGSGGSGGSGGSGGSGGSGGSGGSGGSGGSGGSGGSGGS.

However, fusion of the recombinases with a linker comprising (G₂S) repeats resulted in some cases in a decrease of recombination activity compared to the non-linked recombinases. Further experiments have shown that the length of the linker plays a significant role on the activity of the recombinases. Good results were achieved with a linker comprising eight (G₂S) repeats. A further increase of the linker length did not improve recombination efficacy, however, decreased the specificity as the obtained fusion proteins showed activity on the symmetric sites of the recombinase target sites, which is not desired. Accordingly, in a preferred embodiment, the linker comprised in the fusion protein of the invention is most preferably an oligopeptide consisting of 24 amino acids.

It has been further investigated whether the amino acid composition of the linker influences recombination activity on the final target site. Three linker libraries were designed. The purpose of the libraries was to find a linker with the same high specificity on the symmetric sites (no recombination) and improved activity on the final loxF8 recombinase target site. Part or the whole linker sequence comprising eight (G₂S) repeats was changed to the degenerate codon RVM that codes for nine of the amino acids commonly used in natural linkers (Chen, et al., 2013), namely Ala, Arg, Asn, Asp, Glu, Gly, Lys, Ser, Thr amino acids. Thereby, a large number of linker variants were created.

In a further preferred embodiment, the fusion protein of the invention therefore comprises a linker, which comprises or consists of an oligopeptide with an amino acid sequence selected from formula 1, formula 2 and formula 3 as set out below, which represent the tree linker libraries mentioned above:

X₁-X₂-X₃-X₄-X₅-X₆-(G₂S)₄-X₇-X₈-X₉-X₁₀-X₁₁-X₁₂ (formula 1);

(G₂S)₂-X₁-X₂-X₃-X₄-X₅-X₆-X₇-X₈-X₉-X₁₀-X₁₁-X₁₂-(G₂S)₂ (formula 2); and

X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄ (formula 3);

wherein
G is glycine;
S is serine; and
each of X₁ to X₂₄ is independently selected from the group consisting of alanine, arginine, asparagine, aspartic acid, glutamine, glycine, lysine, serine and threonine.

In a preferred embodiment, said oligopeptide of formula 1, formula 2 or formula 3 does not consist of glycine and serine residues only.

With regard to the recombinases used in the recombinase complex, the fusion protein according to the invention preferably comprises complexes of heterodimers, wherein each recombinase enzyme of said heterodimers is a tyrosine site-specific recombinase, which have been evolved by directed evolution or generated by other means to independently recognize the half-sites of an upstream target site and the half-sites of a downstream target site of tyrosine site-specific recombinases.

Suitably, said tyrosine site-specific recombinases are selected from the group consisting of Cre, Dre-, VCre-, SCre-, Vika-, lambda-Int-, Flp-, R-, Kw-, Kd-, B2-, B3-, Nigri- and Panto-recombinase. The recognition target sites of these bacterial and yeast T-SSR systems have been discussed in Meinke et al., 2016 and in Karimova et al., 2016, and are shown in Table 1 below:

TABLE 1
Recognition Target Sites of Bacterial and Yeast T-SSR Systems

| T-SSR/Site | DNA target sequence (Natural host) |
|---|---|
| Cre/loxP | 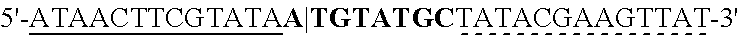 |
| | (SEQ ID NO: 73) (Bacteriophage P1) |
| Dre/rox | 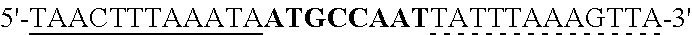 |
| | (SEQ ID NO: 74) (Bacteriophage P6) |
| VCre/Vloxp | 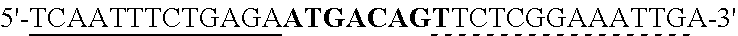 |
| | _(SEQ ID NO: 75) (P0908 of Vibrio sp.) |
| SCre/SloxP | 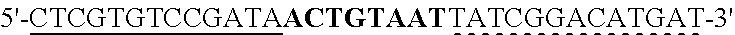 |
| | (SEQ ID NO: 76) (P1 of Shewanella sp.) |
| Vika/vox | 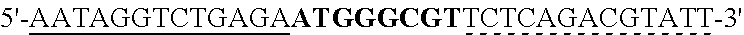 |
| | (SEQ ID NO: 77) (Vibrio coralliilyticus) |
| Lambda- | 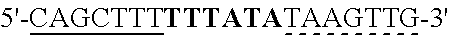 |
| Int/attP | (SEQ ID NO: 78) (Bacteriophage lambda) |
| FLP/FRT | 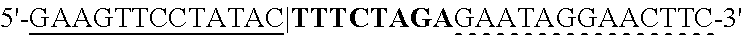 |
| | (SEQ ID NO: 79) (Saccharomyces cerevisiae) |
| R/RRT | 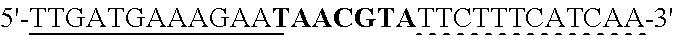 |
| | (SEQ ID NO: 80) (Zygosaccharomyces rouxii) |
| Kw/KwRT | 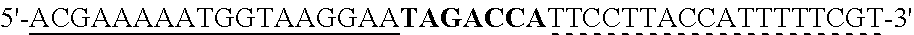 |
| | (SEQ ID NO: 81) (Kluyveromyces waltii) |
| Kd/KdRT | 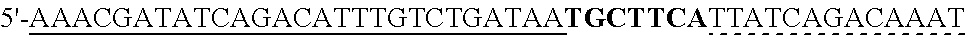 |
| | 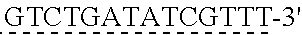 |
| | (SEQ ID NO: 82) (Kluyveromyces drosophilarum) |
| B2/B2RT | 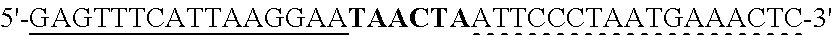 |
| | (SEQ ID NO: 83) (Zygosaccharomyces bailii) |
| B3/B3RT | 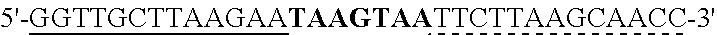 |
| | (SEQ ID NO: 84) (Zygosaccharomyces bisporus) |
| Nigri/nox | 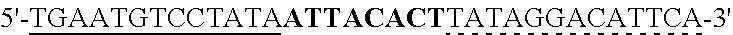 |
| | (SEQ ID NO: 85) (Vibrio nigripulchritudo) |
| Panto/pox | 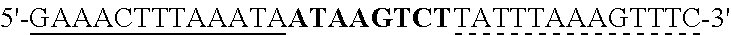 |
| | (SEQ ID NO: 86) (Pantoea sp. aB) |

Solid underlined: left half-site
Dashed-underlined: right half-site
Bold: Spacer sequence

As described above, a majority of severe cases of hemophilia A in patients are caused by the inversion due to intrachromosomal recombination with homologous regions outside of the F8 gene (Graw et al., 2005), such as the inversion of exon 1 due to homologous recombination between two 1 kb sequences, in intron 1 and in the region positioned telomeric to the F8 gene (FIG. 4).

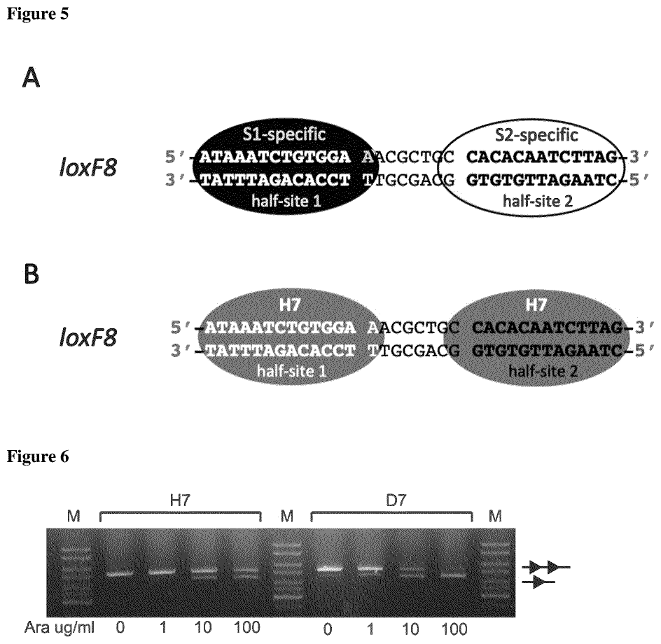

It was a further problem of the present invention to provide a fusion protein, which specifically corrects the DNA inversions, which are causing severe HA, preferably the inversion of exon 1. In order to solve this problem, two loxF8 target sites were identified in intron 1 and the homologous region positioned telomeric to the gene F8. Afterwards, two SSRs recognizing different half-sites of loxF8 were evolved by directed molecular evolution (FIG. 5A). Each of the so evolved recombinases recognizes one half-site of a final target site, and cooperatively perform the recombination.

By altering the DNA-binding properties of naturally occurring SSRs, these systems can be repurposed to recombine DNA sequences with therapeutic value. Methods to create designer recombinases are known in the art. WO 2018/229226 A1 for example teaches vectors and methods to generate designer DNA recombining enzymes by directed molecular evolution. WO2008083931A1 discloses the directed molecular evolution of tailored recombinases (Tre 1.0) that uses sequences in the long terminal repeat (LTR) of HIV as recognition sites (loxLTR Tre 1.0). Further developments of this approach using asymmetric target sites were described in WO2011147590 A2 (Tre 3.0) and WO2016034553 A1 (Tre 3.1 and Tre/Brec1) as well as the publication Karpinski J et al 2016 (Brec1). Methods to engineer naturally occurring or designer-recombinases by rational design are also known to the art (e.g. Abi-Ghanem et al., 2013; Karimova et al., 2016)

As an alternative to generating a single designer-recombinase that targets an asymmetric target site, dual designer-recombinases that recombine the target site together as a heterodimer can also be generated (Lansing et al., 2020). However, a comparative study for a single designer-recombinase and a dual designer-recombinase system (heterodimer), targeting the same DNA target has not been performed, yet. To perform such a comparative study, recombinases of the two classes utilizing SLiDE technology (Buchholz and Stewart, 2001; Lansing et al., 2020) were generated that were targeted to recombine a conserved sequence in the int1h1 and int1h2 sequences (loxF8, FIG. 4, FIG. 5).

To obtain a single recombinase recognizing the target sequence as a monomer, SLiDE was performed on the asymmetric loxF8 site, equivalent to the directed evolution of Tre and Brec on the asymmetric loxLTR and loxBRT sequences, respectively (Sarkar et al., 2007; Hauber et al., 2013). After 168 rounds of directed evolution, individual recombinases were tested in E. coli in the pEVO vector using different L-arabinose concentrations. The best clone identified (H7) recombined the loxF8 sequence when recombinase expression was induced with low concentrations of L-arabinose (FIG. 6).

To generate a recombinase system recognizing the same target sequence as a heterodimer, SLiDE was first performed on the symmetric loxF8 half-sites, equivalent to the directed evolution of the Hex recombinases, recombining a sequence on human chromosome 7 (Lansing et al., 2020). Recombinase libraries with activity on the symmetric loxF8 half-sites (after 88 and 89 rounds of directed evolution, respectively) were then coexpressed in E. coli from a pEVO-plasmid harboring two full, asymmetric loxF8 target sites. After 3 additional rounds of directed evolution, dual recombinase heterodimers were tested in E. coli in the pEVO vector using different L-arabinose concentrations. The best heterodimer clone identified (D7) recombined the loxF8 sequence more efficiently than the single H7 clone (FIG. 6), indicating that the generation of dual recombinase heterodimers is more effective than the generation of a single recombinase targeting an asymmetric site.

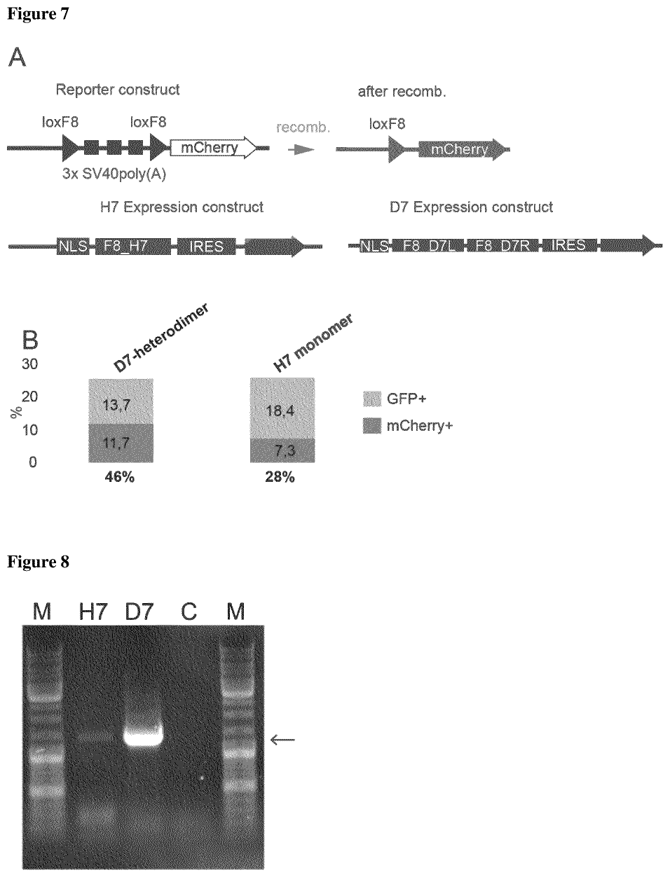

To investigate the recombination activities of the monomer (H7) and heterodimer (D7) F8-recombinases in mammalian cells, HeLa cells were co-transfected with recombination reporter and recombinase expression plasmids (FIG. 7A). As in the E. coli assays, the H7 recombinase was less efficient than the heterodimer D7 recombinase (FIG. 7B; 28% recombination efficiency versus 46% recombination efficiency).

To compare the abilities of the single F8-recombinase (H7) and the heterodimer F8 recombinase (D7) to induce the F8 inversion in human cells, the recombinase expression plasmids were transfected into HeLa cells. 48 hours post transfection genomic DNA was isolated. A PCR reaction was performed to detect the genomic inversion. Again, the monomer H7 recombinase did not perform as well as the heterodimer D7 recombinase in this assay (FIG. 8).

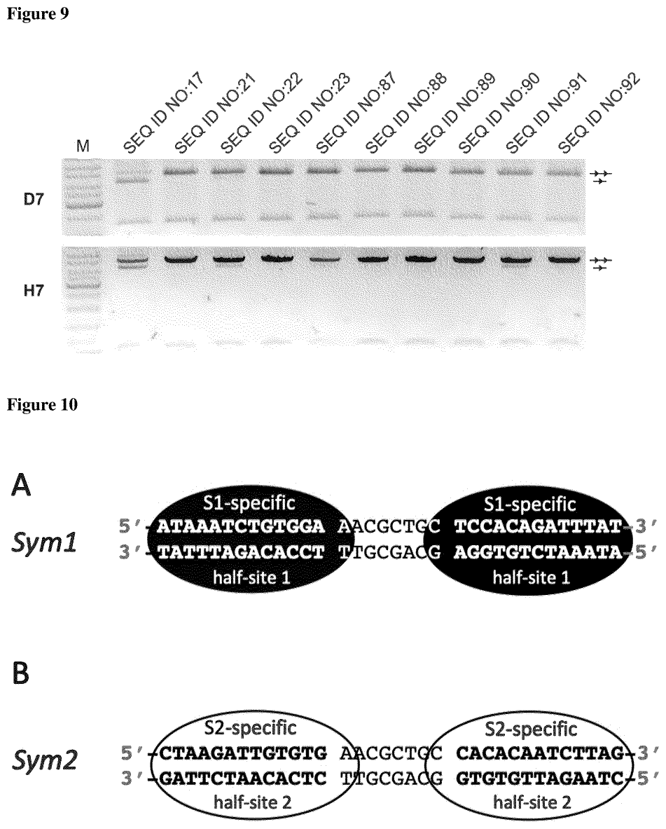

To compare the specificity of the H7 monomer with the D7 heterodimer, the recombinases were tested on nine human sequences that display the highest similarity to the loxF8 sequence (SEQ ID NOs: 21 to 23 and 87 to 92, Table 2). The nine off-target sites and the loxF8 on-target site were cloned as excision substrates into the pEVO vectors harboring the respective recombinases and the plasmids were grown for 24 hours in the presence of 10 μg/ml L-arabinose in E. coli. DNA extracted from these cultures revealed that the H7 monomer recombined the loxF8 site (SEQ ID NO: 17) in addition to the off-target sites with the SEQ ID NOs: 21, 22, 87 and 91. In contrast, the D7 heterodimer was more efficient in recombining the loxF8 sequence and also much more specific and only slightly recombined the off-target sites with the SEQ ID NO: 22 (FIG. 9). Based on these cumulative results, the heterodimer D7 was selected for further experiments.

While the D7 heterodimer showed better characteristics than the H7 monomer, it still showed some off-target recombination. For therapeutic applications it is desirable to employ genome editing tools that are highly specific and do not edit the genome at any other site then the intended one. As previous attempts have only reported limited success in improving the applied properties of heterospecific recombinases, possibly ways to improve the behavior of dual recombinases were explored. One possibility was to physically connect the two D7 heterodimers by fusing them with a peptide linker. State of the art literature suggested that this would most likely not work, as the individual enzymes need to move considerably during the recombination reaction. However, if it worked, this approach might provide a straightforward method for generating highly efficient and specific obligate recombination systems.

In a further embodiment of the invention, the recombinase heterodimer D11 has been evolved in the same manner as D7 heterodimer.

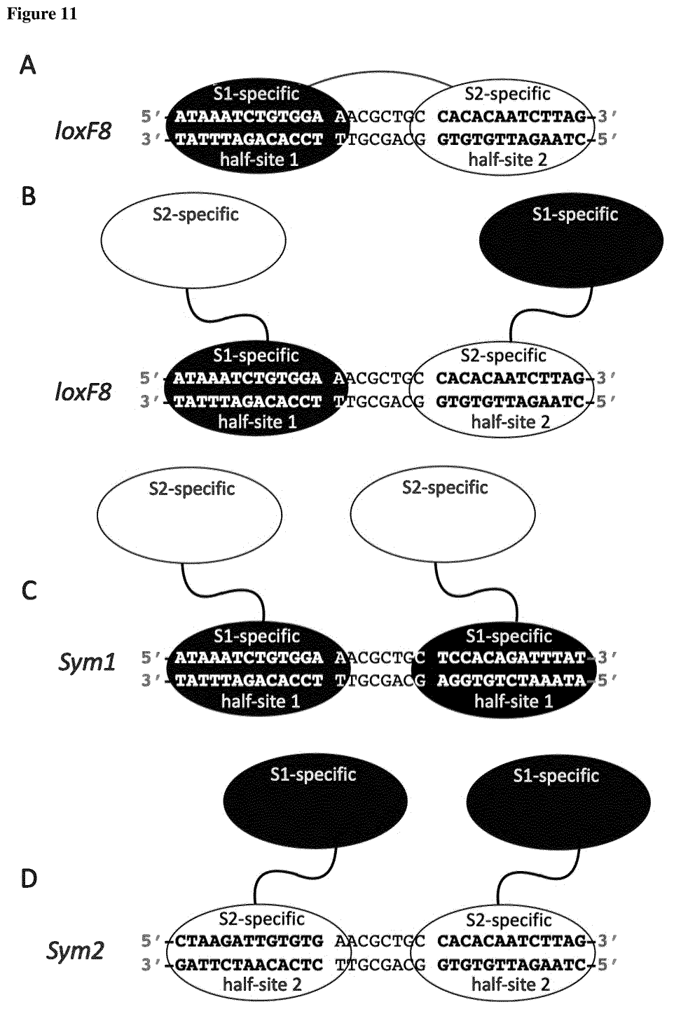

In a further preferred embodiment of the invention, the recombinase heterodimer A4-L has been evolved in the same manner as D7 heterodimer. In order to obtain the fused heterodimers with the best linker properties, they were selected for the activity on the loxF8 site and counter-selected for activity on both of the symmetric sites (FIG. 10). Linked recombinases can theoretically form different protein complexes with activity on the symmetric or asymmetric target sites (FIG. 11). All three initial libraries showed activity on the symmetric sites, indicating that the linker composition has an important influence on the specificity. Fusion proteins with linkers having an amino acid sequence selected from SEQ ID NOs. 8 to 16 showed a satisfying activity on the loxF8 site.

The invention therefore provides in a further embodiment a fusion protein, wherein said linker comprises or consists of an oligopeptide selected from the group consisting of

(SEQ ID NO: 8)
AEATSEGGSGGSGGSGGSNGARRT;
(SEQ ID NO: 9)
AGTTARGGSGGSGGSGGSGRRGAK;
(SEQ ID NO: 10)
KNGRGRGGSGGSGGSGGSRTKRET;
(SEQ ID NO: 11)
GGSGGSTAAKEGAASSASGGSGGS;
(SEQ ID NO: 12)
GGSGGSNSRSNTENSDKGGGSGGS;
(SEQ ID NO: 13)
GGSGGSNGEEGTERGKATGGSGGS;
(SEQ ID NO: 14)
GGSGGSTTKANRAKGGRGGGSGGS;
(SEQ ID NO: 15)
GANEDTNTEAAGSEGNEKTGTNSA;
and
(SEQ ID NO: 16)
GESRAEDGAKGNGRGKGEATAGAA.

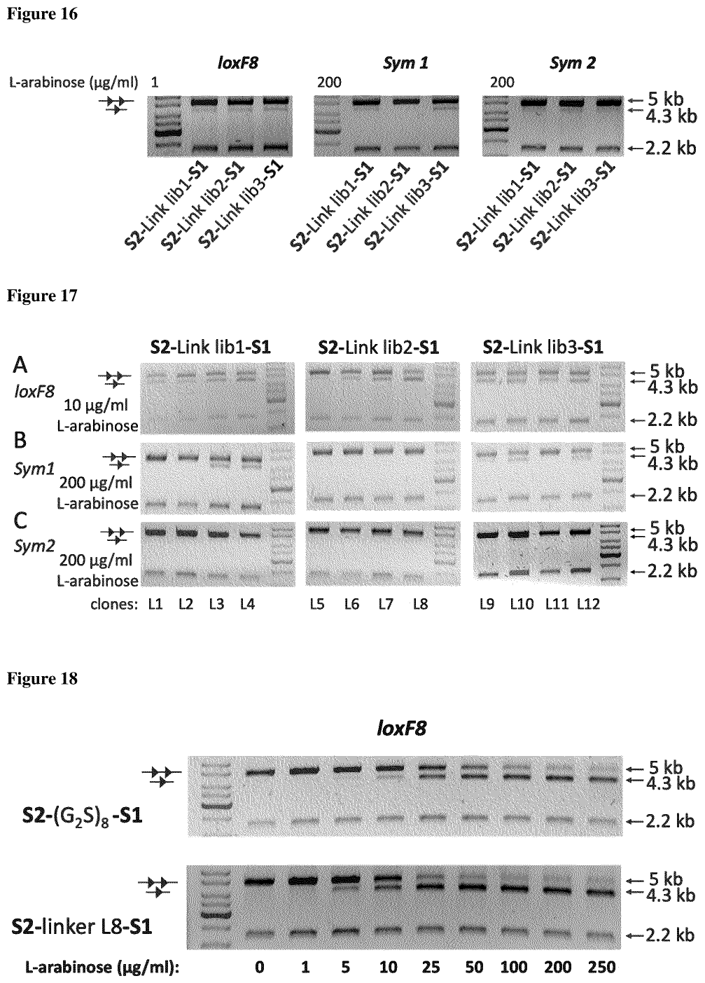

Further important criteria for the linker selection were the activity on the loxF8 site at low induction level and the absence of activity on the symmetric sites of loxF8 at high induction level (FIG. 16, FIG. 17). Best results in this regard were achieved with a fusion protein comprising a linker with the amino acid sequence

(SEQ ID NO: 14)
GGSGGSTTKANRAKGGRGGGSGGS.

Accordingly, in the preferred embodiment, the invention provides a fusion protein, wherein said linker comprises or consists of an oligopeptide, which has an amino acid sequence of SEQ ID NO: 14.

More preferably, the fusion protein of the invention specifically recognizes the upstream recombinase target sequence of the loxF8 target site, which has the nucleic acid sequence
ATAAATCTGTGGAAACGCTGCCACACAATCTTAG (SEQ ID NO: 17), or a reverse complement sequence thereof;
and that recognizes the downstream recombinase target sequence of the loxF8 target site, which has the nucleic acid sequence
CTAAGATTGTGTGGCAGCGTTTCCACAGATTTAT (SEQ ID NO: 18), or a reverse complement sequence thereof.

The capability of a fusion protein to catalyze the inversion of a DNA sequence between the upstream recombinase target sequence of SEQ ID NO. 17 and the downstream recombinase target sequence of SEQ ID NO: 18 of the loxF8 recombinase target site can be tested by a method comprising the steps of:
a) expressing the fusion protein comprising a first recombinase enzyme, a second recombinase enzyme and a linker peptide; and
b) analyzing, whether the fusion protein expressed in step a) is capable of inverting the DNA sequence on a human chromosome in said cell.

Most preferably, the fusion protein of the invention shows high specificity on the loxF8 target site with the target sequences of SEQ ID NOs: 17 and 18 and does not show activity on off-target sites at a high induction level (FIG. 20). Off-target sites, which are preferably not recognized by the fusion protein of the invention, are selected from the group consisting of SEQ ID NOs: 19 to 29 and 87 to 92 as shown in Table 2.

TABLE 2
Nucleic acid sequences of off-target sites

| Target | | SED ID |
|---|---|---|
| site | Sequence | NO: |
| loxF8 | ATAAATCTGTGGA AACGCTGC CACACAATCTTAG | 17 |
| Sym1 | ATAAATCTGTGGA AACGCTGC TCCACAGATTTAT | 19 |
| Sym2 | CTAAGATTGTGTG AACGCTGC CACACAATCTTAG | 20 |
| 1LR | ATAAATTTGTGGA AATTAAAC AACACACTCTTAA | 21 |
| 2LR | ACAAAATTGTGGA AATTAAAC AACACAATCTTAA | 22 |
| 3LR | ACAAATATGTGGA AATTAAAC AACACACTCTTGA | 23 |
| 4LR | ATAAATTTGTGGA AATTAAAC AACACACTCTTAA | 24 |
| 5LR | TTAAGAGTGTGTT TTTTAATT TCCACATATTTGT | 25 |
| 1L | ATAAATATGTGAA TATACATA TTCACATATTTAT | 26 |
| 2L | ATATATCTATAGA TATAGATA TCCACAGATATAT | 27 |
| 1R | CTATGTTTTTGAG GTCTTATT CACAAAATCTTTT | 28 |
| 2R | CTATTATTGTGTA ACAAATTA CCCCCAAACTTAG | 29 |
| HS4 | ATAAATATGTGTG TATATATA CACACAAACATAT | 87 |
| HS5 | ATATATCTGTGTA TATATATA CACACACACATAT | 88 |
| HS6 | ATATATATGTGTA TATATATA CACACATACATAT | 89 |
| HS7 | ATATATGTGTGTA TATATATA CACACACACATAC | 90 |
| HS8 | ATAAATATGTGTA AACTAAAC AACACACTCTTAA | 91 |
| HS9 | ACAAATATGTGGA AACTAAAC AACACATTCTTGA | 92 |

Bold: Sequences that are non-homologous to the target loxF8 sequence (SEQ ID NO: 17)
Underlined: Spacer sequences

The upstream (5′) recombinase target sequence of the loxF8 target site, which has the nucleic acid sequence of SEQ ID NO: 17; and the downstream (3′) recombinase target sequence of the loxF8 target site, which has the nucleic acid sequence SEQ ID NO: 18, have been identified as part of the invention. Accordingly, the invention relates in further preferred embodiment to a loxF8 recombinase target site comprising a 5′ target sequence of SEQ ID NO: 17 and a 3′ target sequence of SEQ ID NO: 17.

To generate DNA recombinases that recombine the upstream and downstream target sequences of the loxF8 target site, the substrate linked directed evolution approach was employed (Buchholz and Stewart 2001).

Said DNA recombinase according to the invention is an enzyme, which recombines a nucleic acid, in particular DNA, sequences by recognizing two target sites (recognition sites, i.e. one upstream and one downstream recognition site or sequence) and causing a deletion, an insertion, an inversion or a replacement of a DNA sequence. Advantageously, the DNA recombinase according to the invention recognizes the asymmetric recognition sites of the loxF8 sequence according to SEQ ID No. 17 (upstream) and SEQ ID NO: 18 (downstream). These recognition sites do not occur anywhere else in the human genome and therefore can be used for specific DNA recombination. The DNA recombinase according to the invention advantageously does not need target sites that are artificially introduced in the genome. Further advantageously and most preferably, the DNA recombinase according to the invention causes an inversion of a DNA sequence. A further advantage is that the DNA recombinase according to the invention allows precise genome editing without triggering endogenous DNA repair pathways.

Through several cycles of evolution, enzymes with activity on target sequences of SEQ ID NO: 17 and SEQ ID NO: 18 were generated.

The problem has firstly been solved by a recombinase monomer which has an amino acid sequence with at least 70%, preferably 80%, more preferably 90%, sequence identity with a sequence according to SEQ ID NO: 66 (recombinase H7). In a preferred embodiment, the invention therefore provides a recombinase monomer which has an amino acid sequence with at least 70%, preferably 80%, more preferably 90%, sequence identity with a sequence according to SEQ ID NO: 66.

In a preferred embodiment, the DNA recombinase of SEQ ID NO: 66 comprises one or more mutations compared to the Cre recombinase protein of SEQ ID NO: 68. Exemplary mutations include L5Q, V7L, P12S, P15L, V16A, V23T, M30V, F31L, R34S, H40Q, M44S, S47F, K57E, K62E, Y77H, Q90N, Q94S, S108G, T140A, D143S, Q144R, S147A, C155P, I166V, A175S, A231V, K244R, N245G, A249K, R259C, E262Q, E266A, T268A, 1272L, S305P, P307V, E308Q, N317T, N319E and I320S. More preferably, the recombinase of SEQ ID NO: 66 has, compared to the Cre recombinase protein of SEQ ID NO: 68, one or more, preferably two, three, for, five, six, eight, nine, ten or more mutations selected from the group consisting of include L5Q, V7L, P12S, P15L, V16A, V23T, M30V, F31L, R34S, H40Q, M44S, S47F, K57E, K62E, Y77H, Q90N, Q94S, S108G, T140A, D143S, Q144R, S147A, C155P, I166V, A175S, A231V, K244R, N245G, A249K, R259C, E262Q, E266A, T268A, I272L, S305P, P307V, E308Q, N317T, N319E and I320S. In a most preferred embodiment, the recombinase of SEQ ID NO: 66 has all of the aforementioned mutations.

For the avoidance of doubt, the amino acid which is indicated by the one letter code before the position number represents the amino acid in the wildtype sequence (for example SEQ ID NO: 68 of Cre recombinase); and the amino acid which is indicated by the one letter code after the position number represents the amino acid in the mutated, i.e. the evolved recombinase of the present invention.

Further most preferably, the sequence with at least 70%, preferably 80%, more preferably 90%, sequence identity with a recombinase of SEQ ID NO: 66 is characterized by the following specific amino acids: Q at position 5, Q at position 40, S at position 44, N at position 90, S at position 94, S at position 143, R at position 144, P at position 155, V at position 231, K at position 249, and L at position 272. These amino acid residues are predicted critical residues for target site recognition based on deep sequencing and are not found in the sequence of wildtype Cre-recombinase of SEQ ID NO: 68.

Because the recombination efficiency and specificity of this recombinase monomer was not satisfactory, DNA recombinase heterodimers were developed, also through several cycles of evolution.

As a result, the problem of the invention has been further solved by a DNA recombinase, which consists of a heterodimer, wherein said heterodimer comprises or consists of a first recombinase enzyme and a second recombinase enzyme, wherein said first recombinase enzyme is a polypetide, which has an amino acid sequence with at least 70%, preferably 80%, more preferably 90%, sequence identity with a sequence according to SEQ ID NO: 30 (recombinase 28-L) and wherein said second recombinase enzyme is a polypeptide, which has an amino acid sequence with at least 70%, preferably 80%, more preferably 90%, sequence identity with a sequence according to SEQ ID NO: 31 (recombinase 28-R).

Preferably, the DNA recombinase of SEQ ID NO: 30 comprises one or more mutations compared to the Cre recombinase protein of SEQ ID NO: 68. Exemplary mutations include N3D, L5Q, V7L, P12S, P15L, V23A, M30V, H40A, K43N, M44T, L58S, K62E, Y77H, A80V, K86N, Q90R, G93A, Q94L, S108G, A131V, K132R, E150G, N151S, C155R, Q156N, I166V, A175S, V182I, I195V, K219R, D232G, T253S, S257T, R259D, A260V, E262R, E266V, T268A, 1272V, Y273H, K276R, A285R, P307A, N317T, N319E, I320S, N323S and I325L. More preferably, the recombinase of SEQ ID NO: 30 has, compared to the Cre recombinase protein of SEQ ID NO: 68, one or more, preferably two, three, for, five, six, eight, nine, ten or more mutations selected from the group consisting of N3D, L5Q, V7L, P12S, P15L, V23A, M30V, H40A, K43N, M44T, L58S, K62E, Y77H, A80V, K86N, Q90R, G93A, Q94L, S108G, A131V, K132R, E150G, N151S, C155R, Q156N, 1166V, A175S, V182I, I195V, K219R, D232G, T253S, S257T, R259D, A260V, E262R, E266V, T268A, I272V, Y273H, K276R, A285R, P307A, N317T, N319E, I320S, N323S and I325L. In a most preferred embodiment, the recombinase of SEQ ID NO: 30 has all of the aforementioned mutations.

Further most preferably, the recombinase of SEQ ID NO: 30 exhibits a sequence characterized by the following specific amino acids: Q at position 5, A at position 40, T at position 44, V at position 80, R at position 90, L at position 94, R at position 155, R at position 219, V at position 266, V at position 272, S at position 323 and L at position 325. In a further preferred embodiment, the recombinase of SEQ ID NO: 30 exhibits a sequence, which is additionally characterized by the following specific amino acids: arginine at position 132, glycine at position 150, glycine at position 232 and arginine at position 276. These amino acid residues are predicted critical residues for target site recognition based on deep sequencing and are not found in the sequence of wildtype Cre-recombinase of SEQ ID NO: 68.

Preferably, the DNA recombinase of SEQ ID NO: 31 comprises one or more mutations compared to the Cre recombinase protein of SEQ ID NO: 68. Exemplary mutations include L5Q, T6A, V7L, L11P, P12S, A13T, P15L, V16A, S20C, R34S, M44A, L46Q, A53V, N60S, K62R, Y77H, A80T, Q90H, Q94S, R101Q, S108G, K122R, T140A, F142L, S147A, I166V, A175G, K183R, N235D, K244R, N245Y, A249K, R259Y, E262Q, E266G, T268A, 1272V, R282K, I306L, N317T, I320S and G342D. More preferably, the recombinase of SEQ ID NO: 31 has, compared to the Cre recombinase protein of SEQ ID NO: 68, one or more, preferably two, three, for, five, six, eight, nine, ten or more mutations selected from the group consisting of L5Q, T6A, V7L, L11P, P12S, A13T, P15L, V16A, S20C, R34S, M44A, L46Q, A53V, N60S, K62R, Y77H, A80T, Q90H, Q94S, R101Q, S108G, K122R, T140A, F142L, S147A, 1166V, A175G, K183R, N235D, K244R, N245Y, A249K, R259Y, E262Q, E266G, T268A, I272V, R282K, I306L, N317T, I320S, G342D. In a most preferred embodiment, the recombinase of SEQ ID NO: 31 has all of the aforementioned mutations.

Further most preferably, the sequence with at least 70%, preferably 80%, more preferably 90%, sequence identity with a recombinase of SEQ ID NO: 31 is characterized by the following specific amino acids: Q at position 5, A at position 44, T at position 80, H at position 90, S at position 94, K at position 249, G at position 266, V at position 272 and K at position 282. In a further preferred embodiment, the recombinase of SEQ ID NO: 31 exhibits a sequence, which is additionally characterized by the amino acids arginine at position 183. These amino acid residues are predicted critical residues for target site recognition based on deep sequencing and are not found in the sequence of wildtype Cre-recombinase of SEQ ID NO: 68.

Recombinant protein expression using bacterial and other host organisms is a fundamental technology for protein production. A key step in recombinant protein expression is codon optimization where a coding sequence for a protein of interest is designed by synonymous substitution aiming to increase its expression level. In conventional approach, rare codons are substituted by frequent codons according to the genomic codon usage in a host organism. The basis of this approach is that endogenous genes whose coding sequences consist of frequent codons have high protein expression levels, and thus recombinant protein expression is also considered to be improved by increasing the codon frequency. Another approach is to introduce synonymous substitution computationally predicted to destabilize mRNA secondary structures. Since stable mRNA secondary structures may inhibit translation, this approach is considered to improve recombinant protein expression by enhancing translational efficiency. The association between these sequence features and protein expression levels has been indicated by omics analyses of endogenous genes. The direct evidence of their influences in recombinant protein expression has been shown in the art using a relatively small number of genes.

After performing further cycles of molecular directed evolution, a DNA recombinase has been developed, which shows improved enzyme activity.

Accordingly, the invention provides in a further embodiment a DNA recombinase, which is a heterodimer, wherein the first monomer is recombinase enzyme which has a sequence with at least 70%, preferably 80%, more preferably 90% sequence identity with a sequence according to SEQ ID NO. 32 (recombinase D7-L) and wherein the second monomer is a recombinase enzyme which has a sequence with at least 70%, preferably 80%, more preferably 90%, sequence identity with a sequence according to SEQ ID NO. 33 (recombinase D7-R).

Preferably, the DNA recombinase of SEQ ID NO: 32 comprises one or more mutations compared to the Cre recombinase protein of SEQ ID NO: 68. Exemplary mutations include L5Q, V7L, P12S, P15L, V16A, D17N, V23A, M30V, Q35R, H40A, M44T, S51T, Y77H, A80V, K86N, Q90R, G93A, Q94L, S108G, N111S, A131V, K132R, Q144K, E150G, I166V, A175S, V182I, K219R, E222G, D232G, R259D, A260V, E262R, 1264V, E266A, T268A, I272V, A275T, R282G, A285T, P307A, N317T, N319E, I320S, N323S and I325L. In a more preferred embodiment, the recombinase of SEQ ID NO: 32 has, compared to the Cre recombinase protein of SEQ ID NO: 68, one or more, preferably two, three, for, five, six, eight, nine, ten or more mutations selected from the group consisting of L5Q, V7L, P12S, P15L, V16A, D17N, V23A, M30V, Q35R, H40A, M44T, S51T, Y77H, A80V, K86N, Q90R, G93A, Q94L, S108G, N111S, A131V, K132R, Q144K, E150G, I166V, A175S, V182I, K219R, E222G, D232G, R259D, A260V, E262R, 1264V, E266A, T268A, 1272V, A275T, R282G, A285T, P307A, N317T, N319E, I320S, N323S and I325L. In a most preferred embodiment, the recombinase of SEQ ID NO: 32 has all of the aforementioned mutations.

Further most preferably, the sequence with at least 70%, preferably 80%, more preferably 90%, sequence identity with a recombinase of SEQ ID NO: 32 is characterized by the following specific amino acids: Q at position 5, A at position 40, T at position 44, V at position 80, R at position 90, L at position 94, K at position 144, R at position 219, V at position 272, G at position 282, S at position 323 and L at position 325. In a further preferred embodiment, the recombinase of SEQ ID NO: 32 exhibits a sequence, which is additionally characterized by the following specific amino acids: arginine at position 132, glycine at position 150 and glycine at position 232. These amino acid residues are predicted critical residues for target site recognition based on deep sequencing and are not found in the sequence of wildtype Cre-recombinase of SEQ ID NO: 68.

Preferably, the DNA recombinase of SEQ ID NO: 33 comprises one or more mutations compared to the Cre recombinase protein of SEQ ID NO: 68. Exemplary mutations include L4I, L5Q, V7P, N10S, P12S, P15L, V16T, E22V, V23T, M28A, R34S, K57E, F64L, A66V, Y77H, A80T, Q90H, Q94S, S102A, S108G, K122R, K132Q, M149V, I166V, A175S, K244R, N245Y, A249K, R259Y, E262Q, E266G, T268A, I272V, D277G, R282K, S305P, N317T, I320S and G342S. In a more preferred embodiment, the recombinase of SEQ ID NO: 33 has, compared to the Cre recombinase protein of SEQ ID NO: 68, one or more, preferably two, three, for, five, six, eight, nine, ten or more mutations selected from the group consisting of L4I, L5Q, V7P, N10S, P12S, P15L, V16T, E22V, V23T, M28A, R34S, K57E, F64L, A66V, Y77H, A80T, Q90H, Q94S, S102A, S108G, K122R, K132Q, M149V, I166V, A175S, K244R, N245Y, A249K, R259Y, E262Q, E266G, T268A, I272V, D277G, R282K, S305P, N317T, I320S and G₃₄₂S. In a most preferred embodiment, the recombinase of SEQ ID NO: 33 has all of the aforementioned mutations.

Further most preferably, the sequence with at least 70%, preferably 80%, more preferably 90%, sequence identity with a recombinase of SEQ ID NO: 33 is characterized by the following specific amino acids: Q at position 5, T at position 80, H at position 90, S at position 94, K at position 249, G at position 266, V at position 272 and K at position 282. In a further preferred embodiment, the recombinase of SEQ ID NO: 33 exhibits a sequence, which is additionally characterized by the specific amino acids glutamine at position 132. These amino acid residues are predicted critical residues for target site recognition based on deep sequencing and are not found in the sequence of wildtype Cre-recombinase of SEQ ID NO: 68.

Preferably, the DNA recombinase monomer of SEQ ID NO: 32 exhibits a sequence, wherein the amino acid residue at position 5 is glutamine, the amino acid residue at position 17 is asparagine, the amino acid residue at position 35 is arginine, the amino acid residue at position 40 is alanine, the amino acid residue at position 44 is threonine, the amino acid residue at position 80 is valine, the amino acid residue at position 90 is arginine, the amino acid residue at position 94 is leucine, the amino acid residue at position 111 is serine, the amino acid residue at position 132 is arginine, the amino acid residue at position 144 is lysine, the amino acid residue at position 150 is glycine, the amino acid residue at position 219 is arginine, the amino acid residue at position 222 is glycine, the amino acid residue at position 264 is valine, the amino acid residue at position 272 is valine, the amino acid residue at position 275 is threonine, the amino acid residue at position 282 is glycine, the amino acid residue at position 323 is serine and/or the amino acid residue at position 325 is leucine.

These amino acid residues are specific for the DNA recombinase according to the invention and have not been found in the Cre recombinase (SEQ ID NO. 68).

Preferably, the DNA recombinase monomer of SEQ ID NO: 33 exhibits a sequence, wherein the amino acid residue at position 4 is isoleucine, the amino acid residue at position 5 is glutamine, the amino acid residue at position 7 is proline, the amino acid residue at position 16 is threonine, the amino acid residue at position 22 is valine, the amino acid residue at position 23 is threonine, the amino acid residue at position 28 is alanine, the amino acid residue at position 64 is leucine, the amino acid residue at position 66 is valine, the amino acid residue at position 80 is threonine, the amino acid residue at position 90 is histidine, the amino acid residue at position 94 is serine, the amino acid residue at position 102 is alanine, the amino acid residue at position 132 is glutamine, the amino acid residue at position 149 is valine, the amino acid residue at position 249 is Lysine, the amino acid residue at position 266 is glycine, the amino acid residue at position 272 is valine, the amino acid residue at position 277 is glycine, the amino acid residue at position 282 is lysine, the amino acid residue at position 305 is proline and/or the amino acid residue at position 342 is serine.

These amino acid residues are specific for the DNA recombinase according to the invention and have not been found in the Cre recombinase (SEQ ID NO. 68).

In a further preferred embodiment the invention provides a DNA recombinase, which is a heterodimer, wherein the first monomer is recombinase enzyme which has a sequence with at least 70%, preferably 80%, more preferably 90% sequence identity with a sequence according to SEQ ID NO. 93 (recombinase A4-L) and wherein the second monomer is a recombinase enzyme which has a sequence with at least 70%, preferably 80%, more preferably 90%, sequence identity with a sequence according to SEQ ID NO. 94 (recombinase A4-R).

Preferably, the DNA recombinase of SEQ ID NO: 93 comprises one or more mutations compared to the Cre recombinase protein of SEQ ID NO: 68. Exemplary mutations include N3S, L5Q, V7L, P12S, P15L, V23A, K25E, M28I, D29G, M30G, H40A, M44T, N60S, Y77H, A80V, K86N, Q90R, G93A, Q94L, S108G, A131V, Q144R, I166V, I174V, A175S, K211E, K219R, D232G, N257T, R259D, A260V, E262R, E266V, T268A, K276R, P307A, N317T, N319E, I320S, N323S and I325L. In a more preferred embodiment, the recombinase of SEQ ID NO: 93 has, compared to the Cre recombinase protein of SEQ ID NO: 68, one or more, preferably two, three, for, five, six, eight, nine, ten or more mutations selected from the group consisting of N3S, L5Q, V7L, P12S, P15L, V23A, K25E, M28I, D29G, M30G, H40A, M44T, N60S, Y77H, A80V, K86N, Q90R, G93A, Q94L, S108G, A131V, Q144R, I166V, I174V, A175S, K211E, K219R, D232G, N257T, R259D, A260V, E262R, E266V, T268A, K276R, P307A, N317T, N319E, I320S, N323S and I325L. In a most preferred embodiment, the recombinase of SEQ ID NO: 93 has all of the aforementioned mutations.

Further most preferably, the sequence with at least 70%, preferably 80%, more preferably 90%, sequence identity with a recombinase of SEQ ID NO: 93 is characterized by the following specific amino acids: Q at position 5, A at position 40, T at position 44, V at position 80, R at position 90, L at position 94, R at position 144, R at position 219, G at position 232, V at position 266, R at position 276, S at position 323 and L at position 325. These amino acid residues are predicted critical residues for target site recognition based on deep sequencing and are not found in the sequence of wildtype Cre-recombinase of SEQ ID NO: 68.

Preferably, the DNA recombinase of SEQ ID NO: 94 comprises one or more mutations compared to the Cre recombinase protein of SEQ ID NO: 68. Exemplary mutations include N3S, L5P, V7L, N10S, P12S, P15L, T19A, D21G, K25T, D29V, R34S, E39V, K57E, E67D, Y77H, Q90H, Q94S, N96D, S102A, S108G, K122R, E123A, K132G, I166V, A175S, K203R, K244R, N245Y, A249K, R259Y, E262Q, E266G, T268A, I272V, A275V, R282K, V304A, I306L, N317T, I320S and G342S. In a more preferred embodiment, the recombinase of SEQ ID NO: 94 has, compared to the Cre recombinase protein of SEQ ID NO: 68, one or more, preferably two, three, for, five, six, eight, nine, ten or more mutations selected from the group consisting of N3S, L5P, V7L, N10S, P12S, P15L, T19A, D21G, K25T, D29V, R34S, E39V, K57E, E67D, Y77H, Q90H, Q94S, N96D, S102A, S108G, K122R, E123A, K132G, I166V, A175S, K203R, K244R, N245Y, A249K, R259Y, E262Q, E266G, T268A, I272V, A275V, R282K, V304A, I306L, N317T, I320S and G342S. In a most preferred embodiment, the recombinase of SEQ ID NO: 94 has all of the aforementioned mutations.

Further most preferably, the sequence with at least 70%, preferably 80%, more preferably 90%, sequence identity with a recombinase of SEQ ID NO: 94 is characterized by the following specific amino acids: P at position 5, H at position 90, S at position 94, G at position 132, R at position 183, K at position 249, G at position 266, V at position 272 and K at position 282. These amino acid residues are predicted critical residues for target site recognition based on deep sequencing and are not found in the sequence of wildtype Cre-recombinase of SEQ ID NO: 68.

Preferably, the DNA recombinase monomer of SEQ ID NO: 93 exhibits a sequence, wherein the amino acid residue at position 3 is serine, the amino acid residue at position 5 is glutamine, the amino acid residue at position 25 is glutamic acid, the amino acid residue at position 28 is isoleucine, the amino acid residue at position 29 is glycine, the amino acid residue at position 30 glycine, the amino acid residue at position 40 is alanine, the amino acid residue at position 44 is threonine, the amino acid residue at position 60 is serine, the amino acid residue at position 80 is valine, the amino acid residue at position 90 is arginine, the amino acid residue at position 94 is leucine, the amino acid residue at position 144 is arginine, the amino acid residue at position 211 is glutamic acid, the amino acid residue at position 219 is arginine, the amino acid residue at position 232 is glycine, the amino acid residue at position 257 is threonine, the amino acid residue at position 266 is valine, the amino acid residue at position 276 is arginine, the amino acid residue at position 323 is serine and/or the amino acid residue at position 325 is leucine.

These amino acid residues are specific for the DNA recombinase according to the invention and have not been found in the Cre recombinase (SEQ ID NO. 68).

Preferably, the DNA recombinase monomer of SEQ ID NO: 94 exhibits a sequence, wherein the amino acid residue at position 3 is serine, the amino acid residue at position 5 is proline, the amino acid residue at position 21 is glycine, the amino acid residue at position 25 is threonine, the amino acid residue at position 29 is valine, the amino acid residue at position 39 is valine, the amino acid residue at position 67 is aspartic acid, the amino acid residue at position 90 is histidine, the amino acid residue at position 94 is serine, the amino acid residue at position 96 is aspartic acid, the amino acid residue at position 102 is alanine, the amino acid residue at position 123 is alanine, the amino acid residue at position 132 is glycine, the amino acid residue at position 183 is arginine, the amino acid residue at position 249 is Lysine, the amino acid residue at position 266 is glycine, the amino acid residue at position 272 is valine, the amino acid residue at position 275 is valine, the amino acid residue at position 282 is lysine, the amino acid residue at position 304 is alanine, the amino acid residue at position 306 is leucine and/or the amino acid residue at position 342 is serine.

These amino acid residues are specific for the DNA recombinase according to the invention and have not been found in the Cre recombinase (SEQ ID NO. 68).

In a further preferred embodiment the invention provides a DNA recombinase, which is a heterodimer, wherein the first monomer is recombinase enzyme which has a sequence with at least 70%, preferably 80%, more preferably 90% sequence identity with a sequence according to SEQ ID NO. 99 (recombinase D11-L) and wherein the second monomer is a recombinase enzyme which has a sequence with at least 70%, preferably 80%, more preferably 90%, sequence identity with a sequence according to SEQ ID NO. 100 (recombinase D11-R).

Preferably, the DNA recombinase of SEQ ID NO: 99 comprises one or more mutations compared to the Cre recombinase protein of SEQ ID NO: 68. Exemplary mutations include L5Q, V7I, P12T, L14S, P15L, V16A, V23A, M30V, F31L, H40A, M44T, S51T, L58S, Y77H, A80V, K86N, Q90R, G93A, Q94L, S108G, A131V, K132R, F142L, E150G, N151D, Q156K, L164P, I166V, A175S, V182I, K219R, A249V, R259D, A260V, E262R, E266A, A267T, T268A, I272V, K276R, D278G, Y283F, A285T, P307A, N317T, N319G and I320S. In a more preferred embodiment, the recombinase of SEQ ID NO: 99 has, compared to the Cre recombinase protein of SEQ ID NO: 68, one or more, preferably two, three, for, five, six, eight, nine, ten or more mutations selected from the group consisting of L5Q, V7I, P12T, L14S, P15L, V16A, V23A, M30V, F31L, H40A, M44T, S51T, L58S, Y77H, A80V, K86N, Q90R, G93A, Q94L, S108G, A131V, K132R, F142L, E150G, N151D, Q156K, L164P, I166V, A175S, V182I, K219R, A249V, R259D, A260V, E262R, E266A, A267T, T268A, I272V, K276R, D278G, Y283F, A285T, P307A, N317T, N319G and I320S. In a most preferred embodiment, the recombinase of SEQ ID NO: 99 has all of the aforementioned mutations.

Further most preferably, the sequence with at least 70%, preferably 80%, more preferably 90%, sequence identity with a recombinase of SEQ ID NO: 99 is characterized by the following specific amino acids: Q at position 5, A at position 40, T at position 44, V at position 80, R at position 90, L at position 94, R at position 132, G at position 150, R at position 219, V at position 272 and R at position 276. These amino acid residues are predicted critical residues for target site recognition based on deep sequencing and are not found in the sequence of wildtype Cre-recombinase of SEQ ID NO: 68.

Preferably, the DNA recombinase of SEQ ID NO: 100 comprises one or more mutations compared to the Cre recombinase protein of SEQ ID NO: 68. Exemplary mutations include N3D, L5Q, V7L, N10K, P12S, P15L, V16A, V23A, F31L, R34W, K57E, N60S, Q90H, Q94S, S108G, S147L, A175S, K183R, K211E, N235D, K244R, N245Y, A249K, R259Y, E262Q, E266G, T268A, I272V, R282K, S305P, N317T, N319G and I320S. In a more preferred embodiment, the recombinase of SEQ ID NO: 100 has, compared to the Cre recombinase protein of SEQ ID NO: 68, one or more, preferably two, three, for, five, six, eight, nine, ten or more mutations selected from the group consisting of N3D, L5Q, V7L, N10K, P12S, P15L, V16A, V23A, F31L, R34W, K57E, N60S, Q90H, Q94S, S108G, S147L, A175S, K183R, K211E, N235D, K244R, N245Y, A249K, R259Y, E262Q, E266G, T268A, 1272V, R282K, S305P, N317T, N319G and I320S. In a most preferred embodiment, the recombinase of SEQ ID NO: 100 has all of the aforementioned mutations.

Further most preferably, the sequence with at least 70%, preferably 80%, more preferably 90%, sequence identity with a recombinase of SEQ ID NO: 100 is characterized by the following specific amino acids: Q at position 5, H at position 90, S at position 94, R at position 183, K at position 249, G at position 266, V at position 272 and K at position 282. These amino acid residues are predicted critical residues for target site recognition based on deep sequencing and are not found in the sequence of wildtype Cre-recombinase of SEQ ID NO: 68.

Preferably, the DNA recombinase monomer of SEQ ID NO: 99 exhibits a sequence, wherein the amino acid residue at position 5 is glutamine, the amino acid residue at position 40 is alanine, the amino acid residue at position 44 is threonine, the amino acid residue at position 58 is serine, the amino acid residue at position 80 is valine, the amino acid residue at position 90 is arginine, the amino acid residue at position 94 is leucine, the amino acid residue at position 132 is arginine, the amino acid residue at position 142 is leucine, the amino acid residue at position 150 is glycine, the amino acid residue at position 156 is lysine, the amino acid residue at position 164 is proline, the amino acid residue at position 219 is arginine and/or the amino acid residue at position 319 is glycine.

These amino acid residues are specific for the DNA recombinase according to the invention and have not been found in the Cre recombinase (SEQ ID NO. 68).

Preferably, the DNA recombinase monomer of SEQ ID NO: 100 exhibits a sequence, wherein the amino acid residue at position 3 is aspartic acid, the amino acid residue at position 5 is glutamine, the amino acid residue at position 10 is lysine, the amino acid residue at position 34 is tryptophane, the amino acid residue at position 60 is serine, the amino acid residue at position 90 is histidine, the amino acid residue at position 94 is serine, the amino acid residue at position 183 is arginine, the amino acid residue at position 211 is glutamic acid, the amino acid residue at position 249 is lysine, the amino acid residue at position 266 is glycine, the amino acid residue at position 272 is valine, the amino acid residue at position 282 is lysine, the amino acid residue at position 305 is proline and/or the amino acid residue at position 319 is glycine.

These amino acid residues are specific for the DNA recombinase according to the invention and have not been found in the Cre recombinase (SEQ ID NO. 68).

The DNA recombinases according to the invention are site-specific recombinases that target a sequence present in the Int1h repeat sequence of the factor 8 gene, which is more preferably inverted by the activity of the DNA recombinases according to the invention.

The DNA recombinases of the present invention include the polypeptides of SEQ ID NOs.: 30 to 33, 93, 94, 99 and 100, as well as polypeptides which have at least 75% similarity (e.g. preferably at least 50%; and more preferably at least 70% identity) to a DNA recombinase of SEQ ID NOs.: 30 to 33, more preferably at least 85% similarity (e.g. preferably at least 70% identity) to a DNA recombinase of SEQ ID NOs.: 30 to 33, 93, 94, 99 and 100, and most preferably at least 95% similarity (e.g. preferably at least 90% identity) to a DNA recombinase of SEQ ID NOs.: 30 to 33. Moreover, they should preferably include exact portions of such DNA recombinases containing a sequence of at least 30 amino acids, and more preferably at least 50 amino acids. In a preferred embodiment, as used herein, the term “identity” refers to the amount of amino acids in accordance with one of the amino acid sequences SEQ ID NO.: 30, SEQ ID NO.: 31; SEQ ID NO.: 32, SEQ ID NO. 33, SEQ ID NO.: 93, SEQ ID NO.: 94, SEQ ID NO.: 99 or SEQ ID NO.: 100 with regard to the total number of amino acids.

Fragments or portions of the polypeptides of the present invention may be employed as intermediates for producing the corresponding full-length polypeptides by peptide synthesis. Fragments or portions of the polynucleotides of the present invention may also be used to synthesize full-length polynucleotides of the present invention.

In accordance with the present invention, activity and specificity of the DNA recombinases have been further increased by incorporating them in a fusion protein, wherein the monomers of said DNA recombinases are interconnected by a linker as described above.

Accordingly, the invention provides in a preferred embodiment a fusion protein, comprising a heterodimer of a DNA recombinase, wherein said heterodimer comprises a first recombinase enzyme, which has an amino acid sequence with at least 70%, preferably 80%, more preferably 90%, sequence identity with a sequence selected from SEQ ID NO: 30, 32, 93 or 99; and a second recombinase enzyme, which has the amino acid sequence with at least 70%, preferably 80%, more preferably 90%, sequence identity with a sequence selected from SEQ ID NO: 31, 33, 94 or 100 for the recognition of an upstream target sequence and a downstream target sequence of a recombinase target site.

In a more preferred embodiment, the invention provides a fusion protein, comprising a heterodimer of a DNA recombinase, wherein said heterodimer comprises a first recombinase enzyme, which has an amino acid sequence with at least 70%, preferably 80%, more preferably 90%, sequence identity with a sequence according to SEQ ID NO: 30 and a second recombinase enzyme, which has the amino acid sequence with at least 70%, preferably 80%, more preferably 90%, sequence identity with a sequence according to SEQ ID NO: 31 for the recognition of an upstream target sequence and a downstream target sequence of a recombinase target site.

In a further more preferred embodiment, the invention provides a fusion protein, comprising a heterodimer of a DNA recombinase, wherein said heterodimer comprises a first recombinase enzyme, which has an amino acid sequence with at least 70%, preferably 80%, more preferably 90%, sequence identity with a sequence according to SEQ ID NO: 99 and a second recombinase enzyme, which has the amino acid sequence with at least 70%, preferably 80%, more preferably 90%, sequence identity with a sequence according to SEQ ID NO: 100 for the recognition of an upstream target sequence and a downstream target sequence of a recombinase target site.

In a most preferred embodiment, the invention provides a fusion protein, comprising a heterodimer of a DNA recombinase, wherein said heterodimer comprises a first recombinase enzyme, which has an amino acid sequence with at least 70%, preferably 80%, more preferably 90%, sequence identity with a sequence according to SEQ ID NO: 32 and a second recombinase enzyme, which has the amino acid sequence with at least 70%, preferably 80%, more preferably 90%, sequence identity with a sequence according to SEQ ID NO: 33 for the recognition of an upstream target sequence and a downstream target sequence of a recombinase target site.

In a further most preferred embodiment, the invention provides a fusion protein, comprising a heterodimer of a DNA recombinase, wherein said heterodimer comprises a first recombinase enzyme, which has an amino acid sequence with at least 70%, preferably 80%, more preferably 90%, sequence identity with a sequence according to SEQ ID NO: 93 and a second recombinase enzyme, which has the amino acid sequence with at least 70%, preferably 80%, more preferably 90%, sequence identity with a sequence according to SEQ ID NO: 94 for the recognition of an upstream target sequence and a downstream target sequence of a recombinase target site.

As experimentally shown, the activity of the heterodimer—fusion protein is further influenced by the orientation, in which the recombinase monomers and the linker are connected to each other. Best activity of the heterodimer on the loxF8 site has been shown, when the first recombinase enzyme, the second recombinase enzyme and the linker are interconnected such that
i. the C-terminus of the recombinase enzyme with the amino acid sequence of SEQ ID NO: 31, which specifically recognizes a second half-site, such as the right half-site of a recombinase target site, is fused with the linker to the N-terminus of the recombinase enzyme with the amino acid sequence of SEQ ID NO: 30, which specifically recognizes a first half-site, such as the left half-site of a recombinase target site; and more preferably
ii. the C-terminus of the recombinase enzyme with the amino acid sequence of SEQ ID NO: 33, which specifically recognizes a second half-site, such as the right half-site of a recombinase target site, is fused with the linker to the N-terminus of the recombinase enzyme with the amino acid sequence of SEQ ID NO: 32, which specifically recognizes a first half-site, such as the left half-site of a recombinase target site.

Even best activity of the heterodimer on the loxF8 site has been shown, when the first recombinase enzyme, the second recombinase enzyme and the linker are interconnected such that
i. the C-terminus of the recombinase enzyme with the amino acid sequence of SEQ ID NO: 94, which specifically recognizes a second half-site, such as the right half-site of a recombinase target site, is fused with the linker to the N-terminus of the recombinase enzyme with the amino acid sequence of SEQ ID NO: 93, which specifically recognizes a first half-site, such as the left half-site of a recombinase target site; and more preferably
ii. the C-terminus of the recombinase enzyme with the amino acid sequence of SEQ ID NO: 100, which specifically recognizes a second half-site, such as the right half-site of a recombinase target site, is fused with the linker to the N-terminus of the recombinase enzyme with the amino acid sequence of SEQ ID NO: 99, which specifically recognizes a first half-site, such as the left half-site of a recombinase target site.

Accordingly, the complete fusion protein of the invention has in a preferred embodiment an amino acid sequence with at least 70%, preferably 80%, more preferably 90%, sequence identity with a sequence
i. according to SEQ ID NO: 71 or SEQ D NO.: 103; or most preferably
ii. according to SEQ ID NO: 72 or SEQ ID NO.: 97.

In a further embodiment, the invention provides a fusion protein, wherein the first recombinase enzyme, the second recombinase enzyme and the linker are interconnected such that
i. the C-terminus of the recombinase enzyme with the amino acid sequence of SEQ ID NO: 93, which specifically recognizes a first half-site, such as the left half-site of a recombinase target site, is fused with the linker to the N-terminus of the recombinase enzyme with the amino acid sequence of SEQ ID NO: 94, which specifically recognizes a second half-site, such as the right half-site of a recombinase target site; and more preferably
ii. the C-terminus of the recombinase enzyme with the amino acid sequence of SEQ ID NO: 99, which specifically recognizes a first half-site, such as the left half-site of a recombinase target site, is fused with the linker to the N-terminus of the recombinase enzyme with the amino acid sequence of SEQ ID NO: 100, which specifically recognizes a second half-site, such as the right half-site of a recombinase target site; and more preferably.

The invention further relates to a nucleic acid molecule, such as a polynucleotide or nucleic acid encoding a DNA recombinase or monomers thereof or a fusion protein according to the invention.

The “polynucleotides” or “nucleic acids” of the present invention may be in the form of RNA or in the form of DNA; DNA should be understood to include cDNA, genomic DNA, recombinant DNA and synthetic DNA. The DNA may be double-stranded or single-stranded and, if single stranded, may be the coding strand or non-coding (antisense) strand. The coding sequence, which encodes the polypeptide may be identical to the coding sequence for the polypeptides shown in SEQ ID NOs: 30 to 33, preferably of SEQ ID NOs: 32 and 33, or it may be a different coding sequence encoding the same polypeptide, as a result of the redundancy or degeneracy of the genetic code or a single nucleotide polymorphism. For example, it may also be an RNA transcript which includes the entire length of coding sequence for a polypeptide of any one of SEQ ID NOs 30 to 33, 93, 94, 99 and 100. In a preferred embodiment, the “polynucleotide” according to the invention is one of SEQ ID NOs 34 to 37, 67, 95, 96, 101 and 102 wherein the polynucleotide of SEQ ID NO: 34 encodes a DNA recombinase monomer of SEQ ID NO: 30; the polynucleotide of SEQ ID NO: 35 encodes a DNA recombinase monomer of SEQ ID NO: 31, the polynucleotide of SEQ ID NO: 36 encodes a DNA recombinase monomer of SEQ ID NO: 32, the polynucleotide of SEQ ID NO: 37 encodes a DNA recombinase monomer of SEQ ID NO: 33; the polynucleotide of SEQ ID NO: 67 encodes a DNA recombinase monomer of SEQ ID NO: 66. the polynucleotide of SEQ ID NO: 95 encodes a DNA recombinase monomer of SEQ ID NO: 93, the polynucleotide of SEQ ID NO: 96 encodes a DNA recombinase monomer of SEQ ID NO: 94, the polynucleotide of SEQ ID NO: 101 encodes a DNA recombinase monomer of SEQ ID NO: 99 and the polynucleotide of SEQ ID NO: 102 encodes a DNA recombinase monomer of SEQ ID NO: 100.

The nucleic acids which encode the polypeptides of SEQ ID NOs: 30 to 33, 66, 93, 94, 99 and 100, preferably of SEQ ID NOs: 32 and 33 or of SEQ ID NOs: 93 and 94 may include but are not limited to the coding sequence for the polypeptide alone; the coding sequence for the polypeptide plus additional coding sequence, such as a leader or secretory sequence or a proprotein sequence; and the coding sequence for the polypeptide (and optionally additional coding sequence) plus non-coding sequence, such as introns or a non-coding sequence 5′ and/or 3′ of the coding sequence for the polypeptide. The nucleic acids which encode the polypeptides of SEQ ID NOs: 30 to 33, 66, 93, 94, 99 and 100, preferably of SEQ ID NOs: 32 and 33 or of SEQ ID NOs: 93 and 94 include nucleic acids, which have been codon-optimized for expression in human cells. They may further contain a nuclear localization sequence.

Thus, the term “polynucleotide encoding a polypeptide” or the term “nucleic acid encoding a polypeptide” should be understood to encompass a polynucleotide or nucleic acid which includes only a coding sequence for a DNA recombinase enzyme of the invention, e.g. a polypeptide selected from SEQ ID NOs: 30 to 33, 66, 93, 94, 99 and 100, preferably of SEQ ID NOs: 32 and 33 or of SEQ ID NOs: 93 and 94 as well as one which includes additional coding and/or non-coding sequence. The terms polynucleotides and nucleic acid are used interchangeably.

The present invention also includes polynucleotides where the coding sequence for the polypeptide may be fused in the same reading frame to a polynucleotide sequence which aids in expression and secretion of a polypeptide from a host cell; for example, a leader sequence which functions as a secretory sequence for controlling transport of a polypeptide from the cell may be so fused. The polypeptide having such a leader sequence is termed a preprotein or a preproprotein and may have the leader sequence cleaved, by the host cell to form the mature form of the protein. These polynucleotides may have a 5′ extended region so that it encodes a proprotein, which is the mature protein plus additional amino acid residues at the N-terminus. The expression product having such a prosequence is termed a proprotein, which is an inactive form of the mature protein; however, once the prosequence is cleaved, an active mature protein remains. The additional sequence may also be attached to the protein and be part of the mature protein. Thus, for example, the polynucleotides of the present invention may encode polypeptides, or proteins having a prosequence, or proteins having both a prosequence and a presequence (such as a leader sequence).

The polynucleotides of the present invention may also have the coding sequence fused in frame to a marker sequence, which allows for purification of the polypeptides of the present invention. The marker sequence may be an affinity tag or an epitope tag such as a polyhistidine tag, a streptavidin tag, a Xpress tag, a FLAG tag, a cellulose or chitin binding tag, a glutathione-S transferase tag (GST), a hemagglutinin (HA) tag, a c-myc tag or a V5 tag.

The HA tag would correspond to an epitope obtained from the influenza hemagglutinin protein (Wilson et al., 1984), and the c-myc tag may be an epitope from human Myc protein (Evans et al., 1985).

If the nucleic acid of the invention is a mRNA, in particular for use as a medicament, the delivery of mRNA therapeutics has been facilitated by significant progress in maximizing the translation and stability of mRNA, preventing its immune-stimulatory activity and the development of in vivo delivery technologies. The 5′ cap and 3′ poly(A) tail are the main contributors to efficient translation and prolonged half-life of mature eukaryotic mRNAs. Incorporation of cap analogs such as ARCA (anti-reverse cap analogs) and poly(A) tail of 120-150 bp into in vitro transcribed (IVT) mRNAs has markedly improved expression of the encoded proteins and mRNA stability. New types of cap analogs, such as 1,2-dithiodiphosphate-modified caps, with resistance against RNA decapping complex, can further improve the efficiency of RNA translation. Replacing rare codons within mRNA protein-coding sequences with synonymous frequently occurring codons, so-called codon optimization, also facilitates better efficacy of protein synthesis and limits mRNA destabilization by rare codons, thus preventing accelerated degradation of the transcript. Similarly, engineering 3′ and 5′ untranslated regions (UTRs), which contain sequences responsible for recruiting RNA-binding proteins (RBPs) and miRNAs, can enhance the level of protein product. Interestingly, UTRs can be deliberately modified to encode regulatory elements (e.g., K-turn motifs and miRNA binding sites), providing a means to control RNA expression in a cell-specific manner. Some RNA base modifications such as N1-methyl-pseudouridine have not only been instrumental in masking mRNA immune-stimulatory activity but have also been shown to increase mRNA translation by enhancing translation initiation. In addition to their observed effects on protein translation, base modifications and codon optimization affect the secondary structure of mRNA, which in turn influences its translation. Respective modifications of the nucelic acid molecules of the invention are also contemplated by the invention.

The present invention is considered to further provide polynucleotides which hybridize to the hereinabove-described sequences wherein there is at least 70%, preferably at least 90%, and more preferably at least 95% identity or similarity between the sequences, and thus encode proteins having similar biological activity. Moreover, as known in the art, there is “similarity” between two polypeptides when the amino acid sequences contain the same or conserved amino acid substitutes for each individual residue in the sequence. Identity and similarity may be measured using sequence analysis software (e.g., ClustalW at PBIL (Pôle Bioinformatique Lyonnais) http://npsa-pbil.ibcp.fr). The present invention particularly provides such polynucleotides, which hybridize under stringent conditions to the hereinabove-described polynucleotides.

Suitably stringent conditions can be defined by, e.g., the concentration of salt or formamide in the prehybridization and hybridization solution, or by the hybridization temperature, and are well known in the art. In particular, stringency can be increased by reducing the concentration of salt, by increasing the concentration of formamide, and/or by raising the hybridization temperature.

For example, hybridization under high stringency conditions may employ about 50% formamide at about 37° C. to 42° C., whereas hybridization under reduced stringency conditions might employ about 35% to 25% formamide at about 30° C. to 35° C. One particular set of conditions for hybridization under high stringency conditions employs 42° C., 50% formamide, 5×SSPE, 0.3% SDS, and 200 μg/ml sheared and denatured salmon sperm DNA. For hybridization under reduced stringency, similar conditions as described above may be used in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art. Preferably, hybridization should occur only if there is at least 95%, and more preferably at least 97%, identity between the sequences. The polynucleotides which hybridize to the hereinabove described polynucleotides in a preferred embodiment encode polypeptides which exhibit substantially the same biological function or activity as the mature protein of SEQ ID NOs: 30 to 33, 66, 93, 94, 99 and 100, preferably of SEQ ID NOs: 32 and 33 or of SEQ ID NOs: 93 and 94.

As mentioned, a suitable polynucleotide probe may have at least 14 bases, preferably 30 bases, and more preferably at least 50 bases, and will hybridize to a polynucleotide of the present invention, which has an identity thereto, as hereinabove described. For example, such polynucleotides may be employed as a probe for hybridizing to the polynucleotides encoding the polypeptides of SEQ ID NOs: 30 to 33, 66, 93, 94, 99 and 100, such as to the polynucleotides of SEQ ID NOs: 34 to 37, 67, 95, 96, 101 and 102, respectively, for example, for recovery of such a polynucleotide, or as a diagnostic probe, or as a PCR primer. Thus, the present invention includes polynucleotides having at least a 70% identity, preferably at least a 90% identity, and more preferably at least a 95% identity to a polynucleotide of SEQ ID NOs: 34 to 37, 67, 95, 96, 101 and 102, which encodes a polypeptide of SEQ ID NOs: 30 to 33, 66, 93, 94, 99 and 100, as well as fragments thereof, which fragments preferably have at least 30 bases and more preferably at least 50 bases.

The terms “homology” or “identity,” as used interchangeably herein, refer to sequence similarity between two polynucleotide sequences or between two polypeptide sequences, with identity being a more strict comparison. The phrases “percent identity or homology” and “identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences.

A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantially identical,” as used herein, refers to an identity or homology of at least 70%, 75%, at least 80%, at least 85%, at least 90%, 91%, 92%, 93%, 94%, 95%, 96%, 97%, 98%, 99%, or more.

The degree of sequence identity is determined by choosing one sequence as the query sequence and aligning it with the internet-based tool ClustalW with homologous sequences taken from GenBank using the blastp algorithm (NCBI).

As it is well known in the art, the genetic code is redundant in that certain amino acids are coded for by more than one nucleotide triplet (codon), and the invention includes those polynucleotide sequences which encode the same amino acids using a different codon from that specifically exemplified in the sequences herein. Such a polynucleotide sequence is referred to herein as an “equivalent” polynucleotide sequence. The present invention further includes variants of the hereinabove described polynucleotides which encode for fragments, such as part or all of the protein, analogs and derivatives of a polypeptide of SEQ ID NOs 30 to 33, 66, 93, 94, 99 and 100. The variant forms of the polynucleotide may be a naturally occurring allelic variant of the polynucleotide or a non-naturally occurring variant of the polynucleotide. For example, the variant in the nucleic acid may simply be a difference in codon sequence for the amino acid resulting from the degeneracy of the genetic code, or there may be deletion variants, substitution variants and addition or insertion variants. As known in the art, an allelic variant is an alternative form of a polynucleotide sequence, which may have a substitution, deletion or addition of one or more nucleotides that does not substantially alter the biological function of the encoded polypeptide.

In are more preferred embodiment, the polynucleotide of the invention encodes a complete fusion protein of the invention, more preferably encodes a heterodimer of a DNA recombinase, wherein said heterodimer comprises a first recombinase enzyme, which has an amino acid sequence with at least 70%, preferably 80%, more preferably 90%, sequence identity with a sequence according to SEQ ID NO: 30, 32, 93 or 99 and a second recombinase enzyme, which has the amino acid sequence with at least 70%, preferably 80%, more preferably 90%, sequence identity with a sequence according to SEQ ID NO: 31, 33, 94 or 100 for the recognition of an upstream target sequence and a downstream target sequence of a recombinase target site; wherein said polynucleotide further encodes a linker as described herein.

In one embodiment, the polynucleotide of the invention comprises a nucleic acid of SEQ ID NO 34 and a nucleic acid of SEQ ID NO 35, and a nucleic acid encoding a linker oligopeptide.

In a further embodiment, the polynucleotide of the invention comprises a nucleic acid of SEQ ID NO 36 and a nucleic acid of SEQ ID NO 37, and a nucleic acid encoding a linker oligopeptide.

In a further embodiment, the polynucleotide of the invention comprises a nucleic acid of SEQ ID NO 95 and a nucleic acid of SEQ ID NO 96, and a nucleic acid encoding a linker oligopeptide.

In a further embodiment, the polynucleotide of the invention comprises a nucleic acid of SEQ ID NO 101 and a nucleic acid of SEQ ID NO 102, and a nucleic acid encoding a linker oligopeptide.

The nucleic acid encoding the linker oligopeptide is preferably a nucleic acid selected from SEQ ID NOs 38 to 54 (see Table 3 in Example 2)

More preferably, the nucleic acid encoding the linker oligopeptide is preferably a nucleic acid selected from SEQ ID NOs 55 to 63 (see Table 4 in Example 2).

Most preferably, the nucleic acid encoding the fusion protein of the invention is a nucleic acid comprising
i. nucleic acids with the sequence of SEQ ID NOs 34, 35 and 61; such as the nucleic acid of SEQ ID NO 64; or
ii. nucleic acids with the sequence of SEQ ID NOs 36, 37 and 61; such as the nucleic acid of SEQ ID NO 65; or
iii. nucleic acids with the sequence of SEQ ID NOs 93, 94 and 61; such as the nucleic acid of SEQ ID NO 98; or
iv. nucleic acids with the sequence of SEQ ID NOs 99, 100 and 61; such as the nucleic acid of SEQ ID NO 104; or
v. nucleic acids with the sequence of SEQ ID NOs 95 and 96; or
vi. nucleic acids with the sequence of SEQ ID NOs 101 and 102;
vii. nucleic acids with the sequence of SEQ ID NOs 34 and 35; or
viii. nucleic acids with the sequence of SEQ ID NOs 36 and 37; or
ix. the nucleic acid of SEQ ID NO: 67.

Most preferably, the nucleic acid encoding a fusion protein of the invention is a nucleic acid comprises or consists of SEQ ID NO 64.

Even most preferably, the nucleic acid encoding a fusion protein of the invention is a nucleic acid comprises or consists of SEQ ID NO 65.

Even most preferably, the nucleic acid encoding a fusion protein of the invention is a nucleic acid comprises or consists of SEQ ID NO 98.

Even most preferably, the nucleic acid encoding a fusion protein of the invention is a nucleic acid comprises or consists of SEQ ID NO 104.

The present invention also includes vectors, which include such polynucleotides, host cells which are genetically engineered with such vectors and the production of the polypeptides of SEQ ID NOs: 30 to 33, 66, 93, 94, 99 and 100 preferably of SEQ ID NOs: 32 and 33 or of SEQ ID NOs: 93 and 94 by recombinant techniques using the foregoing. Host cells are genetically engineered (transduced or transformed or transconjugated or transfected) with such vectors, which may be, for example, a cloning vector or an expression vector. The vector may be, for example, in the form of a plasmid, a conjugative plasmid, a viral particle, a phage, etc. The vector or the gene may be integrated into the chromosome at a specific or a not specified site. Methods for genome integration of recombinant DNA, such as homologous recombination or transposase-mediated integration, are well known in the art. The engineered host cells can be cultured in conventional nutrient media modified as appropriate for activating promoters, selecting transformants or amplifying the genes of the present invention. The culture conditions, such as temperature, pH and the like, are those commonly used with the host cell selected for expression, as well known to the ordinarily skilled artisan. The host cell can be a mammalian, insect, plant or bacterial host cell, comprising a nucleic acid or a recombinant polynucleotide molecule or an expression vector described herein.

The polynucleotide sequence in the expression vector is operatively linked to an appropriate expression control sequence(s) (promoter) to direct mRNA synthesis. As representative examples of such promoters, there may be mentioned: LTR or SV40 promoter, the E. coli lac, ara, rha or trp, the phage lambda PL promoter and other promoters known to control expression of genes in prokaryotic or eukaryotic cells or their viruses.

One skilled in the art can select a vector based on desired properties, for example, for production of a vector in a particular cell such as a mammalian cell or a bacterial cell.

Any of a variety of inducible promoters or enhancers can be included in the vector for expression of an antibody of the invention or nucleic acid that can be regulated. Such inducible systems, include, for example, tetracycline inducible System; metallothionein promoter induced by heavy metals; insect steroid hormone responsive to ecdysone or related steroids such as muristerone; mouse mammary tumor virus (MMTV) induced by steroids such as glucocorticoid and estrogen; and heat shock promoters inducible by temperature changes; the rat neuron specific enolase gene promoter; the human j-actin gene promoter; the human platelet derived growth factor B (PDGF-B) chain gene promoter; the rat sodium channel gene promoter; the human copper-zinc superoxide dismutase gene promoter; and promoters for members of the mammalian POU-domain regulatory gene family.

Regulatory elements, including promoters or enhancers, can be constitutive or regulated, depending upon the nature of the regulation. The regulatory sequences or regulatory elements are operatively linked to one of the polynucleotide sequences of the invention such that the physical and functional relationship between the polynucleotide sequence and the regulatory sequence allows transcription of the polynucleotide sequence. Vectors useful for expression in eukaryotic cells can include, for example, regulatory elements including the CAG promoter, the SV40 early promoter, the cytomegalovirus (CMV) promoter, the mouse mammary tumor virus (MMTV) steroid-inducible promoter, Pgtf, Moloney marine leukemia virus (MMLV) promoter, thy-1 promoter and the like.

If desired, the vector can contain a selectable marker. As used herein, a “selectable marker” refers to a genetic element that provides a selectable phenotype to a cell in which the selectable marker has been introduced. A selectable marker is generally a gene whose gene product provides resistance to an agent that inhibits cell growth or kills a cell. A variety of selectable markers can be used in the DNA constructs of the invention, including, for example, Neo, Hyg, hisD, Gpt and Ble genes, as described, for example in Ausubel et al., 1999 and U.S. Pat. No. 5,981,830. Drugs useful for selecting for the presence of a selectable marker include, for example, G418 for Neo, hygromycin for Hyg, histidinol for hisD, xanthine for Gpt, and bleomycin for Ble. DNA constructs of the invention can incorporate a positive selectable marker, a negative selectable marker, or both.

Various mammalian cell culture systems can also be employed to express a recombinant protein. Examples of mammalian expression systems include the COS-7 lines of monkey kidney fibroblasts. Other cell lines capable of expressing a compatible vector include, for example, the C127, 3T3, CHO, HeLa and BHK cell lines. Mammalian expression vectors will generally comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide required nontranscribed genetic elements.

The polypeptides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Recovery can be facilitated if the polypeptide is expressed at the surface of the cells, but such is not a prerequisite. Recovery may also be desirable of cleavage products that are cleaved following expression of a longer form of the polypeptide. Protein refolding steps as known in this art can be used, as necessary, to complete configuration of the mature protein. High performance liquid chromatography (HPLC) can be employed for final purification steps.

In accordance with a further embodiment of the invention, there are provided are gene therapy vectors, e.g., for use in systemically or locally increasing the expression of the fusion proteins of the invention in a subject. The gene therapy vectors find use in preventing, mitigating, ameliorating, reducing, inhibiting, and/or treating a disease that can be treated by genome editing, in particular of hemophilia A. The gene therapy vectors typically comprise an expression cassette comprising a polynucleotide encoding a fusion protein of the invention. In one embodiment, the vector is a viral vector. In a preferred embodiment, the viral vector is from a virus selected from the group consisting of adenovirus, retrovirus, lentivirus, herpesvirus and adeno-associated virus (AAV). In a more preferred embodiment, the vector is from one or more of adeno-associated virus (AAV) serotypes 1-11, or any subgroups or any engineered forms thereof. In another embodiment, the viral vector is encapsulated in an anionic liposome.

In another embodiment, the vector is a non-viral vector. In a preferred embodiment, the non-viral vector is selected from the group consisting of naked DNA, a cationic liposome complex, a cationic polymer complex, a cationic liposome-polymer complex, and an exosome.

If the vector is a viral vector, the expression cassette suitably comprises operably linked in the 5′ to 3′ direction (from the perspective of the mRNA to be transcribed), a first inverse terminal repeat, an enhancer, a promoter, the polynucleotide encoding a fusion protein of the invention, a 3′ untranslated region, polyadenylation (polyA) signal, and a second inverse terminal repeat. The promoter is e.g. selected from the group consisting of cytomegalovirus (CMV) promoter and chicken-beta actin (CAG) promoter. The polynucleotide comprises preferably DNA or cDNA or RNA or mRNA. In a preferred embodiment, the polynucleotide encoding a fusion protein of the invention comprises one or more of the polypeptides of SEQ ID NO: 30, SEQ ID NO: 31, SEQ ID NO: 32, SEQ ID NO: 33, SEQ ID NO: 66, SEQ ID NO: 93, SEQ ID NO: 94, SEQ ID NO: 99 and SEQ ID NO: 100. In a most preferred embodiment, the polynucleotide encoding a fusion protein of the invention has at least about 75%, 80%, 85% or 90% sequence identity, e.g. at least about 91%, 92%, 93%, 94%, 95%, 96%, 97%, 98%, 99% sequence identity, to one or more of SEQ ID NO: 64, SEQ ID NO: 65, SEQ ID NO: 67, SEQ ID NO: 98 and SEQ ID NO: 104.

The invention further relates to a fusion protein of the invention or a nucleic acid molecule, recombinant polynucleotide or expression vector of the invention for use as a medicament. In a more preferred embodiment, the invention further relates to a fusion protein of the invention or a nucleic acid molecule, recombinant polynucleotide or expression vector of the invention for use in the prevention or treatment of a disease that can be treated by genome editing, in particular hemophilia A.

In a further embodiment, the invention relates to use of a fusion protein of the invention or a nucleic acid molecule, recombinant polynucleotide or expression vector of the invention for the preparation of a medicament for the prevention or treatment of a disease that can be treated by genome editing, in particular hemophilia A.

In a further embodiment, the invention relates to a method of prevention or treatment of a disease that can be treated by genome editing, in particular hemophilia A, comprising the administering a therapeutically effective amount of a fusion protein of the invention or a nucleic acid molecule, recombinant polynucleotide or expression vector of the invention to a patient in need thereof.

The fusion protein of the invention or a nucleic acid molecule, recombinant polynucleotide or expression vector of the invention are particularly suitable for the treatment of severe forms of hemophilia A.

The fusion protein of the invention or a nucleic acid molecule, recombinant polynucleotide or expression vector of the invention can further be comprised in a pharmaceutical composition, which may optionally further contain one or more therapeutically acceptable diluents or carriers.

Provided are pharmaceutical compositions, e.g., for use in preventing or treating a disorder that can be treated by genome editing, such us hemophilia A, which comprises a therapeutically effective amount of a vector which comprises a nucleic acid sequence of a polynucleotide that encodes one or more fusion proteins according to the invention, or which comprises a therapeutically active amount of a nucleic acid encoding a fusion protein of the invention, or which comprises a therapeutically active amount of a recombinant fusion protein of the invention (together named “therapeutically active agents”).

It will be understood that the single dosage or the total daily dosage of the therapeutically active agents and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific nucleic acid or polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range per adult per day. The therapeutically effective amount of the therapeutically active agents, such as a vector according to the invention that should be administered, as well as the dosage for the treatment of a pathological condition with the number of viral or non-viral particles and/or pharmaceutical compositions described herein, will depend on numerous factors, including the age and condition of the patient, the severity of the disturbance or disorder, the method and frequency of administration and the particular peptide to be used.

The pharmaceutical compositions that contain a therapeutically active agent according to the invention may be in any form that is suitable for the selected mode of administration.

In one embodiment, a pharmaceutical composition of the present invention is administered parenterally.

The phrases “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

The therapeutically active agents of the invention can be administered, as sole active agent, or in combination with other active agents, in a unit administration form, as a mixture with conventional pharmaceutical supports, to animals and human beings.

In further embodiments, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

Solutions comprising the therapeutically active agents as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The therapeutically active agents can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be as solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions can be administered in a manner compatible with the dosage formulation and in such amount as is therapeutically effective. The formulations are easily administered in a variety of dosage forms, such as the type of injectable solutions described above, but drug release capsules and the like can also be employed. Multiple doses can also be administered. As appropriate, the therapeutically active agents described herein may be formulated in any suitable vehicle for delivery. For instance, they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but are not limited to water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes.

An appropriate therapeutic regimen can be determined by a physician, and will depend on the age, sex, weight, of the subject, and the stage of the disease. As an example, for delivery of a nucleic acid sequence encoding a fusion protein of the invention using a viral expression vector, each unit dosage of the fusion protein expressing vector may comprise 2.5 μl to 100 μl of a composition including a viral expression vector in a pharmaceutically acceptable fluid at a concentration ranging from 10¹¹ to 10¹⁶ viral genome per ml, for example.

The effective dosages and the dosage regimens for administering a fusion of the invention in the form of a recombinant polypeptide depend on the disease or condition to be treated and may be determined by the persons skilled in the art. An exemplary, non-limiting range for a therapeutically effective amount of a fusion protein of the present invention is about 0.1-10 mg/kg/body weight, such as about 0.1-5 mg/kg/body weight, for example about 0.1-2 mg/kg/body weight, such as about 0.1-1 mg/kg/body weight, for instance about 0.15, about 0.2, about 0.5, about 1, about 1.5 or about 2 mg/kg/body weight.

A physician or veterinarian having ordinary skill in the art may readily determine and prescribe the effective amount of the pharmaceutical composition required. For example, the physician or veterinarian could start doses of the therapeutically active agents of the invention employed in the pharmaceutical composition at levels lower than that required in order to achieve the desired therapeutic effect and gradually increase the dosage until the desired effect is achieved. In general, a suitable daily dose of a composition of the present invention will be that amount of the delivery system which is the lowest dose effective to produce a therapeutic effect. Such an effective dose will generally depend upon the factors described above. Administration may e.g. be intravenous, intramuscular, intraperitoneal, or subcutaneous, and for instance administered proximal to the site of the target. If desired, the effective daily dose of a pharmaceutical composition may be administered as two, three, four, five, six or more sub-doses administered separately at appropriate intervals throughout the day, optionally, in unit dosage forms. While it is possible for a delivery system of the present invention to be administered alone, it is preferable to administer the delivery system as a pharmaceutical composition as described above.

Further provided are kits comprising a therapeutically active agent as described above and herein. In one embodiment, the kits provide the therapeutically active agents prepared in one or more unitary dosage forms ready for administration to a subject, for example in a preloaded syringe or in an ampoule. In another embodiment, the therapeutically active agents are provided in a lyophilized form.

In a further embodiment, the invention provides a method for determining recombination on genomic level in a host cell culture, comprising a fusion protein for efficient and specific genome editing according to the invention, wherein said method comprises the steps of:
i. providing a nucleic acid molecule encoding a first recombinase enzyme, wherein said first recombinase enzyme has been evolved by directed evolution or rational design to specifically recognize a first half-site of a recombinase target site;
ii. providing a nucleic acid molecule encoding a second recombinase enzyme, wherein said second recombinase enzyme has been evolved by directed evolution or rational design to specifically recognize a second half-site of a recombinase target site;
iii. providing a nucleic acid molecule encoding a linker peptide, wherein said nucleic acid encodes a linker oligopeptide comprising 4 to 60 amino acids;
iv. creating an expression vector by cloning the nucleic acid molecule encoding a first recombinase enzyme, the nucleic acid molecule encoding a second recombinase enzyme and the nucleic acid molecule encoding a linker peptide into an expression vector which further comprises a first reporter gene for expression of a first reporter protein;
v. transfecting a host cell with the expression vector of step iv) and transfecting the host cell with a reporter plasmid comprising a second reporter gene for expressing a second reporter protein;
vi. expressing the fusion protein comprising a first recombinase enzyme, a second recombinase enzyme and a linker peptide, wherein said fusion protein is fused to the first reporter gene; and expressing the second reporter protein;
vii. identifying cells, which show a double expression of the first reporter protein and the second reporter protein, which is indicative for a successful recombination.

A suitable first reporter gene according to step iv. is the gene encoding for EGFP. Consequently, a suitable first reporter protein is EGFP.

A suitable second reporter gene according to step v. is the gene encoding for mCherry. Consequently, a suitable second reporter protein is mCherry.

This system has the advantage that the transfection efficiency of the cells transfected with both expression and reporter plasmid can be measured based on the GFP fluorescence. GFP and mCherry double positive cells reflect recombination of the reporter in human cells. In order to calculate the recombination efficiency of the reporter plasmid in human cells, the double positive cells can be normalized to the transfection efficiency.

The fusion proteins described herein were developed to correct a large gene inversion of exon 1 in the F8 gene which is causing Hemophilia A. To study the inversion efficacy of the heterodimers on genomic level, an in vitro recombinase assay as described in example 9 was developed. The inversion efficacy on genomic level in recombinase expressing cells was found to be 20.3% for the non-fused heterodimer and 42.7% for the fused heterodimer. Thus, the fusion of the recombinase heterodimer by a linker of the invention results in two-fold higher inversion rate.

Accordingly, the invention provides in a further embodiment a method for inversion of DNA sequence on genomic level in a cell, comprising a fusion protein or DNA recombinase for efficient and specific genome editing according to the invention, wherein said method comprises the steps of:
i. providing a nucleic acid molecule encoding a first recombinase enzyme, wherein said first recombinase enzyme has been evolved by directed evolution or rational design to specifically recognize a first half-site of a recombinase target site;
ii. providing a nucleic acid molecule encoding a second recombinase enzyme, wherein said second recombinase enzyme has been evolved by directed evolution or rational design to specifically recognize a second half-site of a recombinase target site;
iii. providing a nucleic acid molecule encoding a linker peptide, wherein said nucleic acid encodes a linker oligopeptide comprising 6 to 30 amino acids;
iv. creating an expression vector by cloning the nucleic acid molecule encoding a first recombinase enzyme, the nucleic acid molecule encoding a second recombinase enzyme and the nucleic acid molecule encoding a linker peptide into an expression vector;
v. delivering to a cell, which comprises a DNA sequence, which is to be inverted, an expression vector of step iv), a RNA molecule encoding a fusion protein decribed herein or a fusion protein described herein;
vi. inverting of the DNA sequence on a human chromosome in said cell.

Preferably, the fusion protein of step v. is a fusion protein of the invention as described herein, and more preferably recognizes the first half-site and the second half-site of an upstream target site and a downstream target site of a recombinase, most preferably the upstream target site of SQ ID NO: 17 and the downstream target site of SEQ ID NO: 18 of the loxF8 recombinase or a reverse complement sequence thereof.

In a further embodiment, said method of for inversion of DNA sequence on genomic level in a cell further comprises the step
v. a) expressing the fusion protein comprising a first recombinase enzyme, a second recombinase enzyme and a linker peptide;
   when in step v. of said method an expression vector of step iv. or a RNA molecule encoding a fusion protein as described herein is delivered into said cell, e.g. by transfection.

In one embodiment, the method for inversion of DNA sequence on genomic level is performed in genetically engineered host cell.

In a preferred embodiment, the method for inversion of DNA sequence on genomic level is performed in vitro in a human cell derived from a patient, more preferably from a patient suffering from hemophilia A.

In a further preferred embodiment, the method for inversion of DNA sequence on genomic level is performed in patients, in particular in a patient suffering from hemophilia A in vivo.

### EXAMPLES OF THE INVENTION

The following examples are provided for the sole purpose of illustrating various embodiments of the present invention and are not meant to limit the present invention in any fashion.

### Example 1: Substrate-Linked Directed Evolution

Recombinases were evolved using the previously described substrate-linked protein evolution (SLiDE) (Buchholz and Stewart, 2001; Sakara et al., 2007; Karpinski et al., 2016; Lansing, et al., 2019). By varying selection of active and inactive recombinases on the loxF8 and symmetric sites, respectively, a counterselection strategy was established.

### Example 2: Comparison of Recombination Activity of F8 Recombinase Monomer and F8 Non-Fused Recombinase Heterodimer

To compare whether the heterodimer recombinase, which consists of the monomers of SEQ ID NOs. 32 and 33, outperforms a single F8 monomer recombinase recognizing the loxF8 sequence, SLiDE with a single recombinase was performed on the asymmetric loxF8 site, equivalent to the directed evolution of Tre and Brec on the asymmetric loxLTR and loxBRT sequences, respectively (Sarkar et al., 2007; Hauber et al., 2013). After 168 rounds of directed evolution, individual recombinases were tested in E. coli in the pEVO vector using different arabinose concentrations. The best clone identified (H7, SEQ ID NO: 66) did recombine the loxF8 sequence, albeit at reduced efficiencies compared to the D7 heterodimer recombinase (SEQ ID NOs. 32 and 33) (FIG. 6).

To investigate the recombination activities of the monomer (H7) and heterodimer (D7) F8-recombinases in mammalian cells, HeLa cells were co-transfected with recombination reporter and recombinase expression plasmids. As in the E. coli assays, the H7 recombinase was less efficient than the heterodimer D7 recombinase (FIG. 7; 28% recombination efficiency versus 46% recombination efficiency).

To compare the abilities of the monomer F8-recombinase (H7) and the heterodimer F8 recombinase (D7) to induce the F8 inversion in human cells, the recombinase expression plasmids were transfected into HEK293 cells. 48 hours post transfection genomic DNA was isolated. A PCR reaction was performed to detect the genomic inversion. Again, the monomer H7 recombinase did not perform as well as the heterodimer D7 recombinase (FIG. 8).

To compare the specificity of the H7 monomer with the D7 heterodimer, the recombinases were tested on nine human sequences (SEQ ID NOs: 21 to 23 and 87 to 92) that display the highest similarity to the loxF8 sequence (SEQ ID NO: 17) (Table 2). These nine off-target sites and the loxF8 on-target site were cloned into the pEVO vectors harboring the respective recombinases and the plasmids were grown for 24 hours in the presence of 10 μg/ml L-arabinose in E. coli. DNA extracted from these cultures revealed that the H7 monomer recombined the loxF8 site in addition to the off-target sites HS1, HS2, HS4 and HS8, which have the sequences of SEQ ID NOs: 21, 22, 87 and 91, respectively. In contrast, the D7 heterodimer was much more specific and only slightly recombined HS2, while showing robust activity on the loxF8 on-target site (FIG. 9). Using the same experimental setting, a further highly active heterodimer (A4) was identified. Based on these cumulative results, the heterodimer D7 recombinase and the heterodimer A4 recombinase were selected for further experiments.

### Example 3: Recombination Activity of the Fused Heterodimer on the Symmetric Target Sites

Different linkers for fusing the F8 recombinase heterodimer were designed and tested. Flexible linkers with sequences (Gly-Gly-Ser)n and number of repeats (n) 2, 4, 6 and 8 were designed. The subsequent 5′ and 3′ oligonucleotides were synthesized with the addition of the sequences of “sticky ends” produced by XhoI and BsrGI-HF restriction enzymes, annealing of the sequences allowed obtaining the double-stranded DNA fragments that were directly used for cloning.

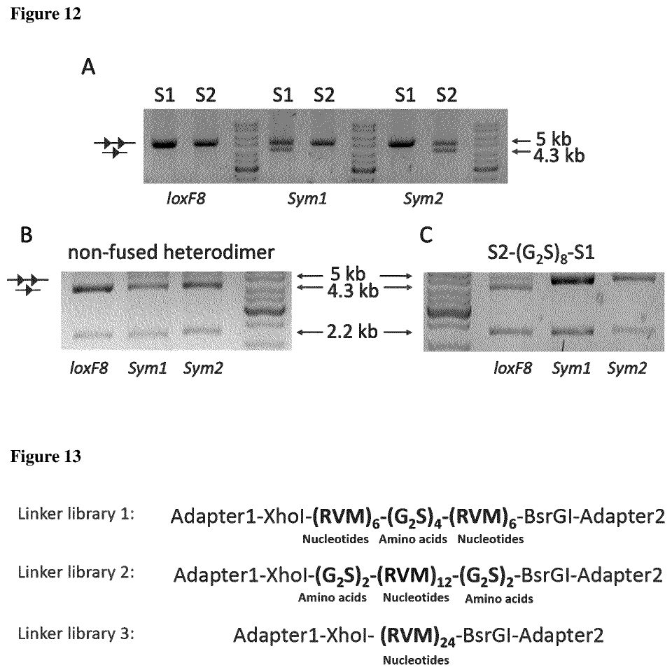

First, the non-fused heterodimer was tested on the symmetric sites. As expected, the test digest revealed strong recombination activity on the symmetric sites (FIG. 10, FIG. 12B), which can be explained by the activity of recombinase monomers on each of the symmetric sites. Importantly, however, the single recombinases do not show any activity on the final loxF8 site by themselves (FIG. 12A). On the other hand, the fused S2-(G₂S)₈-S1 heterodimer did not show any activity on the symmetric sites, even at high induction level (200 μg/ml L-arabinose) (FIG. 12C).

In order to compare the recombination activity of the fused heterodimer comparing to the non-fused one, both of them were tested at different induction levels. Afterwards, calculations of the recombination efficacy based on the band intensities was performed. Test digests revealed that the fused heterodimer has comparable activity to the non-fused one.

As it was shown from the results of the fusion with (G₂S)₂₋₈ linkers, the length of the linker influences the recombination activity. To study whether an even longer linker can increase the efficiency of the recombination without showing the activity on the symmetric sites, the heterodimer was fused with the linkers of 10, 12, and 14 (Gly-Gly-Ser) repeats. The results showed that increasing the linker length does not have a significant effect on recombination efficiency, however, the heterodimer fused with longer linkers showed activity on the symmetric sites. Therefore, recombinases fused with linkers of 30 or more amino acids result in specificity loss.

### Example 4: Linker Design and Generation of a Linker Library

Three linker libraries using degenerate codon RVM, coding for Ala, Arg, Asn, Asp, Glu, Gly, Lys, Ser, Thr amino acids, were designed. Linker length of 24 amino acids was used. The whole sequence (24 amino acids), middle part (12 amino acids) or the edges (6 amino acids from each side) of the (G₂S)₈ linker were changed to the RVM codon. Because it is not known that changes to the linker flexibility can influence the activity of the fused proteins, different options were considered while designing the libraries. The (G2S) sequence with a known flexibility property was kept either further from the recombinases (in the middle of the linker) or closer to the recombinases (on the edges of the linker) to investigate its influence on the activity. The first library was designed in a way that the middle part of the linker sequence (12 amino acids) was kept the same as in the initially used flexible linker, containing four (G2S) repeats, and the right and left parts of the linker sequence (6 amino acids from both sides) were changed to (RVM)₆. The second library was designed the other way around: the middle part of the 12 amino acids was changed to (RVM)₁₂, and the right and left parts of 6 amino acids were kept as two (G2S) repeats. In addition, as it was not known if the (G2S) repeats are needed in the linker, in the third library the whole 24 amino acids sequence of the linker was changed to (RVM)₂₄ (FIG. 13). Three oligonucleotides were designed and used as a template for three PCR reaction. The linker sequences were flanked by two adapters for primer annealing, and XhoI and BsrGI-HF restriction sites that allowed to use the linkers directly for cloning. The linker libraries were cloned in the fusion protein instead of (G2S)₈, resulting in three libraries: linker library 1, linker library 2 an linker library 3 (FIG. 13) The fusion proteins contained linker libraries, but the sequences of the recombinases were not changed.

Oligonucleotides with degenerate base modification, containing XhoI and BsrGI-HF restriction sites, and an adapter from each end, were synthesized by Biomers.net GmbH. Oligonucleotides sequences are shown in Table 3. The oligonucleotides were used as a template for high-fidelity PCR, the PCR products were subsequently digested with XhoI and BsrGI-HF restriction enzymes, and cloned into pEVO_2rec_link between the S2- and S1-specific recombinases. Freshly prepared competent E. coli XL-1 blue were transformed with the plasmids, containing the heterodimer fused with the library of linkers. After the incubation in SOC medium for 1 h, 2 μl (1:500) of the electroporation mixture was plated on an agar plate with chloramphenicol, the rest was added to 100 ml of LB medium with 25 μg/ml of chloramphenicol and desired concentration of L-Arabinose. Both, solid and liquid cultures were incubated at 37° C. for 12-16 h, the latter under constant shaking at 200 rpm. The library size was at least 25 000 linker variants that was assessed by the number of colonies on the plate.

TABLE 3
Sequences of the oligonucleotides used for linker generation

| | SEQ | |
|---|---|---|
| Name | ID NO | Sequence (5′→3′) |
| (Gly₂Ser)₂- | 38 | TCGAGGGTGGCAGCGGCGGTAGCAT |
| F | | |
| (Gly₂Ser)₂- | 39 | GTACATGCTACCGCCGCTGCCACCC |
| R | | |
| (Gly₂Ser)₄- | 40 | TCGAGGGCGGTAGCGGTGGCTCTGGCGGCAGCGGTGGTAG |
| F | | CAT |
| (Gly₂Ser)₄- | 41 | GTACATGCTACCACCGCTGCCGCCAGAGCCACCGCTACCGC |
| R | | CC |
| (Gly₂Ser)₆- | 42 | TCGAGGGCGGTAGCGGTGGCTCTGGCGGCAGCGGTGGTAG |
| F | | CGGTGGCTCT |
| | | GGCGGTAGTAT |
| (Gly₂Ser)₆- | 43 | TACATACTACCGCCAGAGCCACCGCTACCACCGCTGCCGCC |
| R | | AGAGCCACC |
| | | GCTACCGCCC |
| (Gly₂Ser)₈- | 44 | TCGAGGGCGGTAGCGGTGGCTCTGGCGGCAGCGGTGGTAG |
| F | | CGGTGGCTCT |
| | | GGCGGTAGTGGTGGTAGTGGCGGCA |
| (Gly₂Ser)₈- | 45 | GTACATGCTGCCGCCACTACCACCACTACCGCCAGAGCCAC |
| R | | CGCTACCAC |
| | | CGCTGCCGCCAGAGCC |
| (Gly₂Ser)₁₀- | 46 | TCGAGGGCGGTAGCGGTGGCTCTGGCGGCAGCGGTGGTAG |
| F | | CGGTGGCTCT |
| | | GGCGGTAGTGGTGGTAGTGGCGGCAGCGGTGGCTCTGGCG |
| | | GTAGTAT |
| (Gly₂Ser)₁₀- | 47 | GTACATACTACCGCCAGAGCCACCGCTGCCGCCACTACCAC |
| R | | CACTACCGCCAGAGCCACCGCTACCACCGCTGCCGCCAGA |
| | | GCCACCGCTACCGCCC |
| (Gly₂Ser)₁₂- | 48 | TCGAGGGCGGTAGCGGTGGCTCTGGCGGCAGCGGTGGTAG |
| F | | CGGTGGCTCTGGCGGTAGTGGTGGTAGTGGCGGCAGCGGT |
| | | GGCTCTGGCGGTAGTGGTGGTAGCGGTGGCTCTAT |
| (Gly₂Ser)₁₂- | 49 | GTACATAGAGCCACCGCTACCACCACTACCGCCAGAGCCA |
| R | | CCGCTGCCGCCACTACCACCACTACCGCCAGAGCCACCGCT |
| | | ACCACCGCTGCCGCCAGAGCCACCGCTACCGCCC |
| (Gly₂Ser)₁₄- | 50 | TCGAGGGCGGTAGCGGTGGCTCTGGCGGCAGCGGTGGTAG |
| F | | CGGTGGCTCTGGCGGTAGTGGTGGTAGTGGCGGCAGCGGT |
| | | GGCTCTGGCGGTAGTGGTGGTAGCGGTGGCTCTGGAGGTA |
| | | GTGGCGGATCAAT |
| (Gly₂Ser)₁₄- | 51 | GTACATTGATCCGCCACTACCTCCAGAGCCACCGCTACCAC |
| R | | CACTACCGCCAGAGCCACCGCTGCCGCCACTACCACCACTA |
| | | CCGCCAGAGCCACCGCTACCACCGCTGCCGCCAGAGCCAC |
| | | CGCTACCGCCC |
| Linker | 52 | GAAGTGCCATTCCGCCTGACCTCTCGAGRVMRVMRVMRV |
| library 1 | | MRVMRVMGGCGGCAGCGGTGGTAGCGGTGGCTCTGGCGG |
| | | TAGTRVMRVMRVMRVMRVMRVMATGTACAAGGCTAGGT |
| | | GGAGGCTCAGTG |
| Linker | 53 | GAAGTGCCATTCCGCCTGACCTCTCGAGGGCGGTAGCGGT |
| library 2 | | GGCTCTRVMRVMRVMRVMRVMRVMRVMRVMRVMRVMR |
| | | VMRVMGGTGGTAGTGGCGGCAGCATGTACAAGGCTAGGT |
| | | GGAGGCTCAGTG |
| Linker | 54 | GAAGTGCCATTCCGCCTGACCTCTCGAGRVMRVMRVMRV |
| library 3 | | MRVMRVMRVMRVMRVMRVMRVMRVMRVMRVMRVMRV |
| | | MRVMRVMRVMRVMRVMRVMRVMRVMATGTACAAGGCT |
| | | AGGTGGAGGCTCAGTG |

### Example 5: Linker Selection

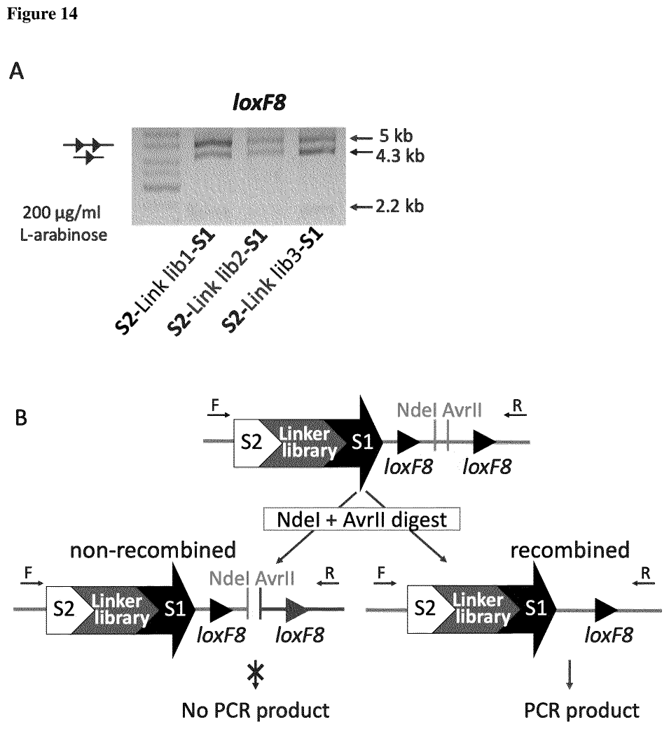

The fusion proteins with linker libraries were subcloned to the pEVO_2rec_loxF8 plasmid in order to test their activity on the loxF8 target site. Test digest revealed that the heterodimers fused with all three linker libraries have activity on the loxF8 site at high induction level of 200 μg/ml L-arabinose (FIG. 14A).

In order to select for heterodimers fused with linkers that have recombination activity on the loxF8 site, first, digestion with NdeI and AvrII restriction enzymes was performed. The restriction sites of these enzymes are located between the two loxF8 sites on the pEVO_2rec_LoxF8 plasmid, therefore, only the plasmids that were not recombined, were linearized. Afterwards, high-fidelity PCR was performed with the forward primer (primer 4) annealing upstream the fusion protein, and the reverse primer (primer 19) annealing downstream the second loxF8 site. As a result, only the fusion proteins that have recombined the plasmid were amplified by the PCR, because the non-recombined plasmids are digested and amplification is interrupted (FIG. 14B).

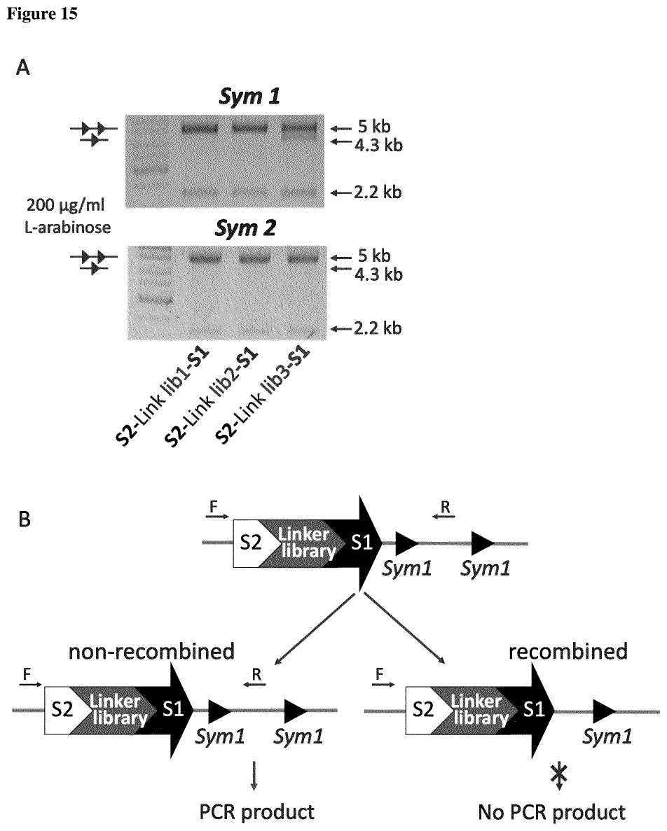

The obtained PCR product was subsequently digested with SacI-HF and SbfI-HF restriction enzymes and subcloned to the pEVO_2rec_Sym1 plasmid in order to test the activity on the Sym1 site. Test digest revealed that only one of the libraries, S2-link lib3-S1, had activity on the Sym1 site at high induction level of 200 μg/ml L-arabinose (FIG. 15A). The activity of the heterodimer fused with the linker library 1 and 2 were not detected by the test digest. In order to select the fused heterodimers that do not recombine the Sym1 site, high-fidelity PCR was performed with the forward primer (primer 20) annealing upstream the fusion protein, and the reverse primer (primer 21) annealing between two Sym1 sites (FIG. 15B). Therefore, only fusion proteins that did not recombine the plasmid were amplified by the PCR. The obtained PCR product was digested with SacI-HF and SbfI-HF restriction enzymes and subcloned to the pEVO_2rec_Sym2 plasmid in order to test the activity on the Sym2 site. No strong activity was detected by the test digest on the Sym2 site at high induction level of 200 μg/ml L-arabinose, and the selection was performed the same way as on the Sym1 site (FIG. 15).

To select the linkers with the best properties, the same steps were repeated and the following selection rounds were performed: on loxF8 at 10 μg/ml L-arabinose, Sym1 at 200 μg/ml L-arabinose, Sym2 at 200 μg/ml L-arabinose, loxF8 at 1 μg/ml L-arabinose, Sym1 at 200 μg/ml L-arabinose, Sym2 at 200 μg/ml L-arabinose, loxF8 at 1 μg/ml L-arabinose. Lower induction levels were used during the selection on the loxF8 site to select fusion proteins with high activity on the final target site. During the selection on the symmetric sites, however, L-arabinose concentration was kept high at 200 μg/ml to select fusion protein without activity on the symmetric sites. After 10 selection rounds all three fusion proteins with the linker libraries showed activity on the loxF8 site at low induction level of 1 μg/ml L-arabinose. However, the activity of the libraries on the Sym1 and Sym2 sites at high induction level of 200 μg/ml L-arabinose had also increased (FIG. 16). The heterodimers fused with all three linker libraries had activity on the Sym2 site, and the heterodimers fused with the linker library 1 and 3 had activity on the Sym1 site. The increase of the activity on the symmetric sites is likely due to the accumulation of the active heterodimers in the libraries.

Because the libraries showed activity on the loxF8 site at the very low induction level, at this point, single clones were analysed. Even though the libraries had some activity on the symmetric sites, it was expected that single linked recombinases from those libraries might only recombine loxF8 but not Sym1 or Sym2.

After testing the libraries on the loxF8 site at 1 μg/ml L-arabinose, the digestion with NdeI and AvrII restriction enzymes was performed and the plasmids that were not digested were retransformed. That allowed to enrich the libraries for the fused proteins that are active at low induction level. Then the fusion proteins with linker libraries were again subcloned to the pEVO_2rec_LoxF8 plasmid and grown on a plate. 32 colonies of each library were picked, induced with 10 μg/ml L-arabinose, and tested for recombination using a three-primer PCR assay. 90 clones displayed recombination activity, out of them, 4 colonies of each library were further analyzed for their activity on the loxF8 on-target (10 μg/ml L-arabinose) and on the Sym1 and Sym2 off-targets (200 μg/ml L-arabinose). All of the clones (L1-L12) showed recombination activity on the loxF8 site, but the efficiency varied between the clones (FIG. 17A). Two clones of library 1 and three clones of library 3 had activity on the Sym1 site at high induction level (FIG. 17B). Two clones of library 2 and all four clones of library 3 had measurable activity on the Sym2 site at high induction level (FIG. 17C). It is worth noting, that none of the clones from library 2 showed activity on the symmetric sites. Therefore, it can be assumed that the design of the library 2 was more beneficial to prevent activity on symmetric target sites. The picked clones were sequenced, and sequencing results demonstrated that the library strategy had worked (Table 4).

TABLE 4
Sequences of the single clones from the linker libraries

| Name | Amino acid sequence (5′→3′) | Nucleic acid sequence (5′→3′) |
|---|---|---|
| Ll (library 1) | AEATSEGGSGGSGGSGGSNGARRT | GCCGAAGCAACAAGCGAAGGCGGCAGC |
| | SEQ ID NO: 8 | GGTGGTAGCGGTGGCTCTGGCGGTAG |
| | | TAACGGCGCAAGAAGAACC |
| | | SEQ ID NO: 55 |
| L3 (library 1) | AGTTARGGSGGSGGSGGSGRRGAK | GCAGGAACAACCGCAAGAGGCGGCAGC |
| | SEQ ID NO: 9 | GGTGGTAGCGGTGGCTCTGGCGGTAG |
| | | TGGAAGAAGAGGAGCAAAA |
| | | SEQ ID NO: 56 |
| L4 (library 1) | KNGRGRGGSGGSGGSGGSRTKRET | AAAAACGGAAGAGGAAGAGGCGGCAG |
| | SEQ ID NO: 10 | CGGTGGTAGCGGTGGCTCTGGCGGTA |
| | | GTAGAACAAAAAGAGAAACA |
| | | SEQ ID NO: 57 |
| L5 (library 2) | GGSGGSTAAKEGAASSASGGSGGS | GGCGGTAGCGGTGGCTCTACCGCCGCC |
| | SEQ ID NO: 11 | AAAGAAGGAGCAGCAAGCAGCGCCAGC |
| | | GGTGGTAGTGGCGGCAGC |
| | | SEQ ID NO: 58 |
| L6 (library 2) | GGSGGSNSRSNTENSDKGGGSGGS | GGCGGTAGCGGTGGCTCTAACAGCAG |
| | SEQ ID NO: 12 | AAGCAACACAGAAAACAGCGACAAAGG |
| | | CGGTGGTAGTGGCGGCAGC |
| | | SEQ ID NO: 59 |
| L7 (library 2) | GGSGGSNGEEGTERGKATGGSGGS | GGCGGTAGCGGTGGCTCTAACGGCGA |
| | SEQ ID NO: 13 | AGAAGGAACCGAAAGAGGCAAAGCAAC |
| | | AGGTGGTAGTGGCGGCAGC |
| | | SEQ ID NO: 60 |
| L8 (library 2) | GGSGGSTTKANRAKGGRGGGSGGS | GGCGGTAGCGGTGGCTCTACCACCAA |
| | SEQ ID NO: 14 | AGCAAACAGAGCAAAAGGCGGCAGAGG |
| | | AGGTGGTAGTGGCGGCAGC |
| | | SEQ ID NO: 61 |
| L9 (library 3) | GANEDTNTEAAGSEGNEKTGTNSA | GGCGCAAACGAAGACACAAACACAGAA |
| | SEQ ID NO: 15 | GCAGCCGGAAGCGAAGGCAACGAAAAA |
| | | ACCGGAACAAACAGCGCT |
| | | SEQ ID NO: 62 |
| L11 (library 3) | GESRAEDGAKGNGRGKGEATAGAA | GGCGAAAGCAGAGCAGAAGACGGCGCA |
| | SEQ ID NO: 16 | AAAGGAAACGGCAGAGGCAAAGGAGAA |
| | | GCAACAGCAGGCGCAGCA |
| | | SEQ ID NO: 63 |

Based on the recombination efficiency on the loxF8 site, and the absence of the activity on the symmetric sites, clone L8 (S2-linker L8-S1) was chosen for further analyses.

### Example 6: Recombination Activity of Recombinases Fused with the L8-Linker

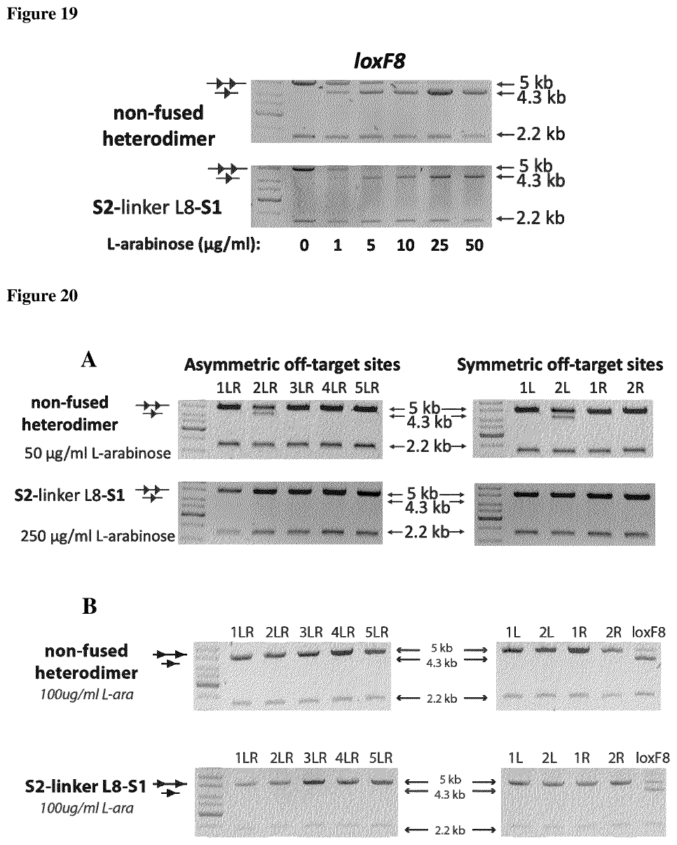

The recombinase-fusion with the L8-linker was tested on the loxF8 site at different induction levels in parallel with the (G₂S)₈ linker linker and the non-fused heterodimer. Test digest revealed that the L8-linker fusion recombined the plasmid more efficiently (FIG. 18). Likewise, the L8-linker fusion heterodimer also demonstrated higher recombination efficacy in comparison to the non-fused heterodimer (FIG. 19). Hence, fusion of the D7 monomers with the L8 linker sequence increases recombination activity.

### Example 7: Off-Target Activity of the Fused Heterodimer

A further goal was to find out whether fusion of the recombinase heterodimer increases the specificity of the recombination consequently minimizing potential off-target effects. Based on the remote similarity to the loxF8 sequence, human sequences that display high similarity to the loxF8 sequence, resembling potential asymmetric and symmetric off-target sites in the for the F8 recombinase heterodimer were tested in E. coli excision assays (Table 2, SEQ ID NOs: 19 to 29).

The activity on potential off-target sites was tested at high induction levels in order to identify even weak recombination activity. To account for possible different efficiencies of on-target recombination on the loxF8, the concentration of L-arabinose was choosen to be five times higher for the fused L8 heterodimer. Accordingly, L-arabinose concentrations of 50 μg/ml (for the non-fused) and 250 μg/ml (for the L8) were used for testing the heterodimer activity on five asymmetric and four symmetric off-targets.

For the D7 heterodimer, test digests revealed that at high induction levels the non-fused heterodimer showed marked recombination activity on one of the five tested asymmetric off-targets (2LR site, SEQ ID NO: 22 site). In contrast, the fused L8-heterodimer of the D7 recombinase did not show obvious recombination activity on any the asymmetric off-target sites (FIG. 20A).

On the symmetric off-target sites, the non-fused D7 heterodimer showed noticeable activity on one of the tested sites (2L site, SEQ ID NO: 27 site). The fused D7 heterodimer, on the contrary, did not display any activity on this, or any other of the tested sites (FIG. 20A). The results indicate that fusion of the heterodimer markedly increases recombination specificity of designer-SSRs.

For the A4 heterodimer, test digests using the non-fused heterodimer showed no marked recombination activity on the five tested asymmetric off-targets. The same holds true for the fused L8-heterodimer of the A4 recombinase (FIG. 20B).

On the symmetric off-target sites, the non-fused A4 heterodimer showed no activity on any of the tested sites. The fused A4 heterodimer did also not display any activity on this, or any other of the tested sites (FIG. 20B).

### Example 8: Recombination Activity in Human Cells

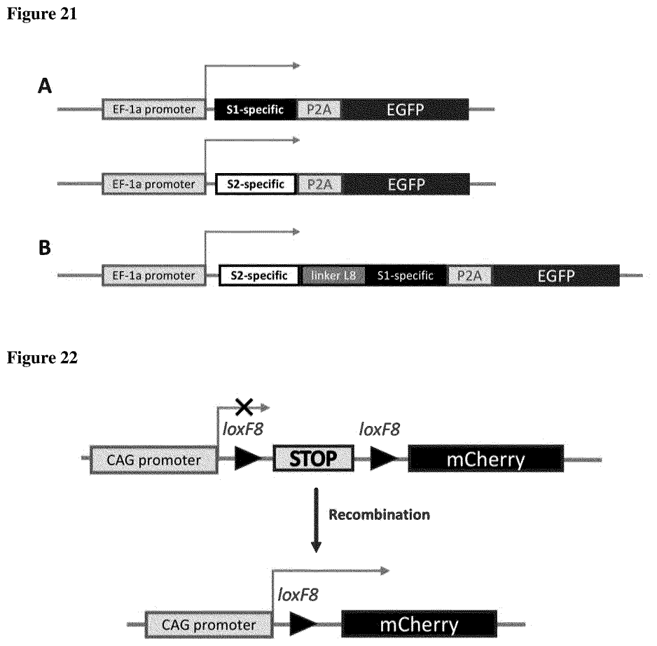

The fused L8-recombinase heterodimer has shown good activity in bacteria; therefore, the next step was to investigate the recombination efficacy in human cells and compare activities with the non-fused heterodimer. Transfection of HEK293T cells with an expression plasmid containing the fused or single recombinases transcriptionally fused with EGFP was performed (FIG. 21). The cells transfected with the empty plasmids expressing only EGFP and non-transfected cells were used as positive and negative control of the transfection, respectively.

Together with the recombinase expression plasmids, the cells were also transfected with a reporter plasmid, coding for mCherry with an upstream stop codon located between two loxF8 target sites. Upon recombination the reporter plasmid will excise the stop cassette, thereby allowing the expression of mCherry, which can be measured by flow cytometry analysis (FIG. 22).

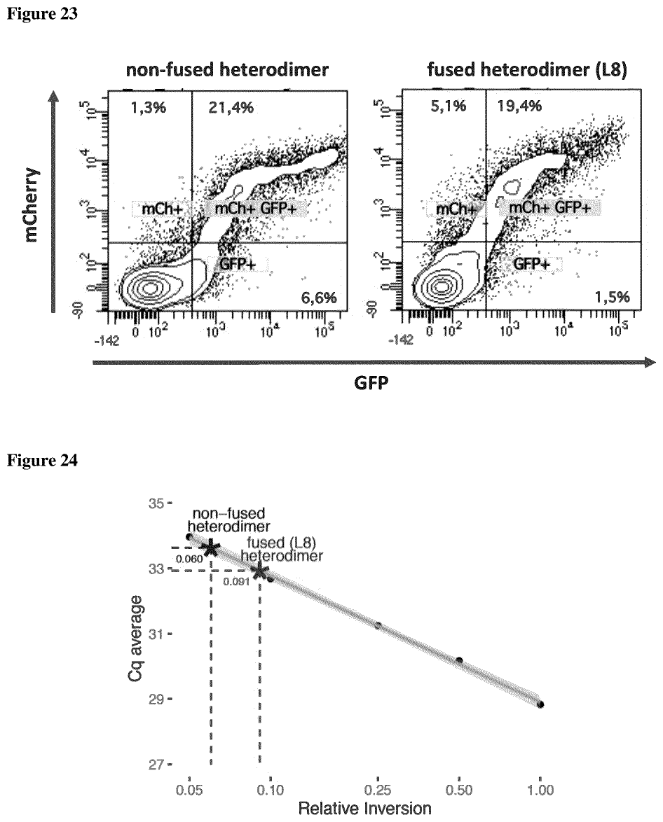

Flow cytometry analysis performed 48 h after transfection with the D7 recombinase heterodimer (comprising the recombinase monomers of SEQ ID NOs. 32 and 33; FIG. 23) revealed that the transfection efficiency of the cells transfected with both expression and reporter plasmids was 28% for the non-fused heterodimer, and 21% for the fused heterodimer, respectively. GFP and mCherry double positive cells reflect recombination of the reporter in human cells. In order to calculate the recombination efficiency of the reporter plasmid in human cells, the double positive cells were normalized to the transfection efficiency (FIG. 23). Accordingly, in 76.4% (non-fused) or 92.8% (fused) of the cells the reporters were recombined, indicating that the fused L8 recombinase has higher recombination efficiency in this assay.

### Example 9: Inversion Efficacy on Genomic Level in Human Cells

The here described recombinases were developed to correct a large gene inversion of exon 1 in the F8 gene which is causing Hemophilia A. To study the inversion efficacy of the heterodimers on genomic level, recombinases were expressed utilizing the described expression constructs (FIG. 21) for 48 h in HEK293T cells and gDNA was extracted subsequently. HEK293T cells do not carry the inverted exon 1 of the F8 gene. However, recombinases can carry out the inversion reaction independent on the orientation of the genomic DNA fragment between the loxF8 sites. Therefore, the ratio of the inverted to the non-inverted exon 1 can be used as a proxy for the inversion efficiency. A qPCR-based assay with TaqMan probes specific for the exon 1 inversion was developed to calculate the inversion frequency on genomic DNA. gDNA samples with defined amounts of exon 1 inversion were used to calculate a standard curve. The inversion efficiency of the recombinase transfected cells can be deduced afterwards using this standard curve. gDNA isolated from the samples transfected with the non-fused heterodimer showed that the inversion was induced in around 6.0% of cells. For samples transfected with the fused heterodimer the inversion frequency was around 9.0% (FIG. 24). However, since the transfection efficiency is not 100% the inversion rate should be corrected for GFP+ cells because GFP expression is transcriptionally linked to recombinase expression. Flow cytometry data revealed transfection efficacies of 29.5% (non-fused) and 21.3% (fused), respectively. Hence, the inversion efficacy on genomic level in recombinase expressing cells was 20.3% for the non-fused heterodimer and 42.7% for the fused L8 heterodimer. Thus, fusion of the L8 recombinase heterodimer resulted in two-fold higher inversion rate, demonstrating that fusion of recombinase heterodimers increases recombination activity.

### Example 10: Recombinase Activity on an Integrated Genomic loxF8 Reporter Construct

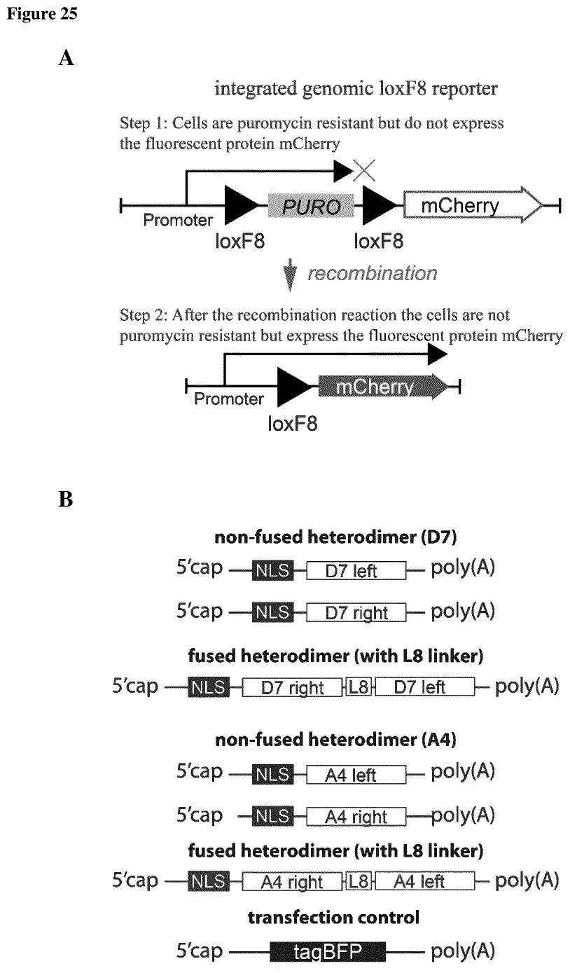

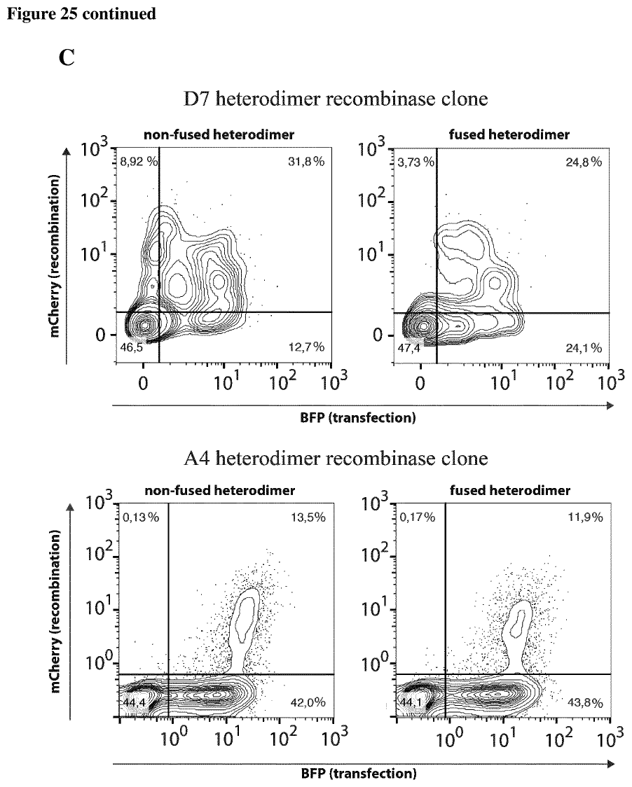

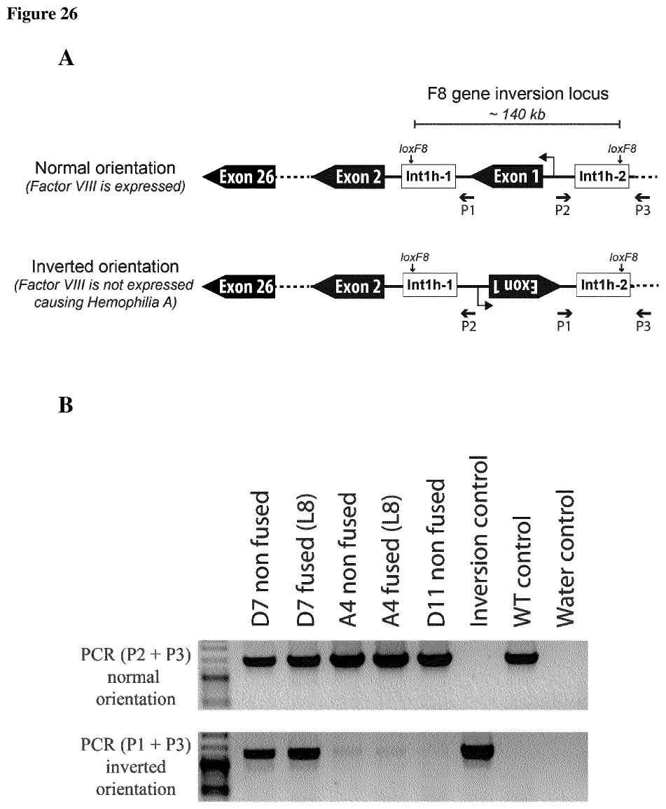

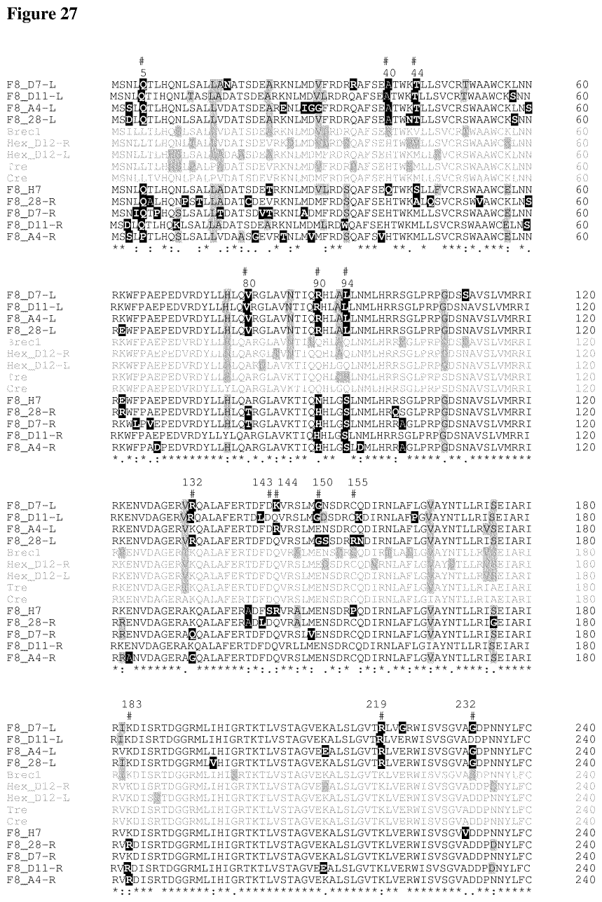

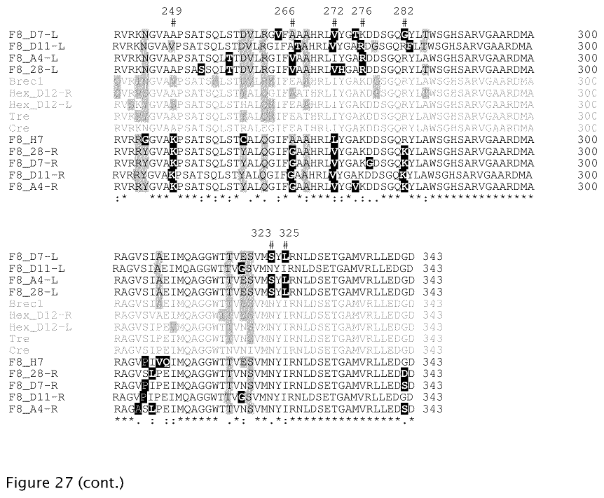

Since the recombinases were developed to recombine a sequence found in the human genome, a genomic loxF8 reporter cell line was developed (FIG. 25 A). Upon recombination of the loxF8 target site of the integrated reporter, the cells start expressing the red fluorescent protein mCherry. Recombinases were transfected to the reporter cell line as synthetic mRNAs (FIG. 25 B). A blue fluorescent protein (tagBFP) coding synthetic mRNA was cotransfected to estimate the transfection efficiency. Flow cytometry analysis performed 48 h after transfection with the D7 recombinase (non-fused and fused—comprising the recombinase monomers of SEQ ID NOs 32 and 33) and the A4 recombinase (non-fused and fused—comprising the recombinase monomers of SEQ ID NOs 93 and 94) together with the tagBFP mRNA revealed that the transfection efficiency was 44.5% for D7 non-fused, 48.9% for D7 fused, 55.5% for A4 non-fused and 55.7% for A4 fused. BFP and mCherry double positive cells reflect recombination of the genomic reporter in the human reporter cell line. In order to calculate the recombination efficiency of the genomic reporter, the double positive (BFP+ and mCherry+) cells were normalized to the transfection efficiency (FIG. 25 C). Accordingly, 71.5% for D7 non-fused, 50.7% for D7 fused, 24.3% for A4 non-fused and 21.4% for A4 fused of the genomic reporter were recombined.

### Example 11: Detection of the loxF8 Genomic Inversion of the Native loxF8 Locus Via PCR

HEK293T cells were transfected with synthetic mRNAs coding for the recombinase heterodimers D7 non-fused, D7 fused, A4 non-fused, A4 fused and D11 non-fused (comprising the recombinase monomers of SEQ ID NOs 32, 33, 93, 94, 99 and 100). Genomic DNA was extracted 48 h after transfection and analysed for the 140 kb inversion of the native loxF8 locus (FIG. 26 A). The orientation of the loxF8 locus can be detected by using different combinations of primers for a PCR (FIG. 26 A). Primers P2 and P3 were used to detect the normal orientation of the loxF8 locus and primers P1 and P3 were used to detect the inversion orientation of the loxF8 locus (FIG. 26 B). Only cells transfected with the synthetic mRNAs coding for the recombinase heterodimers show the inversion specific band (PCR P1+P3). No band is visible in the WT control or the water control sample. In order to confirm the integrity of the genomic DNA used in this experiment, the normal orientation PCR (P2+P3) was performed as well. All samples show a PCR band at the expected size indicating the genomic DNA used as a template for all the PCR reactions is intact. As expected the inversion control sample does not show a band in this reaction. These results indicate that the recombinase heterodimers of SEQ ID NOs.: 32 and 33 (non-fused D7 recombinase), SEQ ID NOs.: 93 and 94 (non-fused A4 recombinase) and SEQ ID NOs.: 99 and 100 (non-fused D11 recombinase) as well as the fusion proteins of SEQ ID NO: 72 (fused D7 recombinase) and of SEQ ID NO: 97 (fused A4 recombinase) can invert the 140 kb native loxF8 locus.

### REFERENCES

Abi-Ghanem, J., Chusainow, J., Karimova, M., Spiegel, C., Hofmann-Sieber, H., Hauber, J., Buchholz, F., Pisabarro, M. T. (2013). Engineering of a target site-specific recombinase by a combined evolution- and structure-guided approach. Nucleic Acids Research, 41: 2394-2403.
Albanese, C., Hulit, J., Sakamaki, T., Pestell, R. G. (2002). Recent advances in inducible expression in transgenic mice. Seminars in Cell and Developmental Biology, 13: 129-141.
Ausubel et al., Current Protocols in Molecular Biology (Supplement 47), John Wiley & Sons, New York (1999).
Buchholz, F., Stewart, A. F. (2001). Alteration of Cre recombinase site specificity by substrate-linked protein evolution. Nature Biotechnology, 19: 1047-1052.
Carcao, M. (2014). Changing paradigm of prophylaxis with longer acting factor concentrates. Haemophilia, 20: 99-105.
Carroll, D. (2014). Genome engineering with targetable nucleases. Annual Review of Biochemistry, 83: 409-439.
Castaldo, G., D'Argenio, V., Nardiello, P., Zarrilli, F., Sanna, V., Rocino, A., Coppola, A., Di Minno, G., Salvatore, F. (2007). Haemophilia A: molecular insights. Clinical chemistry and laboratory medicine, 45: 450-461.
Chen, X., Zaro, J. L., Shen, W. C. (2013). Fusion protein linkers: property, design and functionality. Advanced Drug Delivery Reviews, 65: 1357-1369.
Cox, D. B., Platt, R. J., Zhang, F. (2015). Therapeutic Genome Editing: Prospects and Challenges. Nature Medicine 21: 121-131.
Cyranoski, D. (2016). CRISPR gene-editing tested in a person for the first time. Nature, 539: 479.
Dahlback, B. (2000). Blood coagulation. The Lancet, 355: 1627-1632.
Eroshenko, N., Church, G. M. (2013). Mutants of Cre recombinase with improved accuracy. Nature Communications, 4: 2509.
Evan, G. I., Lewis, G. K., Ramsay, G., Bishop, J. M. (1985). Molecular and Cell Biology, 5: 3610-3616.
Fischer, K., Bom, J. G., Mauser-Bunschoten, E. P., Roosendaal, G., Berg, H. M. (2005). Effects of haemophilic arthropathy on health-related quality of life and socio-economic parameters. Haemophilia, 11: 43-48.
Gelato, K. A., Martin, S. S., Liu, P. H., Saunders, A. A., Baldwin, E. P. (2008). Spatially-Directed Assembly of a Heterotetrameric Cre-Lox Synapse Restricts Recombination Specificity. Journal of Molecular Biology, 378: 653-665.
George, L. A., Ragni, M. V., Samelson-Jones, B. J., Cuker, A., Runoski, A. R., Cole, G., Wright, F., Chen, Y., Hui, D. J, Wachtel, K., Takefman, D., Couto, L. B., Reape, KZ, Carr, M. E., Anguela, X. M., High, K. A. (2017). Spk-8011: preliminary results from a phase ½ dose escalation trial of an investigational AAV-mediated gene therapy for hemophilia A. Blood, 130: 604.
Gouw, S., van der Born, J. G., Ljung, R., Escuriola, C., Cid, A. R., Claeyssens-Donadel, S., van Geet, C., Kenet, G., Makipernaa, A., Molinari, A. C., Muntean, W., Kobelt, R., Rivard, G., Santagostino, E., Thomas, A., van den Berg, H. M., PedNet and RODIN Study Group. (2013). Factor VIII Products and Inhibitor Development in Severe Hemophilia A. The New England Journal of Medicine, 368: 231-239.
Graw, J., Brackmann, H. H., Oldenburg, J., Schneppenheim, R., Spannagl, M., Schwaab, R. (2005). Haemophilia A: from mutation analysis to new therapies. Nature Reviews Genetics, 6: 488-501.
Hauber, I., Hofmann-Sieber, H., Chemnitz, J., Dubrau, D., Chusainow, J., Stucka, R., Hartjen, P., Schambach, A., Ziegler, P., Hackmann, K., Schrock, E., Schumacher, U., Lindner, C., Grundhoff, A., Baum, C., Manz, M. G., Buchholz, F., Hauber, J. (2013). Highly significant antiviral activity of HIV-1 LTR-specific tre-recombinase in humanized mice. PLoS Pathogens, 9: e1003587.
Justice, M. J., Siracusa, L. D., Stewart, A. F. (2011). Technical approaches for mouse models of human disease. Disease Model and Mechanisms, 4: 305-310.
Karimova, M., Splith, V., Karpinski, J., Pisabarro, M. T., Buchholz, F. (2016). Discovery of Nigri/nox and Panto/pox site-specific recombinase systems facilitates advanced genome engineering. Scientific Reports, 6: 30130.
Karpinski, J., Hauber, I., Chemnitz, J., Schafer, C., Paszkowski-Rogacz, M., Chakraborty, D., Beschorner, N., Hofmann-Sieber, H., Lange, U. C., Grundhoff, A., Hackmann, K., Schrock, E., Abi-Ghanem, J., Pisabarro, M. T., Surendranath, V., Schambach, A., Lindner, C., van Lunzen, J., Hauber, J., Buchholz, F. (2016). Directed evolution of a recombinase that excises the provirus of most HIV-1 primary isolates with high specificity. Nature Biotechnology, 34: 401-409.
Konkle, B., Stasyshyn, O., Chowdary, P., Bevan, D. H., Mant, T., Shima, M., Engl, W., Dyck-Jones, J., Fuerlinger, M., Patrone, L., Ewenstein, B., Abbuehl, B. (2015). Pegylated, full-length, recombinant factor VIII for prophylactic and on-demand treatment of severe hemophilia A. Blood, 126: 1078-1085.
Kosicki, M., Tomberg, K., Bradley, A. (2018). Repair of double-strand breaks induced by CRISPR-Cas9 leads to large deletions and complex rearrangements. Nature Biotechnology, 36: 765-771.
Lansing, F., Paszkowski-Rogacz, M., Schmitt, L. T., Schneider M. P., Rojo Romanos, T., Sonntag, J., Buchholz, F. (2020). A heterodimer of evolved designer-recombinases precisely excises a human genomic DNA locus. Nucleic Acids Research. 48(1):472-485. doi: 10.1093/nar/gkz1078
Lee, H. J., Kweon, J., Kim, E., Kim, S., Kim, J. S. (2012). Targeted chromosomal duplications and inversions in the human genome using zinc finger nucleases. Genome Research, 22: 539-548.
Lenting, P., Denis, C. V., Christophe, O. D. (2017). Emicizumab, a bispecific antibody recognizing coagulation factors IX and X: how does it actually compare to factor VIII?Blood, 130: 2463-2468.
Mahlangu, J., Powell, J. S., Ragni, M. V., Chowdary, P., Josephson, N.C., Pabinger, I., Hanabusa, H., Gupta, N., Kulkarni, R., Fogarty, P., Perry, D., Shapiro, A., Pasi, K. J., Apte, S., Nestorov, I., Jiang, H., Li, S., Neelakantan, S., Cristiano, L. M., Goyal, J., Sommer, J. M., Dumont, J. A., Dodd, N., Nugent, K., Vigliani, G., Luk, A., Brennan, A., Pierce, G. F., A-LONG Investigators. (2014). Phase 3 study of recombinant factor VIII Fc fusion protein in severe hemophilia A. Blood, 123: 317-325.
Meinke, G., Bohm, A., Hauber, J., Pisabarro, M. T., Buchholz, F. (2016). Cre recombinase and other tyrosine recombinases. Chemical reviews, 116: 12785-12820.
Melchiorre, D., Manetti, M., Matucci-Cerinic, M. (2017). Pathophysiology of Hemophilic Arthropathy. Journal of clinical medicine, 6: 63.
O'Hara, J., Hughes, D., Camp, C., Burke, T., Carroll, L., Diego, D. G. (2017). The cost of severe haemophilia in Europe: the CHESS study. Orphanet journal of rare diseases, 12: 106.
Pandey G., Mittal B. (2001). Molecular diagnosis in haemophilia A. Journal of Postgraduate Medicine, 47: 274.
Park, C. Y., Kim, J., Kweon, J., Son, J. S., Lee, J. S., Yoo, J. E., Cho, S. R., Kim, J. H., Kim, J. S., Kim, D. W. (2014). Targeted inversion and reversion of the blood coagulation factor 8 gene in human iPS cells using TALENs. roceedings of the National Academy of Sciences USA, 111: 9253-9258.
Park, C. Y., Kim, D. H., Son, J. S., Sung, J. J., Lee, J., Bae, S., Kim, J. H., Kim, D. W., Kim, J. S. (2015). Functional Correction of Large Factor VIII Gene Chromosomal Inversions in Hemophilia A Patient-Derived iPSCs Using CRISPR-Cas9. Cell Stem Cell, 17: 213-220.
Pasi, K. J., Rangarajan, S., Kim, B., Lester, W., Perry, D., Madan, B., Tavakkoli, F., Yang, K., Pierce, G. F., Wong, W. Y. (2017). Achievement of normal circulating factor VIII activity following Bmn 270 AAV5-FVIII gene transfer: interim, long-term efficacy and safety results from a phase ½ study in patients with severe hemophilia A. Blood, 130: 603.
Peters, R., Harris, T. (2018). Advances and innovations in haemophilia treatment. Nature Review Drug Discovery, 17: 493-508.
Peyvandi F., Garagiola, I., Young, G. (2016). The past and future of haemophilia: diagnosis, treatments, and its complications. Lancet, 388: 187-97.
Qasim, W., Amrolia, P. J., Samarasinghe, S., Ghorashian, S., Zhan, H., Stafford, S., Butler, K., Ahsan, G., Gilmour, K., Adams, S., Pinner, D., Chiesa, R., Chatters, S., Swift, S., Goulden, N., Peggs, K., Thrasher, A. J., Veys, P., & Pule, M. (2015). First Clinical Application of Talen Engineered Universal CAR19 T Cells in B-ALL. Blood, 126: 2046.
Santoro, S. W., Schultz, P. G. (2002). Directed evolution of the site specificity of Cre recombinase. Proceedings of the National Academy of Sciences, 99: 4185-4190.
Saraf-Levy, T., Santoro, S. W., Volpin, H., Kushnirsky, T., Eyal, Y., Schultz, P. G., Gidoni, D., Carmi, N. (2006). Site-specific recombination of asymmetric lox sites mediated by a heterotetrameric Cre recombinase complex. Bioorganic & Medicinal Chemistry, 14: 3081-3089.
Sarkar, I., Hauber, I., Hauber, J., Buchholz, F. (2007). HIV-1 proviral DNA excision using an evolved recombinase. Science, 316, 1912-1915.
Shahani, T., Covens, K., Lavend'homme, R., Jazouli, N., Sokal, E., Peerlinck, K., Jacquemin, M. (2014). Human liver sinusoidal endothelial cells but not hepatocytes contain factor VIII. Journal of Thrombosis and Haemostasis, 12: 36-42.
Tebas, P., Stein, D., Tang, W., Frank, I., Wang, S. Q., Lee, G., Spratt, S. K., Surosky, R. T., Giedlin, M. A., Nichol, G., Holmes, M. C., Gregory, P. D., Ando, D. G., Kalos, M., Collman, R. G., Binder-Scholl, G., Plesa, G., Hwang, W. T., Levine, B. L., June, C. H. (2014). Gene editing of CCR5 in autologous CD4 T cells of persons infected with HIV. The New England Journal of Medicine, 370: 901-910.
Tizzano E. F., Cornet, M., Baiget, M. (2003). Inversion of intron 1 of the factor VIII gene for direct molecular diagnosis hemophilia A. Haematologica, 88:118-20.
Turner, N., Moake, J. (2000). Factor VIII Is Synthesized in Human Endothelial Cells, Packaged in Weibel-Palade Bodies and Secreted Bound to ULVWF Strings. PLoS One, 10.
Vandamme, C., Adjali, O., Mingozzi, F. (2017). Unraveling the complex story of immune responses to AAV vectors trial after trial. Human Gene Therapy, 28: 1061-1074.
Wang, M., Glass, Z. A., Xu, Q. (2017). Non-viral delivery of genome-editing nucleases for gene therapy. Gene Therapy, 24: 144-150.
White, G., Rosendaal, F., Aledort, L. M., Lusher, J. M., Rothschild, C., Ingerslev, J., Factor VIII and Factor IX Subcommittee. (2001). Recommendation of the scientific subcommittee on factor VIII and factor IX of the scientific and standardization committee of the International Society on Thrombosis and Haemostasis. Thrombosis and Haemostasis, 85: 560.
Wilson, I. A., Niman, H. L., Houghten, R. A., Cherenson, A. R., Connolly, M. L., Lerner, R. A. (1984). The structure of an antigenic determinant in a protein. Cell, 37: 767-778.
Yu, Y., Bradley, A. (2002). Engineering chromosomal rearrangements in mice. Nature Review Genetics, 2: 780-790.
Zhang, C., Myers, C. A., Qi, Z., Mitra, R. D., Corbo, J. C., Havranek, J. J. (2015). Redesign of the monomer-monomer interface of Cre recombinase yields an obligate heterotetrameric complex. Nucleic Acids Research, 43: 9076-9085.

1. A method for generating a fusion protein of DNA recombinases for genome editing, preferably for recombination, more preferably for inversion of DNA sequences on genomic level in a cell, wherein said method comprises the steps of:
i. identifying a nucleic acid sequence that is a potential target site for DNA-recombining enzymes that are capable to induce a site-specific DNA recombination of a sequence of interest in a genome, wherein said potential target site comprises two asymmetric recombinase target sites;
ii. providing a nucleic acid molecule encoding a first recombinase enzyme, wherein said first recombinase enzyme has been evolved by directed evolution or rational design to specifically recognize a first half-site of a recombinase target site;
iii. providing a nucleic acid molecule encoding a second recombinase enzyme, wherein said second recombinase enzyme has been evolved by directed evolution or rational design to specifically recognize a second half-site of a recombinase target site;
iv. providing a nucleic acid molecule encoding a linker peptide, wherein said nucleic acid encodes a linker oligopeptide comprising 6 to 30 amino acids;
v. creating an expression vector by cloning the nucleic acid molecule encoding a first recombinase enzyme, the nucleic acid molecule encoding a second recombinase enzyme and the nucleic acid molecule encoding a linker peptide into an expression vector;
vi. transfecting a cell, which comprises a DNA sequence, which is to be recombined, preferably inverted, with the expression vector of step v);
vii. expressing the fusion protein comprising a first recombinase enzyme, a second recombinase enzyme and a linker peptide;
viii. analyzing, whether the fusion protein expressed in step vii. is capable of inverting the DNA sequence on a human chromosome in said cell; and
ix. selecting a fusion protein which is capable of inverting of DNA sequence on a human chromosome in said cell according to step viii.

2. The method of claim 1, further comprising the steps of:
x. Identifying potential off-target sites of the desired fusion protein;
xi. Analyzing the recombinase activity of the desired fusion protein on the off-target sites identified in step x.; and
xii. Selecting fusion proteins that do not show recombinase activity on at least one off-target site.

3. The method of claim 1, wherein in the fusion protein expressed in step vii., the C-terminus of the said first recombinase enzyme is connected to the N-terminus of said second recombinase enzyme via the linker peptide; or wherein the C-terminus of the said second recombinase enzyme is connected to the N-terminus of said first recombinase enzyme via the linker peptide.

4. The method according to claim 1, wherein step i. of the method of claim 1 comprises the substeps of:
a) Screening the genome or a part thereof comprising the sequence of interest for two sequences that are potential spacer sequences, with a length of at least 5 and up to 12 bp, wherein one of the potential spacer sequence lies upstream of the sequence of interest and the other potential spacer sequence lies downstream of the sequence of interest and wherein the two sequences have a maximum distance of 2 megabases and a minimum distance of 150 bp,
b) Identifying potential target sites by determining for each potential spacer sequence the neighboring nucleotides, preferably 10 to 20 nucleotides, more preferably 12 to 15 nucleotides, most preferably 13 nucleotides on one side thereof form the potential first half site and the neighboring nucleotides, preferably 10 to 20 nucleotides, more preferably 12 to 15 nucleotides, most preferably 13 nucleotides, on the other side form the potential second half site, whereas both potential half sites and the spacer sequence in between form a potential target site,
c) The potential target sites identified in step b) are further screened to select for potential target sequences that do not occur (elsewhere) in the genome of the host to ensure a sequence specific recombination, preferably inversion.

5. The method according to claim 1, wherein first recombinase enzyme and the second recombinase enzyme according to steps ii. and iii. are evolved by substrate linked directed evolution (SLiDE), wherein said substrate linked directed evolution comprises the steps of:
a) Selecting a nucleotide sequence upstream of the nucleotide sequence to be altered as first target site and a nucleotide sequence downstream of the nucleotide sequence to be altered as second target site, whereas the sequences of the target sites are preferably not identical, wherein each target site comprises a first half site and a second half site with each 10 to 20 nucleotides separated by a spacer sequence with 5 to 12 nucleotides,
b) Applying molecular directed evolution on at least one library of DNA-recombining enzymes using a vector comprising the first target site and the second target site as selected in a) as substrate,
until at least one first designer DNA-recombining enzyme is obtained that is active on the first target site and at least one second designer DNA-recombining enzyme is obtained that is active on the second target site as selected in a).

6. A fusion protein for genome editing, which has been obtained by the method according to claim 1, wherein said fusion protein consists of a complex of recombinases, wherein said complex consists of at least a first recombinase enzyme, a second recombinase enzyme and at least one linker, wherein said first recombinase enzyme and said second recombinase enzyme specifically recognize a first half-site and a second half-site of an upstream target site and/or a downstream target site of a recombinase; wherein said first recombinase enzyme and said second recombinase enzyme are interconnected via a linker; and wherein said linker comprises or consists of an oligopeptide comprising 4 to 50 amino acids; wherein said fusion protein specifically recognizes the upstream recombinase target sequence of the loxF8 target site, which has the nucleic acid sequence
ATAAATCTGTGGAAACGCTGCCACACAATCTTAG (SEQ ID NO: 17) or a reverse complement sequence thereof;
and recognizes the downstream recombinase target sequence of the loxF8 target site, which has the nucleic acid sequence
(SEQ ID NO: 18)
CTAAGATTGTGTGGCAGCGTTTCCACAGATTTAT
or a reverse complement sequence thereof;
and which catalyzes the inversion of a gene sequence between the upstream recombinase target sequence of SEQ ID NO. 17 and the downstream recombinase target sequence of SEQ ID NO: 18 of the loxF8 recombinase target site; and
wherein the capability to catalyzes the inversion of a gene sequence between the upstream recombinase target sequence of SEQ ID NO. 17 and the downstream recombinase target sequence of SEQ ID NO: 18 of the loxF8 recombinase target site is tested by a method comprising the steps of:
a) expressing the fusion protein comprising a first recombinase enzyme, a second recombinase enzyme and a linker peptide; and
b) analyzing, whether the fusion protein expressed in step a) is capable of inverting of DNA sequence on a human chromosome in said cell.

7. The fusion protein of claim 6, wherein said linker
comprises or consists of an oligopeptide with an amino acid sequence selected from formula 1, formula 2 and formula 3:
X₁-X₂-X₃-X₄-X₅-X₆-(G₂S)₄-X₇-X₈-X₉-X₁₀-X₁₁-X₁₂ (formula 1);
(G₂S)₂-X₁-X₂-X₃-X₄-X₅-X₆-X₇-X₈-X₉-X₁₀-X₁₁-X₁₂-(G₂S)₂ (formula 2); and
X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄ (formula 3);
wherein
G is glycine;
S is serine; and
each of X₁ to X₂₄ is independently selected from the group consisting of alanine, arginine, asparagine, aspartic acid, glutamine, glycine, lysine, serine and threonine;
wherein said oligopeptide of formula 1, formula 2 or formula 3 does not consist of glycine and serine residues only.
or comprises or consists of an oligopeptide selected from the group consisting of
(SEQ ID NO: 8)
AEATSEGGSGGSGGSGGSNGARRT;
(SEQ ID NO: 9)
AGTTARGGSGGSGGSGGSGRRGAK;
(SEQ ID NO: 10)
KNGRGRGGSGGSGGSGGSRTKRET;
(SEQ ID NO: 11)
GGSGGSTAAKEGAASSASGGSGGS;
(SEQ ID NO: 12)
GGSGGSNSRSNTENSDKGGGSGGS;
(SEQ ID NO: 13)
GGSGGSNGEEGTERGKATGGSGGS;
(SEQ ID NO: 14)
GGSGGSTTKANRAKGGRGGGSGGS;
(SEQ ID NO: 15)
GANEDTNTEAAGSEGNEKTGTNSA;
and
(SEQ ID NO: 16)
GESRAEDGAKGNGRGKGEATAGAA.

8. The fusion protein according to claim 6, wherein in said fusion protein, the first recombinase enzyme, the second recombinase enzyme and the linker are interconnected such that the C-terminus of the second recombinase enzyme, which specifically recognizes a second half-site of a recombinase target site, is fused with the linker to the N-terminus of the first recombinase enzyme, which specifically recognizes a first half-site of a recombinase target site.;
or wherein in said fusion protein, the first recombinase enzyme, the second recombinase enzyme and the linker are interconnected such that the C-terminus of the first recombinase enzyme, which specifically recognizes a first half-site of a recombinase target site, is fused with the linker to the N-terminus of the second recombinase enzyme, which specifically recognizes a second half-site of a recombinase target site

9. The fusion protein according to claim 6, wherein each recombinase enzyme of said heterodimer is a tyrosine site-specific recombinase, which has been evolved by directed evolution to independently recognize a first half-site and a second-half site of a recombinase target site of tyrosine site-specific recombinases, optionally wherein said recombinase target site is a target site of a tyrosine site-specific recombinase is selected from the group consisting of Cre-, Dre-, VCre-, SCre-, Vika-, lambda-Int-, Flp-, R-, Kw-, Kd-, B2-, B3-, Nigri- and Panto-recombinase.

10. (canceled)

11. The fusion protein according to claim 6, wherein said first recombinase enzyme is a protein, which has an amino acid sequence with at least 70%, preferably 80%, more preferably 90%, sequence identity with a sequence according to SEQ ID NO: 30, SEQ ID NO: 32, SEQ ID NO: 93 or SEQ ID NO: 99 and/or wherein said second recombinase enzyme is a protein, which has an amino acid sequence with at least 70%, preferably 80%, more preferably 90%, sequence identity with a sequence according to SEQ ID NO: 31, SEQ ID NO: 33, SEQ ID NO: 94 or SEQ ID NO: 100;
or wherein said fusion protein has an amino acid sequence with at least 70%, preferably 80%, more preferably 90%, sequence identity with a sequence according to SEQ ID NO: 71, SEQ ID NO: 72, SEQ ID NO: 97 or SEQ ID NO: 103, wherein SEQ ID NO: 71, SEQ ID NO: 72, SEQ ID NO: 97 and SEQ ID NO: 103 each include a linker with the amino acid sequence of SEQ ID NO: 14.

12. A fusion protein consisting of a first recombinase enzyme and a second recombinase enzyme, wherein said first recombinase enzyme and said second recombinase enzyme specifically recognize a first half-site and a second half-site of an upstream target site and/or a downstream target site of a recombinase, wherein said first recombinase enzyme is a polypetide, which has an amino acid sequence with at least 70%, preferably 80%, more preferably 90%, sequence identity with a sequence according to SEQ ID NO: 30, SEQ ID NO: 32, SEQ ID NO: 93 or SEQ ID NO: 99 and/or wherein said second recombinase enzyme is a polypeptide, which has an amino acid sequence with at least 70%, preferably 80%, more preferably 90%, sequence identity with a sequence according to SEQ ID NO: 31, SEQ ID NO: 33, SEQ ID NO: 94 or SEQ ID NO: 100; wherein DNA recombinase heterodimer specifically recognizes the upstream recombinase target sequence of the loxF8 target site, which has the nucleic acid sequence
ATAAATCTGTGGAAACGCTGCCACACAATCTTAG (SEQ ID NO: 17), or a reverse complement sequence thereof;
and recognizes the downstream recombinase target sequence of the loxF8 target site, which has the nucleic acid sequence
CTAAGATTGTGTGGCAGCGTTTCCACAGATTTAT (SEQ ID NO: 18), or a reverse complement sequence thereof;
and which catalyzes the inversion of a gene sequence between the upstream recombinase target sequence of SEQ ID NO. 17 and the downstream recombinase target sequence of SEQ ID NO: 18 of the loxF8 recombinase target site;
wherein the capability to catalyzes the inversion of a gene sequence between the upstream recombinase target sequence of SEQ ID NO. 17 and the downstream recombinase target sequence of SEQ ID NO: 18 of the loxF8 recombinase target site is tested by a method comprising the steps of:
a) expressing the fusion protein comprising a first recombinase enzyme, a second recombinase enzyme and a linker peptide;
b) analyzing, whether the fusion protein expressed in step a) is capable of inverting of DNA sequence on a human chromosome in said cell.

13. A nucleic acid molecule encoding a fusion protein according to claim 6.

14. The nucleic acid molecule of claim 13, which comprises or consists of
i. nucleic acids with the sequence of SEQ ID NOs 34, 35 and 61; such as the nucleic acid of SEQ ID NO 64; or
ii. nucleic acids with the sequence of SEQ ID NOs 36, 37 and 61; such as the nucleic acid of SEQ ID NO 65; or
iii. nucleic acids with the sequence of SEQ ID NOs 95, 96 and 61; such as the nucleic acid of SEQ ID NO 98; or
iv. nucleic acids with the sequence of SEQ ID NOs 101, 102 and 61; such as the nucleic acid of SEQ ID NO 104; or
v. nucleic acids with the sequence of SEQ ID NOs 95 and 96; or
vi. nucleic acids with the sequence of SEQ ID NOs 101 and 102;
vii. nucleic acids with the sequence of SEQ ID NOs 34 and 35; or
viii. nucleic acids with the sequence of SEQ ID NOs 36 and 37.

15. A polynucleotide molecule comprising the nucleic acid molecule according to claim 13 plus expression-controlling elements operably linked with said nucleic acid to drive expression thereof.

16. A mammalian, insect, plant or bacterial host cell comprising a nucleic acid molecule according to claim 13.

17. A pharmaceutical composition comprising a fusion protein according to claim 6 optionally in combination with one or more therapeutically acceptable diluents or carriers.

18. A method of treating hemophilia A comprising administering to a subject with hemophilia A the fusion protein according to claim 6.

19. A method for inversion of a DNA sequence on genomic level in a cell in vitro, comprising a fusion protein for genome editing according to the invention, wherein said method comprises the steps of:
i. providing a nucleic acid molecule encoding a first recombinase enzyme, wherein said first recombinase enzyme has been evolved by directed evolution or rational design to specifically recognize a first half-site of a recombinase target site;
ii. providing a nucleic acid molecule encoding a second recombinase enzyme, wherein said second recombinase enzyme has been evolved by directed evolution or rational design to specifically recognize a second half-site of a recombinase target site;
iii. providing a nucleic acid molecule encoding a linker peptide, wherein said nucleic acid encodes a linker oligopeptide comprising 6 to 30 amino acids;
iv. creating an expression vector by cloning the nucleic acid molecule encoding a first recombinase enzyme, the nucleic acid molecule encoding a second recombinase enzyme and the nucleic acid molecule encoding a linker peptide into an expression vector;
v. delivering to a cell, which comprises a DNA sequence, which is to be inverted, an expression vector of step iv), a RNA molecule encoding a fusion protein comprising at least a first recombinase enzyme, a second recombinase enzyme and at least one linker or a fusion protein comprising at least a first recombinase enzyme, a second recombinase enzyme and at least one linker;
vi. inversion of the DNA sequence on a human chromosome in said cell.

20. The method of claim 19, wherein said method further comprises the step
v. a) expressing the fusion protein comprising a first recombinase enzyme, a second recombinase enzyme and a linker peptide.

21. A loxF8 recombinase target site comprising a 5′ target sequence of SEQ ID NO: 17 and a 3′ target sequence of SEQ ID NO: 18 or a reverse complement sequence thereof.

22. A fusion protein comprising at least a first and a second recombinase attached via a linker, wherein the first and second recombinase are engineered from the same naturally occurring source recombinase, wherein said first recombinase specifically recognizes a first genomic nucleic acid sequence target site of the recombinase and said second recombinase specifically recognizes a second genomic nucleic acid sequence target site of the recombinase.

23. The fusion protein of claim 22, wherein the linker comprises an oligopeptide comprising 4 to 50 amino acids.

24. The fusion protein of claim 22, wherein the linker comprises or consists of an oligopeptide with an amino acid sequence selected from formula 1, formula 2 and formula 3:
X₁-X₂-X₃-X₄-X₅-X₆-(G₂S)₄-X₇-X₈-X₉-X₁₀-X₁₁-X₁₂ (formula 1);
(G₂S)₂-X₁-X₂-X₃-X₄-X₅-X₆-X₇-X₈-X₉-X₁₀-X₁₁-X₁₂-(G₂S)₂ (formula 2); and
X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄ (formula 3);
wherein
G is glycine;
S is serine; and
each of X₁ to X₂₄ is independently selected from the group consisting of alanine, arginine, asparagine, aspartic acid, glutamine, glycine, lysine, serine and threonine;
wherein said oligopeptide of formula 1, formula 2 or formula 3 does not consist of glycine and serine residues only.

25. The fusion protein of claim 22, wherein the linker comprises or consists of an oligopeptide selected from the group consisting of
(SEQ ID NO: 8)
AEATSEGGSGGSGGSGGSNGARRT;
(SEQ ID NO: 9)
AGTTARGGSGGSGGSGGSGRRGAK;
(SEQ ID NO: 10)
KNGRGRGGSGGSGGSGGSRTKRET;
(SEQ ID NO: 11)
GGSGGSTAAKEGAASSASGGSGGS;
(SEQ ID NO: 12)
GGSGGSNSRSNTENSDKGGGSGGS;
(SEQ ID NO: 13)
GGSGGSNGEEGTERGKATGGSGGS;
(SEQ ID NO: 14)
GGSGGSTTKANRAKGGRGGGSGGS;
(SEQ ID NO: 15)
GANEDTNTEAAGSEGNEKTGTNSA;
and
(SEQ ID NO: 16)
GESRAEDGAKGNGRGKGEATAGAA.


SEQUENCE LISTING (106 sequences)

SEQ ID NO: 1 - protein, 6 aa, Artificial sequence
  Synthetic peptide
GGSGGS

SEQ ID NO: 2 - protein, 12 aa, Artificial sequence
  Synthetic peptide
GGSGGSGGSGGS

SEQ ID NO: 3 - protein, 18 aa, Artificial sequence
  Synthetic peptide
GGSGGSGGSGGSGGSGGS

SEQ ID NO: 4 - protein, 24 aa, Artificial sequence
  Synthetic peptide
GGSGGSGGSGGSGGSGGSGGSGGS

SEQ ID NO: 5 - protein, 30 aa, Artificial Sequence
  Synthetic peptide
GGSGGSGGSGGSGGSGGSGGSGGSGGSGGS

SEQ ID NO: 6 - protein, 36 aa, Artificial Sequence
  Synthetic peptide
GGSGGSGGSGGSGGSGGSGGSGGSGGSGGSGGSGGS

SEQ ID NO: 7 - protein, 42 aa, Artificial Sequence
  Synthetic peptide
GGSGGSGGSGGSGGSGGSGGSGGSGGSGGSGGSGGSGGSGGS

SEQ ID NO: 8 - protein, 24 aa, Artificial Sequence
  Synthetic peptide
AEATSEGGSGGSGGSGGSNGARRT

SEQ ID NO: 9 - protein, 24 aa, Artificial Sequence
  Snthetic peptide
AGTTARGGSGGSGGSGGSGRRGAK

SEQ ID NO: 10 - protein, 24 aa, Artificial Sequence
  Synthetic peptide
KNGRGRGGSGGSGGSGGSRTKRET

SEQ ID NO: 11 - protein, 24 aa, Artificial Sequence
  Synthetic peptide
GGSGGSTAAKEGAASSASGGSGGS

SEQ ID NO: 12 - protein, 24 aa, Artificial Sequence
  Synthetic peptide
GGSGGSNSRSNTENSDKGGGSGGS

SEQ ID NO: 13 - protein, 24 aa, Artificial Sequence
  Synthetic peptide
GGSGGSNGEEGTERGKATGGSGGS

SEQ ID NO: 14 - protein, 24 aa, Artificial Sequence
  Synthetic peptide
GGSGGSTTKANRAKGGRGGGSGGS

SEQ ID NO: 15 - protein, 24 aa, Artificial Sequence
  Synthetic peptide
GANEDTNTEAAGSEGNEKTGTNSA

SEQ ID NO: 16 - protein, 24 aa, Artificial Sequence
  Synthetic peptide
GESRAEDGAKGNGRGKGEATAGAA

SEQ ID NO: 17 - DNA, 34 bp, Homo sapiens
ataaatctgtggaaacgctgccacacaatcttag

SEQ ID NO: 18 - DNA, 34 bp, Homo sapiens
ctaagattgtgtggcagcgtttccacagatttat

SEQ ID NO: 19 - DNA, 34 bp, Homo sapiens
ataaatctgtggaaacgctgctccacagatttat

SEQ ID NO: 20 - DNA, 34 bp, Homo sapiens
ctaagattgtgtgaacgctgccacacaatcttag

SEQ ID NO: 21 - DNA, 34 bp, Homo sapiens
ataaatttgtggaaattaaacaacacactcttaa

SEQ ID NO: 22 - DNA, 34 bp, Homo sapiens
acaaaattgtggaaattaaacaacacaatcttaa

SEQ ID NO: 23 - DNA, 34 bp, Homo sapiens
acaaatatgtggaaattaaacaacacactcttga

SEQ ID NO: 24 - DNA, 34 bp, Homo sapiens; identical to SEQ ID NO: 21

SEQ ID NO: 25 - DNA, 34 bp, Homo sapiens
ttaagagtgtgttttttaatttccacatatttgt

SEQ ID NO: 26 - DNA, 34 bp, Homo sapiens
ataaatatgtgaatatacatattcacatatttat

SEQ ID NO: 27 - DNA, 34 bp, Homo sapiens
atatatctatagatatagatatccacagatatat

SEQ ID NO: 28 - DNA, 34 bp, Homo sapiens
ctatgtttttgaggtcttattcacaaaatctttt

SEQ ID NO: 29 - DNA, 34 bp, Homo sapiens
ctattattgtgtaacaaattacccccaaacttag

SEQ ID NO: 30 - protein, 343 aa, Artificial Sequence
  Recombinant peptide
MSDLQTLHQNLSALLVDATSDEARKNLMDVFRDRQAFSEATWNTLLSVCRSWAAWCKSNNREWFPAEPEDVRDYLLHLQVRGLAVNTIQRHLALLNMLHRRSGLPRPGDSNAVSLVMRRIRKENVDAGERVRQALAFERTDFDQVRSLMGSSDRRNDIRNLAFLGVAYNTLLRISEIARIRIKDISRTDGGRMLVHIGRTKTLVSTAGVEKALSLGVTRLVERWISVSGVAGDPNNYLFCRVRKNGVAAPSASSQLTTDVLRGIFVAAHRLVHGARDDSGQRYLTWSGHSARVGAARDMARAGVSIAEIMQAGGWTTVESVMSYLRNLDSETGAMVRLLEDGD

SEQ ID NO: 31 - protein, 343 aa, Artificial Sequence
  Recombinant peptide
MSNLQALHQNPSTLLADATCDEVRKNLMDMFRDSQAFSEHTWKALQSVCRSWVAWCKLNSRRWFPAEPEDVRDYLLHLQTRGLAVKTIQHHLGSLNMLHRQSGLPRPGDSNAVSLVMRRIRRENVDAGERAKQALAFERADLDQVRALMENSDRCQDIRNLAFLGVAYNTLLRIGEIARIRVRDISRTDGGRMLIHIGRTKTLVSTAGVEKALSLGVTKLVERWISVSGVADDPDNYLFCRVRRYGVAKPSATSQLSTYALQGIFGAAHRLVYGAKDDSGQKYLAWSGHSARVGAARDMARAGVSLPEIMQAGGWTTVNSVMNYIRNLDSETGAMVRLLEDDD

SEQ ID NO: 32 - protein, 343 aa, Artificial Sequence
  Recombinant protein
MSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDSGQGYLTWSGHSARVGAARDMARAGVSIAEIMQAGGWTTVESVMSYLRNLDSETGAMVRLLEDGD

SEQ ID NO: 33 - protein, 343 aa, Artificial Sequence
  Recombinant protein
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDSGQKYLAWSGHSARVGAARDMARAGVPIPEIMQAGGWTTVNSVMNYIRNLDSETGAMVRLLEDSD

SEQ ID NO: 34 - DNA, 1032 bp, Artificial Sequence
  Recombinant DNA
atgtccgatttacagaccttacaccaaaatttgtctgcattactggtcgatgcaacgagcgatgaggctcgcaagaacctgatggacgtgttcagggaccgccaggcgttttctgaggcaacctggaatacgctcctgtccgtttgccggtcgtgggcagcatggtgcaagtcgaataaccgggaatggtttcccgcagaacctgaagatgttcgcgattatcttctacacctccaggtacgcggtctggcagtaaacactatccagcgacatttggcgctgctgaacatgcttcaccgtcggtccgggctgccacgaccaggcgacagcaatgctgtttcactggttatgcggcgtatccggaaagaaaacgttgatgccggtgagcgtgtaagacaggctctggcgttcgaacgcaccgatttcgaccaggttcgttcactcatggggagtagcgatcggcgcaatgatatacgtaatctggcatttcttggggttgcatataacaccctgttacgtatatccgaaatcgccaggattcggataaaagatatctcgcgtactgacggtgggaggatgctagtccatattggcagaacgaaaacgctggtcagcaccgcaggcgtagagaaagcacttagcctgggggtaaccagactggtcgagcgatggatttccgtctctggcgtagctggtgatccgaataactacctgttttgccgggtcagaaaaaacggtgttgccgcgccatctgcctccagccagctgacaaccgacgtcctgcgagggatttttgtagccgcccaccgcctggtccacggtgccagggatgactctgggcagagatacctgacctggtctggacacagtgcccgcgtcggagccgcgcgggacatggcccgcgctggagtctcaatagcggagattatgcaggctggtggctggaccaccgtagagagcgtcatgagctatctccgtaacctggatagtgaaacgggggcaatggtgcgcctgctggaagacggcgattag

SEQ ID NO: 35 - DNA, 1032 bp, Artificial Sequence
  Recombinant DNA
atgtccaatttacaggccttacaccaaaatccgtccacattactggctgacgcgacgtgtgacgaggttcgcaagaacctgatggacatgttcagggatagccaggcgttttcagagcatacctggaaagcgcttcaatccgtttgccggtcgtgggtggcatggtgcaaactgaacagccggaggtggtttcccgcagagcccgaagatgttcgcgattatcttctgcatcttcagacgcgcggtctggcagtaaaaaccatccagcaccatctgggctcactaaacatgcttcatcgtcagtccgggctgccacgaccaggtgacagcaatgccgtttcactggttatgcggcgaatccgaagagaaaacgttgatgccggtgaacgtgcaaaacaggctctggcgttcgagcgcgctgacctcgaccaggtccgtgcactcatggaaaacagcgatcgctgccaggatatacgtaatctggcatttctgggcgttgcttacaacaccctgttacgaataggcgaaattgccagaatccgggttagagatatctcacgtactgacggtgggagaatgttaatccacattggcagaacgaaaacactggtcagcaccgcaggtgtggagaaggcacttagcctgggggtaactaaactggtcgagcgatggatttccgtctccggtgtagctgatgacccagataactacctgttttgccgggtcagaagatatggcgttgccaagccatctgccaccagccagctgtcaacctacgccctgcaagggatttttggagccgctcaccgcctggtctacggtgctaaggatgactctggtcagaaatacctggcctggtctggacacagtgcccgtgtcggagccgcgcgggatatggcccgcgctggagtttcgttaccggagattatgcaggctggtggctggaccaccgtaaatagtgtcatgaactatatccgtaacctggatagtgagacaggggccatggtgcgcctgctggaagatgacgactag

SEQ ID NO: 36 - DNA, 1032 bp, Artificial Sequence
  Recombinant DNA
atgtccaatctacagaccctacaccagaatttgtccgcactactggccaatgcaacaagtgatgaggctcgcaagaacctgatggacgtcttcagggaccgccgggcgttttctgaagctacctggaaaacgcttctgtccgtttgccggacgtgggcggcatggtgcaagctgaataaccggaaatggtttcccgcagaacctgaagatgtacgcgattatcttctacatctccaggtacgcggtctggcggtgaacacaatccagcgacacttggccctgctaaacatgcttcaccgtcggtccgggctgccgcgaccaggtgacagcagcgctgtttcactggttatgcggcgtatccgaaaggaaaacgttgatgccggtgaacgcgtaaggcaggccctggcgttcgagcgcactgatttcgacaaggttcgttcactcatgggaaacagcgatcgctgccaggatatacgtaatctggcatttctgggtgttgcatataacaccctgctacgcatatccgaaattgccaggattcggataaaagatatctcacgtaccgacggtgggaggatgctaatccatatcggcagaacgaaaacgctggttagcaccgcaggcgtagagaaggcacttagccttggggtaaccagactggtcgggcggtggatttctgtctctggtgtagctggcgacccgaataactacctgttctgccgggtcagaaaaaatggtgttgccgcgccatctgccaccagccagctatcgaccgacgtcctgagaggggtttttgcagccgcccaccgtctggtctacggtaccaaggatgactctggtcaggggtacctgacctggtctggacacagtgcccgcgtcggagccgcgcgggatatggcccgcgctggggtttcaatagcggagattatgcaggctggtggctggaccaccgtagagagcgttatgagctatctccgtaacctggatagtgagacaggtgcaatggtgcgcctgctggaggatggcgattag

SEQ ID NO: 37 - DNA, 1032 bp, Artificial Sequence
  Recombinant DNA
atgtccaatatacagaccccacaccaaagtttgtctgcattactgactgatgcaacgagtgatgtgactcgcaagaacctggcggacatgttcagggatagccaggcgttttcagagcatacctggaaaatgctcctgtccgtttgccggtcgtgggcggcatggtgcgagctgaataaccggaaatggcttcccgtagaacctgaggatgttcgcgattacctcctgcaccttcagacgcgtggtctggcggtaaaaactatccagcatcatctgggctcgctaaacatgcttcaccgtcgggctgggctgccacgaccgggtgacagcaatgctgtttcactggttatgcggcgtatccgaagagaaaacgttgatgccggtgaacgcgcacaacaggccctggcgttcgaacgcaccgatttcgaccaggttcggtcactcgtggaaaacagcgaccgctgccaggatatacgtaatctggcatttctgggggtcgcttataacaccctgttgcgtatatccgaaattgccaggattcgggttaaggatatctcacgtactgacggtgggaggatgttaatccatattggcagaacgaaaacgctggttagtaccgcaggtgtagagaaggcactcagcctgggggtgaccaaactggtcgagcgatggatttccgtctccggtgtagccgatgacccaaataactacctgttttgccgggtcagaagatatggcgttgccaagccgtctgccaccagccagctgtcaacttacgccctgcaagggattttcggagccgcccaccgcctggtctacggtgccaagggtgactctggccagaagtacctggcctggtcgggacacagtgcccgtgtcggagccgcacgagacatggcccgcgctggagttccaataccggagattatgcaggctggtggctggaccaccgtgaatagtgtcatgaactatatccgtaacctggatagcgagacaggagcaatggtgcgcctgctggaagacagcgattag

SEQ ID NO: 38 - DNA, 25 bp, Artificial Sequence
  Artificial oligonucleotide
tcgagggtggcagcggcggtagcat

SEQ ID NO: 39 - DNA, 25 bp, Artificial Sequence
  Synthetic oligonucleotide
gtacatgctaccgccgctgccaccc

SEQ ID NO: 40 - DNA, 43 bp, Artificial Sequence
  Synthetic oligonucleotide
tcgagggcggtagcggtggctctggcggcagcggtggtagcat

SEQ ID NO: 41 - DNA, 43 bp, Artificial Sequence
  Synthetic oligonucleotide
gtacatgctaccaccgctgccgccagagccaccgctaccgccc

SEQ ID NO: 42 - DNA, 61 bp, Artificial Sequence
  Syntheti oligonucleotide
tcgagggcggtagcggtggctctggcggcagcggtggtagcggtggctctggcggtagtat

SEQ ID NO: 43 - DNA, 60 bp, Artificial Sequence
  Synthetic oligonucleotide
tacatactaccgccagagccaccgctaccaccgctgccgccagagccaccgctaccgccc

SEQ ID NO: 44 - DNA, 75 bp, Artificial Sequence
  Synthetic oligonucleotide
tcgagggcggtagcggtggctctggcggcagcggtggtagcggtggctctggcggtagtggtggtagtggcggca

SEQ ID NO: 45 - DNA, 66 bp, Artificial Sequence
  Synthetic oligonucleotide
gtacatgctgccgccactaccaccactaccgccagagccaccgctaccaccgctgccgccagagcc

SEQ ID NO: 46 - DNA, 97 bp, Artificial Sequence
  Synthetic oligonucleotide
tcgagggcggtagcggtggctctggcggcagcggtggtagcggtggctctggcggtagtggtggtagtggcggcagcggtggctctggcggtagtat

SEQ ID NO: 47 - DNA, 97 bp, Artificial Sequence
  Synthetic oligonucleotide
gtacatactaccgccagagccaccgctgccgccactaccaccactaccgccagagccaccgctaccaccgctgccgccagagccaccgctaccgccc

SEQ ID NO: 48 - DNA, 115 bp, Artificial Sequence
  Synthetic oligonucleotide
tcgagggcggtagcggtggctctggcggcagcggtggtagcggtggctctggcggtagtggtggtagtggcggcagcggtggctctggcggtagtggtggtagcggtggctctat

SEQ ID NO: 49 - DNA, 115 bp, Artificial Sequence
  Synthetic oligonucleotide
gtacatagagccaccgctaccaccactaccgccagagccaccgctgccgccactaccaccactaccgccagagccaccgctaccaccgctgccgccagagccaccgctaccgccc

SEQ ID NO: 50 - DNA, 133 bp, Artificial Sequence
  Synthetic oligonucleotide
tcgagggcggtagcggtggctctggcggcagcggtggtagcggtggctctggcggtagtggtggtagtggcggcagcggtggctctggcggtagtggtggtagcggtggctctggaggtagtggcggatcaat

SEQ ID NO: 51 - DNA, 133 bp, Artificial Sequence
  Synthetic oligonucleotide
gtacattgatccgccactacctccagagccaccgctaccaccactaccgccagagccaccgctgccgccactaccaccactaccgccagagccaccgctaccaccgctgccgccagagccaccgctaccgccc

SEQ ID NO: 52 - DNA, 128 bp, Artificial Sequence
  Synthetic oligonucleotide
  variation (29)..(46): r is a or g, v is a or c or g, m is a or c
  variation (83)..(100): r is a or g, v is a or c or g, m is a or c
gaagtgccattccgcctgacctctcgagrvmrvmrvmrvmrvmrvmggcggcagcggtggtagcggtggctctggcggtagtrvmrvmrvmrvmrvmrvmatgtacaaggctaggtggaggctcagtg

SEQ ID NO: 53 - DNA, 128 bp, Artificial Sequence
  Synthetic oligonucleotide
  variation (47)..(82): r is a or g, v is a or c or g, m is a or c
gaagtgccattccgcctgacctctcgagggcggtagcggtggctctrvmrvmrvmrvmrvmrvmrvmrvmrvmrvmrvmrvmggtggtagtggcggcagcatgtacaaggctaggtggaggctcagtg

SEQ ID NO: 54 - DNA, 128 bp, Artificial Sequence
  Synthetic oligonucleotide
  variation (29)..(100): r is a or g, v is a or c or g, m is a or c
gaagtgccattccgcctgacctctcgagrvmrvmrvmrvmrvmrvmrvmrvmrvmrvmrvmrvmrvmrvmrvmrvmrvmrvmrvmrvmrvmrvmrvmrvmatgtacaaggctaggtggaggctcagtg

SEQ ID NO: 55 - DNA, 72 bp, Artificial Sequence
  Recombinant oligonucleotide
gccgaagcaacaagcgaaggcggcagcggtggtagcggtggctctggcggtagtaacggcgcaagaagaacc

SEQ ID NO: 56 - DNA, 72 bp, Artificial Sequence
  Recombinant oligonucleotide
gcaggaacaaccgcaagaggcggcagcggtggtagcggtggctctggcggtagtggaagaagaggagcaaaa

SEQ ID NO: 57 - DNA, 72 bp, Artificial Sequence
  Recombinant oligonucleotide
aaaaacggaagaggaagaggcggcagcggtggtagcggtggctctggcggtagtagaacaaaaagagaaaca

SEQ ID NO: 58 - DNA, 72 bp, Artificial Sequence
  recombinant oligonucleotide
ggcggtagcggtggctctaccgccgccaaagaaggagcagcaagcagcgccagcggtggtagtggcggcagc

SEQ ID NO: 59 - DNA, 72 bp, Artificial Sequence
  Recombinant oligonucleotide
ggcggtagcggtggctctaacagcagaagcaacacagaaaacagcgacaaaggcggtggtagtggcggcagc

SEQ ID NO: 60 - DNA, 72 bp, Artificial Sequence
  Recombinant oligonucleotide
ggcggtagcggtggctctaacggcgaagaaggaaccgaaagaggcaaagcaacaggtggtagtggcggcagc

SEQ ID NO: 61 - DNA, 72 bp, Artificial Sequence
  Synthetic oligonucleotide
ggcggtagcggtggctctaccaccaaagcaaacagagcaaaaggcggcagaggaggtggtagtggcggcagc

SEQ ID NO: 62 - DNA, 72 bp, Artificial Sequence
  Recombinant oligonucleotide
ggcgcaaacgaagacacaaacacagaagcagccggaagcgaaggcaacgaaaaaaccggaacaaacagcgct

SEQ ID NO: 63 - DNA, 72 bp, Artificial Sequence
  Recombinant oligonucleotide
ggcgaaagcagagcagaagacggcgcaaaaggaaacggcagaggcaaaggagaagcaacagcaggcgcagca

SEQ ID NO: 64 - DNA, 2148 bp, Artificial Sequence
  Recombinant DNA
atgtccaatttacaggccttacaccaaaatccgtccacattactggctgacgcgacgtgtgacgaggttcgcaagaacctgatggacatgttcagggatagccaggcgttttcagagcatacctggaaagcgcttcaatccgtttgccggtcgtgggtggcatggtgcaaactgaacagccggaggtggtttcccgcagagcccgaagatgttcgcgattatcttctgcatcttcagacgcgcggtctggcagtaaaaaccatccagcaccatctgggctcactaaacatgcttcatcgtcagtccgggctgccacgaccaggtgacagcaatgccgtttcactggttatgcggcgaatccgaagagaaaacgttgatgccggtgaacgtgcaaaacaggctctggcgttcgagcgcgctgacctcgaccaggtccgtgcactcatggaaaacagcgatcgctgccaggatatacgtaatctggcatttctgggcgttgcttacaacaccctgttacgaataggcgaaattgccagaatccgggttagagatatctcacgtactgacggtgggagaatgttaatccacattggcagaacgaaaacactggtcagcaccgcaggtgtggagaaggcacttagcctgggggtaactaaactggtcgagcgatggatttccgtctccggtgtagctgatgacccagataactacctgttttgccgggtcagaagatatggcgttgccaagccatctgccaccagccagctgtcaacctacgccctgcaagggatttttggagccgctcaccgcctggtctacggtgctaaggatgactctggtcagaaatacctggcctggtctggacacagtgcccgtgtcggagccgcgcgggatatggcccgcgctggagtttcgttaccggagattatgcaggctggtggctggaccaccgtaaatagtgtcatgaactatatccgtaacctggatagtgagacaggggccatggtgcgcctgctggaagatgacgactcgctcgagggcggtagcggtggctctaccaccaaagcaaacagagcaaaaggcggcagaggaggtggtagtggcggcagcatgtacatgtccgatttacagaccttacaccaaaatttgtctgcattactggtcgatgcaacgagcgatgaggctcgcaagaacctgatggacgtgttcagggaccgccaggcgttttctgaggcaacctggaatacgctcctgtccgtttgccggtcgtgggcagcatggtgcaagtcgaataaccgggaatggtttcccgcagaacctgaagatgttcgcgattatcttctacacctccaggtacgcggtctggcagtaaacactatccagcgacatttggcgctgctgaacatgcttcaccgtcggtccgggctgccacgaccaggcgacagcaatgctgtttcactggttatgcggcgtatccggaaagaaaacgttgatgccggtgagcgtgtaagacaggctctggcgttcgaacgcaccgatttcgaccaggttcgttcactcatggggagtagcgatcggcgcaatgatatacgtaatctggcatttcttggggttgcatataacaccctgttacgtatatccgaaatcgccaggattcggataaaagatatctcgcgtactgacggtgggaggatgctagtccatattggcagaacgaaaacgctggtcagcaccgcaggcgtagagaaagcacttagcctgggggtaaccagactggtcgagcgatggatttccgtctctggcgtagctggtgatccgaataactacctgttttgccgggtcagaaaaaacggtgttgccgcgccatctgcctccagccagctgacaaccgacgtcctgcgagggatttttgtagccgcccaccgcctggtccacggtgccagggatgactctgggcagagatacctgacctggtctggacacagtgcccgcgtcggagccgcgcgggacatggcccgcgctggagtctcaatagcggagattatgcaggctggtggctggaccaccgtagagagcgtcatgagctatctccgtaacctggatagtgaaacgggggcaatggtgcgcctgctggaagacggcgattag

SEQ ID NO: 65 - DNA, 2148 bp, Artificial Sequence
  Recombinant DNA
atgtccaatatacagaccccacaccaaagtttgtctgcattactgactgatgcaacgagtgatgtgactcgcaagaacctggcggacatgttcagggatagccaggcgttttcagagcatacctggaaaatgctcctgtccgtttgccggtcgtgggcggcatggtgcgagctgaataaccggaaatggcttcccgtagaacctgaggatgttcgcgattacctcctgcaccttcagacgcgtggtctggcggtaaaaactatccagcatcatctgggctcgctaaacatgcttcaccgtcgggctgggctgccacgaccgggtgacagcaatgctgtttcactggttatgcggcgtatccgaagagaaaacgttgatgccggtgaacgcgcacaacaggccctggcgttcgaacgcaccgatttcgaccaggttcggtcactcgtggaaaacagcgaccgctgccaggatatacgtaatctggcatttctgggggtcgcttataacaccctgttgcgtatatccgaaattgccaggattcgggttaaggatatctcacgtactgacggtgggaggatgttaatccatattggcagaacgaaaacgctggttagtaccgcaggtgtagagaaggcactcagcctgggggtgaccaaactggtcgagcgatggatttccgtctccggtgtagccgatgacccaaataactacctgttttgccgggtcagaagatatggcgttgccaagccgtctgccaccagccagctgtcaacttacgccctgcaagggattttcggagccgcccaccgcctggtctacggtgccaagggtgactctggccagaagtacctggcctggtcgggacacagtgcccgtgtcggagccgcacgagacatggcccgcgctggagttccaataccggagattatgcaggctggtggctggaccaccgtgaatagtgtcatgaactatatccgtaacctggatagcgagacaggagcaatggtgcgcctgctggaagacagcgattcgctcgagggcggtagcggtggctctaccaccaaagcaaacagagcaaaaggcggcagaggaggtggtagtggcggcagcatgtacatgtccaatctacagaccctacaccagaatttgtccgcactactggccaatgcaacaagtgatgaggctcgcaagaacctgatggacgtcttcagggaccgccgggcgttttctgaagctacctggaaaacgcttctgtccgtttgccggacgtgggcggcatggtgcaagctgaataaccggaaatggtttcccgcagaacctgaagatgtacgcgattatcttctacatctccaggtacgcggtctggcggtgaacacaatccagcgacacttggccctgctaaacatgcttcaccgtcggtccgggctgccgcgaccaggtgacagcagcgctgtttcactggttatgcggcgtatccgaaaggaaaacgttgatgccggtgaacgcgtaaggcaggccctggcgttcgagcgcactgatttcgacaaggttcgttcactcatgggaaacagcgatcgctgccaggatatacgtaatctggcatttctgggtgttgcatataacaccctgctacgcatatccgaaattgccaggattcggataaaagatatctcacgtaccgacggtgggaggatgctaatccatatcggcagaacgaaaacgctggttagcaccgcaggcgtagagaaggcacttagccttggggtaaccagactggtcgggcggtggatttctgtctctggtgtagctggcgacccgaataactacctgttctgccgggtcagaaaaaatggtgttgccgcgccatctgccaccagccagctatcgaccgacgtcctgagaggggtttttgcagccgcccaccgtctggtctacggtaccaaggatgactctggtcaggggtacctgacctggtctggacacagtgcccgcgtcggagccgcgcgggatatggcccgcgctggggtttcaatagcggagattatgcaggctggtggctggaccaccgtagagagcgttatgagctatctccgtaacctggatagtgagacaggtgcaatggtgcgcctgctggaggatggcgattag

SEQ ID NO: 66 - protein, 343 aa, Artificial Sequence
  Recombinant protein
MSNLQTLHQNLSALLADATSDETRKNLMDVLRDSQAFSEQTWKSLLFVCRSWAAWCELNNREWFPAEPEDVRDYLLHLQARGLAVKTIQNHLGSLNMLHRRSGLPRPGDSNAVSLVMRRIRKENVDAGERAKQALAFERADFSRVRALMENSDRPQDIRNLAFLGVAYNTLLRISEIARIRVKDISRTDGGRMLIHIGRTKTLVSTAGVEKALSLGVTKLVERWISVSGVVDDPNNYLFCRVRRGGVAKPSATSQLSTCALQGIFAAAHRLLYGAKDDSGQRYLAWSGHSARVGAARDMARAGVPIVQIMQAGGWTTVESVMNYIRNLDSETGAMVRLLEDGD

SEQ ID NO: 67 - DNA, 1032 bp, Artificial Sequence
  Recombinant DNA
atgtcaaatttacagaccttacaccagaacttgtccgcactactggccgatgcaacgagcgatgagactcgtaagaacctgatggatgtgctcagggatagccaggcgttttccgagcaaacctggaaatcactcctgttcgtttgccggtcgtgggcggcatggtgcgagctgaataaccgggaatggttccctgcggaacctgaagatgtccgcgattatcttctgcaccttcaggcgcgcggtctggcagtaaaaactatccagaaccatttgggctcgctaaacatgctccaccgtcggtccgggctgccacggccaggcgacagcaatgccgtttcactggttatgcgacgaatccgaaaagaaaacgttgatgccggtgaacgtgcaaagcaggccctggcgttcgaacgcgccgacttcagccgagttcgtgcgctcatggaaaatagcgatcgcccccaggatatacgtaatctggcctttctgggggttgcttataacaccctgttacgcatatccgagattgccaggattcgggttaaggacatctcacgtactgacggtgggagaatgttaatccacatcggcaggacgaaaacgctggtcagcaccgcaggcgtagagaaggcgcttagcctgggggtaaccaagctggtcgagcggtggatctccgtctctggtgtagtcgatgacccgaataactacctgttctgcagggtcagacgaggtggtgttgccaagccatctgccaccagccagctatcaacttgcgccctgcaagggatttttgcagccgcccaccgtctgctctacggtgccaaggatgactctggtcagagatacctggcctggtctggacacagcgcccgcgtcggggccgcgcgggacatggcccgcgccggggttccaatagtgcagattatgcaggctggtggctggaccaccgtagagagtgtcatgaactatatccgtaacctggatagtgaaacaggagcaatggtacgtctgctggaggacggcgattag

SEQ ID NO: 68 - protein, 343 aa, Homo sapiens
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 69 - protein, 343 aa, Artificial Sequence
  Recombinant protein
MSNLLTLHHSLPALPADATSDEVRKNLMDVFRDRPAFSEHTWEMLLSVCRSWAAWCKLNNRKWFPAEPEDVRDYLLHLQARGLAVKTIQQHLCRLNMLHRRSGLPRPSDSNAVSLVMRRIRKENVDAGERTKQALAFERTDFDQVRSLMENSDRCQDIRNLAFLGVAYNTLLRIAEIARIRVKDISRTDGGRMLIHIGRTKTLVSTAGVEKALSLGVTKLVERWISVSGVADDPNNYLFCRVRRYGVAAPSATSQLSTYALQRIFEATHRLIYGAKDDSGQRYLAWSGHSARVGAARDMARAGVSIPEIMQAGGWTTVNSVMNYIRNLDSETGAMVRLLEDGD

SEQ ID NO: 70 - protein, 343 aa, Artificial Sequence
  Recombinant protein
MSILLTLHQSLSALLVDATSDEARKNLMDVLRDRQAFSERTWKVLLSVCRTWAAWCKLNNRKWFPAEPEDVRDYLLHLQARGLAVNTILQHLAQLNMLHRRFGLPRPGDSDAVSLVMRRIRRENVDAGERTKQALAFERTDFDQVRALMENSERGQDIRTLALLGVAYNTLLRVSEIARIRIKDISRTDGGRMLIHISRTKTLVSTAGVEKALSLGVTKLVERWISVSGVASDPNNYLFCQVRINGVAVPSATSRLSTDVLRKIFEAAHRLIYGAKDGSGQRYLAWSGHSARVGAARDMARAGVSIAEIMQAGGWTTVESVMNYIRNLDSETGAMVRLLEDGD

SEQ ID NO: 71 - protein, 715 aa, Artificial Sequence
  Rcombinant protein
MSNLQALHQNPSTLLADATCDEVRKNLMDMFRDSQAFSEHTWKALQSVCRSWVAWCKLNSRRWFPAEPEDVRDYLLHLQTRGLAVKTIQHHLGSLNMLHRQSGLPRPGDSNAVSLVMRRIRRENVDAGERAKQALAFERADLDQVRALMENSDRCQDIRNLAFLGVAYNTLLRIGEIARIRVRDISRTDGGRMLIHIGRTKTLVSTAGVEKALSLGVTKLVERWISVSGVADDPDNYLFCRVRRYGVAKPSATSQLSTYALQGIFGAAHRLVYGAKDDSGQKYLAWSGHSARVGAARDMARAGVSLPEIMQAGGWTTVNSVMNYIRNLDSETGAMVRLLEDDDSLEGGSGGSTTKANRAKGGRGGGSGGSMYMSDLQTLHQNLSALLVDATSDEARKNLMDVFRDRQAFSEATWNTLLSVCRSWAAWCKSNNREWFPAEPEDVRDYLLHLQVRGLAVNTIQRHLALLNMLHRRSGLPRPGDSNAVSLVMRRIRKENVDAGERVRQALAFERTDFDQVRSLMGSSDRRNDIRNLAFLGVAYNTLLRISEIARIRIKDISRTDGGRMLVHIGRTKTLVSTAGVEKALSLGVTRLVERWISVSGVAGDPNNYLFCRVRKNGVAAPSASSQLTTDVLRGIFVAAHRLVHGARDDSGQRYLTWSGHSARVGAARDMARAGVSIAEIMQAGGWTTVESVMSYLRNLDSETGAMVRLLEDGD

SEQ ID NO: 72 - protein, 715 aa, Artificial Sequence
  Recombinant protein
MSNIQTPHQSLSALLTDATSDVTRKNLADMFRDSQAFSEHTWKMLLSVCRSWAAWCELNNRKWLPVEPEDVRDYLLHLQTRGLAVKTIQHHLGSLNMLHRRAGLPRPGDSNAVSLVMRRIRRENVDAGERAQQALAFERTDFDQVRSLVENSDRCQDIRNLAFLGVAYNTLLRISEIARIRVKDISRTDGGRMLIHIGRTKTLVSTAGVEKALSLGVTKLVERWISVSGVADDPNNYLFCRVRRYGVAKPSATSQLSTYALQGIFGAAHRLVYGAKGDSGQKYLAWSGHSARVGAARDMARAGVPIPEIMQAGGWTTVNSVMNYIRNLDSETGAMVRLLEDSDSLEGGSGGSTTKANRAKGGRGGGSGGSMYMSNLQTLHQNLSALLANATSDEARKNLMDVFRDRRAFSEATWKTLLSVCRTWAAWCKLNNRKWFPAEPEDVRDYLLHLQVRGLAVNTIQRHLALLNMLHRRSGLPRPGDSSAVSLVMRRIRKENVDAGERVRQALAFERTDFDKVRSLMGNSDRCQDIRNLAFLGVAYNTLLRISEIARIRIKDISRTDGGRMLIHIGRTKTLVSTAGVEKALSLGVTRLVGRWISVSGVAGDPNNYLFCRVRKNGVAAPSATSQLSTDVLRGVFAAAHRLVYGTKDDSGQGYLTWSGHSARVGAARDMARAGVSIAEIMQAGGWTTVESVMSYLRNLDSETGAMVRLLEDGD

SEQ ID NO: 73 - DNA, 34 bp, Homo sapiens
ataacttcgtataatgtatgctatacgaagttat

SEQ ID NO: 74 - DNA, 32 bp, Homo sapiens
taactttaaataatgccaattatttaaagtta

SEQ ID NO: 75 - DNA, 34 bp, Homo sapiens
tcaatttctgagaatgacagttctcggaaattga

SEQ ID NO: 76 - DNA, 34 bp, Homo sapiens
ctcgtgtccgataactgtaattatcggacatgat

SEQ ID NO: 77 - DNA, 34 bp, Homo sapiens
aataggtctgagaatgggcgttctcagacgtatt

SEQ ID NO: 78 - DNA, 21 bp, Homo sapiens
cagcttttttatactaagttg

SEQ ID NO: 79 - DNA, 34 bp, Homo sapiens
gaagttcctatactttctagagaataggaacttc

SEQ ID NO: 80 - DNA, 31 bp, Homo sapiens
ttgatgaaagaataacgtattctttcatcaa

SEQ ID NO: 81 - DNA, 43 bp, Homo sapiens
acgaaaaatggtaaggaatagaccattccttaccatttttcgt

SEQ ID NO: 82 - DNA, 61 bp, Homo sapiens
aaacgatatcagacatttgtctgataatgcttcattatcagacaaatgtctgatatcgttt

SEQ ID NO: 83 - DNA, 39 bp, Homo sapiens
gagtttcattaaggaataactaattccctaatgaaactc

SEQ ID NO: 84 - DNA, 33 bp, Homo sapiens
ggttgcttaagaataagtaattcttaagcaacc

SEQ ID NO: 85 - DNA, 34 bp, Homo sapiens
tgaatgtcctataattacacttataggacattca

SEQ ID NO: 86 - DNA, 34 bp, Homo sapiens
gaaactttaaataataagtcttatttaaagtttc

SEQ ID NO: 87 - DNA, 34 bp, Homo sapiens
ataaatatgtgtgtatatatacacacaaacatat

SEQ ID NO: 88 - DNA, 34 bp, Homo sapiens
atatatctgtgtatatatatacacacacacatat

SEQ ID NO: 89 - DNA, 34 bp, Homo sapiens
atatatatgtgtatatatatacacacatacatat

SEQ ID NO: 90 - DNA, 34 bp, Homo sapiens
atatatgtgtgtatatatatacacacacacatac

SEQ ID NO: 91 - DNA, 34 bp, Homo sapiens
ataaatatgtgtaaactaaacaacacactcttaa

SEQ ID NO: 92 - DNA, 34 bp, Homo sapiens
acaaatatgtggaaactaaacaacacattcttga

SEQ ID NO: 93 - protein, 343 aa, Artificial sequence
  Recombinant protein
MSSLQTLHQNLSALLVDATSDEARENLIGGFRDRQAFSEATWKTLLSVCRSWAAWCKLNSRKWFPAEPEDVRDYLLHLQVRGLAVNTIQRHLALLNMLHRRSGLPRPGDSNAVSLVMRRIRKENVDAGERVKQALAFERTDFDRVRSLMENSDRCQDIRNLAFLGVAYNTLLRVSEIARIRVKDISRTDGGRMLIHIGRTKTLVSTAGVEEALSLGVTRLVERWISVSGVAGDPNNYLFCRVRKNGVAAPSATSQLTTDVLRGIFVAAHRLIYGARDDSGQRYLAWSGHSARVGAARDMARAGVSIAEIMQAGGWTTVESVMSYLRNLDSETGAMVRLLEDGD

SEQ ID NO: 94 - protein, 343 aa, Artificial sequence
  Recombinant protein
MSSLPTLHQSLSALLVDAASGEVRTNLMVMFRDSQAFSVHTWKMLLSVCRSWAAWCELNNRKWFPADPEDVRDYLLHLQARGLAVKTIQHHLGSLDMLHRRAGLPRPGDSNAVSLVMRRIRRANVDAGERAGQALAFERTDFDQVRSLMENSDRCQDIRNLAFLGVAYNTLLRISEIARIRVRDISRTDGGRMLIHIGRTKTLVSTAGVEKALSLGVTKLVERWISVSGVADDPNNYLFCRVRRYGVAKPSATSQLSTYALQGIFGAAHRLVYGVKDDSGQKYLAWSGHSARVGAARDMARAGASLPEIMQAGGWTTVNSVMNYIRNLDSETGAMVRLLEDSD

SEQ ID NO: 95 - DNA, 1032 bp, Artificial sequence
  Recombinant polynucleotide
atgtccagtttacagaccttacaccagaatctgtcagcattactggtcgatgcaacgagcgatgaggctcgcgagaacctgataggtgggttcagggaccgccaggcgttttctgaggctacctggaagacgcttctgtccgtttgccggtcgtgggcagcatggtgcaagttgaatagccggaaatggtttcccgcagagcctgaagatgttcgcgattatcttctacatctccaggttcgcggtctggctgtaaacactatccagcgacatctggccctgctaaacatgctacaccgtcggtccgggctgccacgaccaggtgacagcaacgctgtttcactggttatgcggcgtatccgaaaagaaaacgttgatgccggtgagcgtgtaaaacaggctctggcgttcgaacgcactgatttcgaccgggttcgttcactcatggaaaatagcgaccgctgccaggatatacgtaatctggcattcctgggggttgcttataacaccctgttacgtgtatccgaaatcgccaggattcgggtaaaagatatctctcgtactgacggtgggagaatgttaatccatattggcagaacgaaaacgctggttagcaccgcaggcgtggaggaggcacttagcctgggggtaaccagactggtcgagcggtggatttccgtctctggtgtggctggtgacccgaataactacctgttttgccgggtcagaaagaacggtgtcgccgcgccatctgccaccagccagctaacaaccgatgtcctgcgagggatttttgtagccgcccaccgtctgatctacggtgccagggatgactctggtcagagatacctggcctggtctggacacagtgcccgcgtcggagccgcgcgggatatggcccgcgctggagtctcaatagcggagattatgcaggctggtggctggaccaccgtagagagtgtcatgagctacctccgtaacctggatagtgagacgggggcaatggtgcgcctgctggaggatggtgactag

SEQ ID NO: 96 - DNA, 1032 bp, Artificial sequence
  Recombinant polynucleotide
atgtccagtttaccaaccctacaccaaagtttgtctgcattactggtcgacgcagcgagcggcgaggttcgcacgaacctgatggtcatgttcagggatagccaggcgttttctgtgcatacctggaaaatgcttctgtccgtttgccggtcgtgggcggcatggtgcgagctgaacaaccggaaatggtttcccgcagaccctgaagatgttcgcgattatctcctacaccttcaggcgcgcggtctggcagtaaaaactatccagcatcatttaggctcgctggacatgcttcatcgccgggccgggctgccacgaccaggtgacagcaacgctgtctcactggttatgcggcgcatccgaagagcaaacgttgatgccggcgaacgtgcagggcaggccctggcgttcgaacgcactgacttcgaccaggttcgttcactcatggaaaatagcgatcgctgccaggatatacgtaacctggcattcctgggggtcgcttacaacaccctgttacgtatatccgaaattgccaggatccgggttagagatatctcacgtactgacggtgggaggatgttaatccacatcggcagaacgaaaacgctggtcagcaccgcaggtgtagagaaggcacttagcctgggagtaactaaactggtcgagcggtggatctccgtctctggtgtagctgatgatccaaataactacctgttttgccgggtcagaagatatggtgttgccaagccatctgccaccagccagctatcaacttacgccctgcaagggatttttggagccgcccaccgcctggtctacggtgtcaaggatgactctggtcagaagtacctggcctggtccggacacagtgcccgcgtcggagccgcgcgagatatggcccgcgctggagcttcactaccggagattatgcaggctggtggctggaccactgtaaacagtgtcatgaactatatccgtaacctggatagcgaaacaggagcaatggtacgcttgctggaagatagcgattag

SEQ ID NO: 97 - protein, 715 aa, Artificial seqeunce
  Recombinant protein
MSSLPTLHQSLSALLVDAASGEVRTNLMVMFRDSQAFSVHTWKMLLSVCRSWAAWCELNNRKWFPADPEDVRDYLLHLQARGLAVKTIQHHLGSLDMLHRRAGLPRPGDSNAVSLVMRRIRRANVDAGERAGQALAFERTDFDQVRSLMENSDRCQDIRNLAFLGVAYNTLLRISEIARIRVRDISRTDGGRMLIHIGRTKTLVSTAGVEKALSLGVTKLVERWISVSGVADDPNNYLFCRVRRYGVAKPSATSQLSTYALQGIFGAAHRLVYGVKDDSGQKYLAWSGHSARVGAARDMARAGASLPEIMQAGGWTTVNSVMNYIRNLDSETGAMVRLLEDSDSLEGGSGGSTTKANRAKGGRGGGSGGSMYMSSLQTLHQNLSALLVDATSDEARENLIGGFRDRQAFSEATWKTLLSVCRSWAAWCKLNSRKWFPAEPEDVRDYLLHLQVRGLAVNTIQRHLALLNMLHRRSGLPRPGDSNAVSLVMRRIRKENVDAGERVKQALAFERTDFDRVRSLMENSDRCQDIRNLAFLGVAYNTLLRVSEIARIRVKDISRTDGGRMLIHIGRTKTLVSTAGVEEALSLGVTRLVERWISVSGVAGDPNNYLFCRVRKNGVAAPSATSQLTTDVLRGIFVAAHRLIYGARDDSGQRYLAWSGHSARVGAARDMARAGVSIAEIMQAGGWTTVESVMSYLRNLDSETGAMVRLLEDGD

SEQ ID NO: 98 - DNA, 2148 bp, Artificial sequence
  Recombinant polynucleotide
atgtccagtttaccaaccctacaccaaagtttgtctgcattactggtcgacgcagcgagcggcgaggttcgcacgaacctgatggtcatgttcagggatagccaggcgttttctgtgcatacctggaaaatgcttctgtccgtttgccggtcgtgggcggcatggtgcgagctgaacaaccggaaatggtttcccgcagaccctgaagatgttcgcgattatctcctacaccttcaggcgcgcggtctggcagtaaaaactatccagcatcatttaggctcgctggacatgcttcatcgccgggccgggctgccacgaccaggtgacagcaacgctgtctcactggttatgcggcgcatccgaagagcaaacgttgatgccggcgaacgtgcagggcaggccctggcgttcgaacgcactgacttcgaccaggttcgttcactcatggaaaatagcgatcgctgccaggatatacgtaacctggcattcctgggggtcgcttacaacaccctgttacgtatatccgaaattgccaggatccgggttagagatatctcacgtactgacggtgggaggatgttaatccacatcggcagaacgaaaacgctggtcagcaccgcaggtgtagagaaggcacttagcctgggagtaactaaactggtcgagcggtggatctccgtctctggtgtagctgatgatccaaataactacctgttttgccgggtcagaagatatggtgttgccaagccatctgccaccagccagctatcaacttacgccctgcaagggatttttggagccgcccaccgcctggtctacggtgtcaaggatgactctggtcagaagtacctggcctggtccggacacagtgcccgcgtcggagccgcgcgagatatggcccgcgctggagcttcactaccggagattatgcaggctggtggctggaccactgtaaacagtgtcatgaactatatccgtaacctggatagcgaaacaggagcaatggtacgcttgctggaagatagcgattcgctcgagggcggtagcggtggctctaccaccaaagcaaacagagcaaaaggcggcagaggaggtggtagtggcggcagcatgtacatgtccagtttacagaccttacaccagaatctgtcagcattactggtcgatgcaacgagcgatgaggctcgcgagaacctgataggtgggttcagggaccgccaggcgttttctgaggctacctggaagacgcttctgtccgtttgccggtcgtgggcagcatggtgcaagttgaatagccggaaatggtttcccgcagagcctgaagatgttcgcgattatcttctacatctccaggttcgcggtctggctgtaaacactatccagcgacatctggccctgctaaacatgctacaccgtcggtccgggctgccacgaccaggtgacagcaacgctgtttcactggttatgcggcgtatccgaaaagaaaacgttgatgccggtgagcgtgtaaaacaggctctggcgttcgaacgcactgatttcgaccgggttcgttcactcatggaaaatagcgaccgctgccaggatatacgtaatctggcattcctgggggttgcttataacaccctgttacgtgtatccgaaatcgccaggattcgggtaaaagatatctctcgtactgacggtgggagaatgttaatccatattggcagaacgaaaacgctggttagcaccgcaggcgtggaggaggcacttagcctgggggtaaccagactggtcgagcggtggatttccgtctctggtgtggctggtgacccgaataactacctgttttgccgggtcagaaagaacggtgtcgccgcgccatctgccaccagccagctaacaaccgatgtcctgcgagggatttttgtagccgcccaccgtctgatctacggtgccagggatgactctggtcagagatacctggcctggtctggacacagtgcccgcgtcggagccgcgcgggatatggcccgcgctggagtctcaatagcggagattatgcaggctggtggctggaccaccgtagagagtgtcatgagctacctccgtaacctggatagtgagacgggggcaatggtgcgcctgctggaggatggtgactag

SEQ ID NO: 99 - protein, 343 aa, Artificial squence
  Recombinant protein
MSNLQTIHQNLTASLADATSDEARKNLMDVLRDRQAFSEATWKTLLSVCRTWAAWCKSNNRKWFPAEPEDVRDYLLHLQVRGLAVNTIQRHLALLNMLHRRSGLPRPGDSNAVSLVMRRIRKENVDAGERVRQALAFERTDLDQVRSLMGDSDRCKDIRNLAFPGVAYNTLLRISEIARIRIKDISRTDGGRMLIHIGRTKTLVSTAGVEKALSLGVTRLVERWISVSGVADDPNNYLFCRVRKNGVAVPSATSQLSTDVLRGIFATAHRLVYGARDGSGQRFLTWSGHSARVGAARDMARAGVSIAEIMQAGGWTTVGSVMNYIRNLDSETGAMVRLLEDGD

SEQ ID NO: 100 - protein, 343 aa, Artificial sequence
  Recombinant protein
MSDLQTLHQKLSALLADATSDEARKNLMDMLRDWQAFSEHTWKMLLSVCRSWAAWCELNSRKWFPAEPEDVRDYLLYLQARGLAVKTIQHHLGSLNMLHRRSGLPRPGDSNAVSLVMRRIRKENVDAGERAKQALAFERTDFDQVRLLMENSDRCQDIRNLAFLGIAYNTLLRISEIARIRVRDISRTDGGRMLIHIGRTKTLVSTAGVEEALSLGVTKLVERWISVSGVADDPDNYLFCRVRRYGVAKPSATSQLSTYALQGIFGAAHRLVYGAKDDSGQKYLAWSGHSARVGAARDMARAGVPIPEIMQAGGWTTVGSVMNYIRNLDSETGAMVRLLEDGD

SEQ ID NO: 101 - DNA, 1032 bp, Artificial Sequence
  Recombinant polynucleotide
atgtccaatttacagaccatacaccagaatctgactgcatcactggccgatgcaacgagcgatgaggctcgcaagaacctgatggacgtgctcagggaccgccaggcgttctctgaggctacctggaagacgctcctgtccgtttgccggacgtgggcagcatggtgcaagtcgaataaccggaaatggtttcccgcagaacctgaagatgttcgcgattatcttctgcatctccaggtacgcggtctggcagtaaacactatccagcgacatctggccctgctaaacatgcttcaccgtcggtccgggctgccacgaccaggtgacagcaatgctgtttcactggttatgcggcgaatccgaaaagaaaacgttgatgccggtgaacgcgtaagacaggccctggcgttcgaacgcactgatctcgaccaggttcgttcactcatgggagatagcgatcggtgcaaggatatacgtaatctggcatttccgggggttgcttataacaccctgttacgtatatccgaaatcgccaggattcggataaaagatatctcacgtactgacggtgggaggatgcttatccatattggcagaacgaaaacgctggttagcaccgcaggcgtggagaaggcactaagcctgggggtgaccagactggtcgagcgatggatttccgtctctggcgtagctgacgatccgaataactacctgttttgccgggtcagaaaaaatggtgttgccgtgccatctgccaccagccagctgtcgaccgacgtcctgcgagggatttttgcgaccgcccaccgtctggtctacggtgccagggatggctctggtcagagattcctgacctggtctggacacagtgcccgcgtcggagccgcgcgggatatggcccgcgctggggtttcaatagcggagattatgcaggctggtggctggaccaccgtaggtagtgtcatgaactatatccgtaacctggacagcgaaacgggagcgatggtgcgcctgctggaagatggcgattag

SEQ ID NO: 102 - DNA, 1032 bp, Artificial Sequence
  Recombinant polynucleotide
atgtccgatctacagaccttacaccaaaaattgtccgcattactggccgacgcaacgagtgatgaggctcgcaagaacctgatggacatgctcagagattggcaggcgttttctgagcatacctggaaaatgctcctgtccgtttgccggtcgtgggcggcatggtgcgagttgaatagccggaaatggtttcccgcggaacctgaagatgttcgcgattatcttctgtatcttcaggcgcgaggcctggcagtaaagactatccagcaccatctgggctcgctgaacatgcttcaccgccggtctgggctgccacgaccaggtgacagcaatgccgtttcactggttatgcggcgaatccgaaaagaaaacgttgatgccggtgaacgtgcgaaacaggctctggcgttcgagcgcactgatttcgaccaggttcgcttactcatggaaaacagcgatcgctgccaggatatacgtaacctggcgtttctggggattgcttataacaccctgttacgtatatccgaaattgccaggatccgggtcagagatatctcacgtactgacggtgggagaatgttgatccatattggcaggacgaaaacgctggttagcaccgcaggtgtagaggaggcgcttagcctgggggtgaccaaactggtcgagcggtggatttctgtctccggtgtagccgatgacccggataactacctgttttgccgggtcagaagatacggtgttgccaagccatctgccaccagccagctatcaacctacgctctgcaagggatttttggggctgcccaccgtctggtctacggtgccaaggatgactctggacagaaatacctggcctggtctggacacagtgcccgtgtcggggccgcgcgggatatggcccgcgctggagttccaataccggagattatgcaggctggtggctggaccaccgtaggtagtgtcatgaactatatccgtaacctggacagcgaaacgggagcgatggtgcgcctgctggaagatggcgattag

SEQ ID NO: 103 - protein, 715 aa, Artificial Sequence
  Recombinant protein
MSDLQTLHQKLSALLADATSDEARKNLMDMLRDWQAFSEHTWKMLLSVCRSWAAWCELNSRKWFPAEPEDVRDYLLYLQARGLAVKTIQHHLGSLNMLHRRSGLPRPGDSNAVSLVMRRIRKENVDAGERAKQALAFERTDFDQVRLLMENSDRCQDIRNLAFLGIAYNTLLRISEIARIRVRDISRTDGGRMLIHIGRTKTLVSTAGVEEALSLGVTKLVERWISVSGVADDPDNYLFCRVRRYGVAKPSATSQLSTYALQGIFGAAHRLVYGAKDDSGQKYLAWSGHSARVGAARDMARAGVPIPEIMQAGGWTTVGSVMNYIRNLDSETGAMVRLLEDGDSLEGGSGGSTTKANRAKGGRGGGSGGSMYMSNLQTIHQNLTASLADATSDEARKNLMDVLRDRQAFSEATWKTLLSVCRTWAAWCKSNNRKWFPAEPEDVRDYLLHLQVRGLAVNTIQRHLALLNMLHRRSGLPRPGDSNAVSLVMRRIRKENVDAGERVRQALAFERTDLDQVRSLMGDSDRCKDIRNLAFPGVAYNTLLRISEIARIRIKDISRTDGGRMLIHIGRTKTLVSTAGVEKALSLGVTRLVERWISVSGVADDPNNYLFCRVRKNGVAVPSATSQLSTDVLRGIFATAHRLVYGARDGSGQRFLTWSGHSARVGAARDMARAGVSIAEIMQAGGWTTVGSVMNYIRNLDSETGAMVRLLEDGD

SEQ ID NO: 104 - DNA, 2148 bp, Artificial Sequence
  Recombinant polynucleotide
atgtccgatctacagaccttacaccaaaaattgtccgcattactggccgacgcaacgagtgatgaggctcgcaagaacctgatggacatgctcagagattggcaggcgttttctgagcatacctggaaaatgctcctgtccgtttgccggtcgtgggcggcatggtgcgagttgaatagccggaaatggtttcccgcggaacctgaagatgttcgcgattatcttctgtatcttcaggcgcgaggcctggcagtaaagactatccagcaccatctgggctcgctgaacatgcttcaccgccggtctgggctgccacgaccaggtgacagcaatgccgtttcactggttatgcggcgaatccgaaaagaaaacgttgatgccggtgaacgtgcgaaacaggctctggcgttcgagcgcactgatttcgaccaggttcgcttactcatggaaaacagcgatcgctgccaggatatacgtaacctggcgtttctggggattgcttataacaccctgttacgtatatccgaaattgccaggatccgggtcagagatatctcacgtactgacggtgggagaatgttgatccatattggcaggacgaaaacgctggttagcaccgcaggtgtagaggaggcgcttagcctgggggtgaccaaactggtcgagcggtggatttctgtctccggtgtagccgatgacccggataactacctgttttgccgggtcagaagatacggtgttgccaagccatctgccaccagccagctatcaacctacgctctgcaagggatttttggggctgcccaccgtctggtctacggtgccaaggatgactctggacagaaatacctggcctggtctggacacagtgcccgtgtcggggccgcgcgggatatggcccgcgctggagttccaataccggagattatgcaggctggtggctggaccaccgtaggtagtgtcatgaactatatccgtaacctggacagcgaaacgggagcgatggtgcgcctgctggaagatggcgattcgctcgagggcggtagcggtggctctaccaccaaagcaaacagagcaaaaggcggcagaggaggtggtagtggcggcagcatgtacatgtccaatttacagaccatacaccagaatctgactgcatcactggccgatgcaacgagcgatgaggctcgcaagaacctgatggacgtgctcagggaccgccaggcgttctctgaggctacctggaagacgctcctgtccgtttgccggacgtgggcagcatggtgcaagtcgaataaccggaaatggtttcccgcagaacctgaagatgttcgcgattatcttctgcatctccaggtacgcggtctggcagtaaacactatccagcgacatctggccctgctaaacatgcttcaccgtcggtccgggctgccacgaccaggtgacagcaatgctgtttcactggttatgcggcgaatccgaaaagaaaacgttgatgccggtgaacgcgtaagacaggccctggcgttcgaacgcactgatctcgaccaggttcgttcactcatgggagatagcgatcggtgcaaggatatacgtaatctggcatttccgggggttgcttataacaccctgttacgtatatccgaaatcgccaggattcggataaaagatatctcacgtactgacggtgggaggatgcttatccatattggcagaacgaaaacgctggttagcaccgcaggcgtggagaaggcactaagcctgggggtgaccagactggtcgagcgatggatttccgtctctggcgtagctgacgatccgaataactacctgttttgccgggtcagaaaaaatggtgttgccgtgccatctgccaccagccagctgtcgaccgacgtcctgcgagggatttttgcgaccgcccaccgtctggtctacggtgccagggatggctctggtcagagattcctgacctggtctggacacagtgcccgcgtcggagccgcgcgggatatggcccgcgctggggtttcaatagcggagattatgcaggctggtggctggaccaccgtaggtagtgtcatgaactatatccgtaacctggacagcgaaacgggagcgatggtgcgcctgctggaagatggcgattag

SEQ ID NO: 105 - protein, 343 aa, Artificial Sequence
  Recombinant protein
MSNLLTLHHSLSALLADAASDEARKNLMDMFRDRQAFSEHTWKMLLFVCRSWAAWCKLNNRKWFPAEPEDVRDYLLHLQARDLAVKTIQQHLGQLNMLHRRSGLPRPGDSNAVSLVMRRIRKENVDAGERVKQALAFERTDFDQVRSLMENSDRCQDIRNLAFLGVAYNTLLRVSEIARIRVKDISCTDGGRMLIHIGRTKTLVSTAGVEKALSLGVTKLVERWISVSGVADDPNNYLFCRVSKYGVASPSATSQLSTHALQRIFEAAHRLIYGAKDDSGQRYLAWSGHSARVGAARDMARAGVSIPEVMQAGGWTTVNSVMNYIRNLDSETGAMVRLLEDGD

SEQ ID NO: 106 - protein, 343 aa, Artificial Sequence
  Recombinant Protein
MSNLLTLHQNLTALLVDATSDEVRKDLMDVLRDSQAFSEHTWRVLLSVCRSWAAWCELNNRKWFPAEPEDVRDYLLHLQARGLTVNTIQQHLAQLNMLHRRSGLPRPGDSNAVSLVMRRIRKENVDAGERVKQALAFERTDFDQVRSLMEDSDRCQDVRNLAFLGVAYSTLLRVSEIARIRVKDISRTDGGRMLIHIGRTKTLVSTAGVERALSLGVTKLVERWISVSGVADDPDNYLFCQVRRSGVAAPSATSQLSTYALQGIFAATHRLIYGAKDGSGQRYLTWSGHSARVGAARDMARAGVSVAEIMQAGGWITVESVMNYIRNLDSETGAMVRLLEDGD